(19) United States
(12) Patent Application Publication
(10) Pub. No.: US 2022/0152148 A1
(43) Pub. Date: May 19, 2022

(54) MODULATION OF TYPE 2 IMMUNITY BY TARGETING CLEC-2 SIGNALING

(71) Applicants: The Broad Institute, Inc., Cambridge, MA (US); Massachusetts Institute of Technology, Cambridge, MA (US); The Brigham and Women's Hospital, Inc., Boston, MA (US)

(72) Inventors: Patrick R. Burkett, Boston, MA (US); Se-Jin Kim, Boston, MA (US); Samantha J. Riesenfeld, Cambridge, MA (US); Antonia Wallrapp, Boston, MA (US); Vijay K. Kuchroo, Boston, MA (US); Aviv Regev, Cambridge, MA (US)

(21) Appl. No.: 17/440,226

(22) PCT Filed: Mar. 18, 2020
(86) PCT No.: PCT/US2020/023387

§ 371 Date: Sep. 17, 2021

Related U.S. Application Data
(60) Provisional application No. 62/820,194, filed on Mar. 18, 2019.

(51) Int. Cl. A61K 38/17; A61P 29/00; G01N 33/50; G01N 33/569

(52) U.S. Cl. CPC A61K 38/177; A61K 38/1735; G01N 33/56972; G01N 33/5055; A61P 29/00

(57) ABSTRACT

Interleukin (IL)-33 is a critical regulator of allergic airway inflammation in the lung and is released by stressed or damaged epithelial cells. Here, Applicants show that alveolar macrophages regulate epithelial alarmin expression via CLEC-2 (C-type Lectin-like Receptor-2), which binds to PDPN (podoplanin). Therefore, CLEC-2/PDPN interactions are critical for regulating type 2 immunity in the lung and modulating expression of the epithelial alarmin IL-33. Methods are disclosed for therapeutic and screening applications. Novel therapeutic targets in alveolar macrophages and epithelial cells are disclosed.

### CROSS-REFERENCE TO RELATED APPLICATIONS

This application claims the benefit of U.S. Provisional Application No. 62/820,194, filed Mar. 18, 2019. The entire contents of the above-identified application are hereby fully incorporated herein by reference.

### STATEMENT REGARDING FEDERALLY SPONSORED RESEARCH

This invention was made with government support under Grant Nos. AI123516 and AI139536 granted by the National Institutes of Health. The government has certain rights in the invention.

### REFERENCE TO AN ELECTRONIC SEQUENCE LISTING

The contents of the electronic sequence listing (BROD_4130WP_ST25.txt”; Size is 8 Kilobytes and it was created on Mar. 18, 2020) is herein incorporated by reference in its entirety.

### TECHNICAL FIELD

The subject matter disclosed herein is generally directed to modulating type 2 inflammatory responses by modulation of CLEC-2 and CLEC-2/PDPN signaling.

### BACKGROUND

Asthma is a common condition driven by persistent dysregulated airway inflammation. Although advances in therapy have resulted in improved disease control for many patients, up to 10% of asthma patients continue to have difficult to control asthma characterized by a poor response to current therapies, highlighting the need for further investigation of molecular pathways that regulate airway inflammation. Recent research has highlighted that cytokines produced by stressed or damaged epithelial cells, including interleukin (IL)-33, IL-25, and TSLP, play a critical role in promoting tissue-based type 2 immune responses (1, 2). These cytokines, termed alarmins, activate innate immune mechanisms and amplify and promote the differentiation of antigen-specific CD4 T cells into different T helper (Th) subsets. Therapeutics targeting different alarmins are in clinical development for treating allergic diseases, and several have shown promise in the treatment of asthma in early stage clinical trials (3, 4). Despite the clear importance of alarmins in inducing type 2 immune responses in vivo, however, the mechanisms that regulate their production in tissues are still being elucidated.

Alveolar macrophages play a crucial role in promoting tissue homeostasis of the alveolus and distal airways. Under homeostatic conditions alveolar macrophages are typically anti-inflammatory (5-8). However, given their position at the interface of the lung and the outside environment, they also play an important role in promoting immune responses to inhaled irritants and pathogens (9, 10). The balance between promoting homeostatic versus inflammatory responses is in part mediated by close cellular interactions between alveolar macrophages and the epithelial lining of the lung. Additionally, lung epithelial cells produce several cytokines critical to the maturation and maintenance of alveolar macrophages, including GM-CSF and TGFβ (7, 11). Alveolar macrophages have been noted to adopt a pro-inflammatory phenotype in several chronic inflammatory lung diseases, including asthma, suggesting that aberrant macrophage responses may contribute to disease pathogenesis, but the pathways that regulate this remain unclear (12).

### SUMMARY

In one aspect, the present invention provides for a method of modulating a Type 2 and/or type 3 inflammatory response in a subject in need thereof comprising administering to the subject a therapeutically effective amount of one or more agents capable of modulating CLEC-2 signaling. In certain embodiments, the Type 2 inflammatory response is an IL-33 mediated response. In certain embodiments, the one or more agents modulate the interaction of CLEC-2 with PDPN.

In certain embodiments, the interaction is blocked, whereby a Type 2 and/or Type 3 inflammatory immune response is enhanced. In certain embodiments, the one or more agents is a recombinant PDPN protein or protein fragment. In certain embodiments, the recombinant PDPN fragment comprises the extracellular domain of PDPN. In certain embodiments, the recombinant PDPN is modified to be more stable. In certain embodiments, the recombinant PDPN is a Fc fusion protein. In certain embodiments, the one or more agents is a recombinant CLEC-2 protein or protein fragment. In certain embodiments, the recombinant CLEC-2 fragment comprises the extracellular domain of CLEC-2. In certain embodiments, the recombinant CLEC-2 is a Fc fusion protein.

In certain embodiments, the one or more agents is a CLEC-2 signaling agonist, whereby a Type 2 inflammatory immune response is suppressed or homeostasis is maintained. In certain embodiments, the CLEC-2 agonist increases expression or activity of CLEC-2 in macrophages and/or monocytes. In certain embodiments, the agonist is a CLEC-2 expression vector targeted to macrophages and/or monocytes, wherein the agent is under control of a macrophage and/or monocyte specific promoter. In certain embodiments, the CLEC-2 agonist binds to CLEC-2. In certain embodiments, the one or more agents is a PDPN agonist, whereby a Type 2 inflammatory immune response is suppressed or homeostasis is maintained. In certain embodiments, the PDPN agonist binds to PDPN.

In certain embodiments, the one or more agents modulate the expression, activity, and/or function of one or more genes or gene products differentially expressed upon deletion of CLEC-2 in macrophages or absence of CLEC-2 in macrophages. In certain embodiments, the one or more genes are selected from Table 2 or the group consisting of Mthfd2, Itga4, L1cam, 9130019O22Rik, Gal, Clec1b, Mgst3, Tgfb2, Serpina3f, Ifi47, Vcam1, Gbp6, Slamf8, Best1, H2-Q9, Ccr5, Rgs12, H2-DMb1, Tgtp2, Gbp4, Pla2g7, Plxnb2, Cd5l, Enpp5, Ifitm3, Pbx1, Pla2g2d, Tgtp1, AA467197, Ptpro, H2-Q4, Irf5, C3, Mt2, Sqle, Ch25h, Il1a, Rilpl2, Igf1, Cd52, Sc4 mol, Smpdl3b, Fbp1, Pilrb2, C1qa, H2-M2, Cdh1, Cyp1a1, Ccl9, Marco, Plxna1, Cxcl16, Trpm2, Ccdc80 and Cd300a. In certain embodiments, the one or more genes are upregulated upon deletion of CLEC-2 and the one or more agents inhibit the one or more upregulated genes. In certain embodiments, the one or more upregulated genes are selected from the group consisting of: Plxnb2, Sqle, H2-Q9, Tgtp1, Cdh1, H2-M2, Ccdc80, C3, Cd300a, Slamf8, Mt2, Serpina3f, Cd52, Rilpl2, Enpp5, Rgs12, Pla2g7, Gbp4, Irf5, Best1, H2-Q4, Ccl9, Ccr5, Igf1, Cxcl16, C1qa, Trpm2, Vcam1, Fbp1, Ch25h, Marco, AA467197, Pla2g2d, Smpdl3b, H2-DMb1, Gbp6, Tgtp2, Ifi47, Il1a, Sc4 mol, Cyp1a1, Pilrb2, Plxna1, Ifitm3, Pbx1, Cd5l, Ptpro, Ctla4, Ptgs1 and Mmab.

In certain embodiments, the one or more agents target alveolar macrophages and/or monocytes. In certain embodiments, the one or more agents are targeted by a macrophage and/or monocyte specific expression vector, wherein the agent is under control of a macrophage and/or monocyte specific promoter.

In certain embodiments, the one or more agents comprise an antibody, small molecule, small molecule degrader, genetic modifying agent, antibody-like protein scaffold, aptamer, protein, or any combination thereof. In certain embodiments, the antibody is a CLEC-2 or PDPN antibody. In certain embodiments, the genetic modifying agent comprises a CRISPR system, RNAi system, a zinc finger nuclease system, a TALE, or a meganuclease. In certain embodiments, the CRISPR system is a Class 1 or Class 2 CRISPR system. In certain embodiments, the Class 2 system comprises a Type II Cas polypeptide. In certain embodiments, the Type II Cas is a Cas9. In certain embodiments, the Class 2 system comprises a Type V Cas polypeptide. In certain embodiments, the Type V Cas is Cas12a, Cas12b, Cas12c, Cas12d (CasY), Cas12e(CasX), or Cas14. In certain embodiments, the Class 2 system comprises a Type VI Cas polypeptide. In certain embodiments, the Type VI Cas is Cas13a, Cas13b, Cas13c or Cas13d. In certain embodiments, the CRISPR system comprises a dCas fused or otherwise linked to a nucleotide deaminase. In certain embodiments, the nucleotide deaminase is a cytidine deaminase or an adenosine deaminase. In certain embodiments, the dCas is a dCas9, dCas12 or dCas13. In certain embodiments, the CRISPR system is a prime editing system.

In certain embodiments, the treatment is administered to a mucosal surface. In certain embodiments, the treatment is administered to the lung, nasal passage (e.g., intranasally), trachea, gut, intestine, or esophagus. In certain embodiments, the treatment is administered by aerosol inhalation. In certain embodiments, the treatment is administered by a time release composition.

In certain embodiments, the subject is suffering from or at risk for an allergic inflammatory disease. In certain embodiments, the allergic inflammatory disease is selected from the group consisting of asthma, allergy, allergic rhinitis, allergic airway inflammation, atopic dermatitis (AD), chronic obstructive pulmonary disease (COPD), inflammatory bowel disease (IBD), multiple sclerosis, arthritis, psoriasis, eosinophilic esophagitis, eosinophilic pneumonia, eosinophilic psoriasis, hypereosinophilic syndrome, graft-versus-host disease, uveitis, cardiovascular disease, pain, multiple sclerosis, lupus, vasculitis, chronic idiopathic urticaria and Eosinophilic Granulomatosis with Polyangiitis (Churg-Strauss Syndrome). In certain embodiments, the asthma is selected from the group consisting of allergic asthma, non-allergic asthma, severe refractory asthma, asthma exacerbations, viral-induced asthma or viral-induced asthma exacerbations, steroid resistant asthma, steroid sensitive asthma, eosinophilic asthma and non-eosinophilic asthma. In certain embodiments, the allergy is to an allergen selected from the group consisting of food, pollen, mold, dust mites, animals, and animal dander. In certain embodiments, IBD comprises a disease selected from the group consisting of ulcerative colitis (UC), Crohn's Disease, collagenous colitis, lymphocytic colitis, ischemic colitis, diversion colitis, Behcet's syndrome, infective colitis, indeterminate colitis, and other disorders characterized by inflammation of the mucosal layer of the large intestine or colon. In certain embodiments, the arthritis is selected from the group consisting of osteoarthritis, rheumatoid arthritis and psoriatic arthritis.

In another aspect, the present invention provides for a method of screening for agents capable of shifting alveolar macrophages to a homeostatic or inflammatory macrophage comprising contacting macrophages with one or more agents and detecting expression of one or more genes selected from Table 2.

In another aspect, the present invention provides for a method of detecting a Type 2 and/or type 3 inflammatory state in a subject, comprising detecting in immune cells obtained from the subject the expression or activity of one or more genes selected from Table 2; or the group consisting of Plxnb2, Sqle, H2-Q9, Tgtp1, Cdh1, H2-M2, Ccdc80, C3, Cd300a, Slamf8, Mt2, Serpina3f, Cd52, Rilpl2, Enpp5, Rgs12, Pla2g7, Gbp4, Irf5, Best1, H2-Q4, Ccl9, Ccr5, Igf1, Cxcl16, C1qa, Trpm2, Vcam1, Fbp1, Ch25h, Marco, AA467197, Pla2g2d, Smpdl3b, H2-DMb1, Gbp6, Tgtp2, Ifi47, Il1a, Sc4 mol, Cyp1a1, Pilrb2, Plxna1, Ifitm3, Pbx1, Cd5l, Ptpro, Ctla4, Ptgs1 and Mmab; or the group consisting of Mthfd2, Itga4, L1cam, 9130019O22Rik, Gal, Clec1b, Mgst3, Tgfb2, Serpina3f, Ifi47, Vcam1, Gbp6, Slamf8, Best1, H2-Q9, Ccr5, Rgs12, H2-DMb1, Tgtp2, Gbp4, Pla2g7, Plxnb2, Cd5l, Enpp5, Ifitm3, Pbx1, Pla2g2d, Tgtp1, AA467197, Ptpro, H2-Q4, Irf5, C3, Mt2, Sqle, Ch25h, Il1a, Rilpl2, Igf1, Cd52, Sc4 mol, Smpdl3b, Fbp1, Pilrb2, C1qa, H2-M2, Cdh1, Cyp1a1, Ccl9, Marco, Plxna1, Cxcl16, Trpm2, Ccdc80 and Cd300a, wherein upregulation of upregulated genes in Table 2 and downregulation of downregulated genes in Table 2 indicate an inflammatory state. In certain embodiments, the immune cell is a macrophage.

In another aspect, the present invention provides for a method of treatment comprising detecting a Type 2 and/or type 3 inflammatory state in a subject comprising detecting in immune cells from a subject to be treated the one or more genes of claim 46; and administering to the subject the treatment of any one of claims 1-43; and/or a standard anti-inflammatory treatment if the one or more genes are detected. In certain embodiments, the immune cell is a macrophage.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-FIG. 1E—Spontaneous airway inflammation in CLEC-2^{-/-} (KO) mice. (A, B) PAS-stained lung sections demonstrate spontaneous goblet cell hyperplasia and airway inflammation in CLEC-2^{-/-} mice. Representative images (A) and severity score (B) are shown. (C) Increased numbers of lung-infiltrating leukocytes in CLEC-2^{-/-} mice. (D) Spontaneous BAL eosinophilia in CLEC-2^{-/-} mice, as shown by flow cytometry. Summary data and representative flow cytometry plots (gated on CD11b^{hi} CD11c⁻ CD45⁺ cells) are shown. (E) Increased airways hyperreactivity in CLEC-2^{-/-} mice. Airway resistance was measured following methacholine challenge. For panels (B-D), each point represents an individual mouse. For graphs, the average±SEM is shown. *P<0.05; **P<0.01; ***P<0.001; ****P<0.0001 by unpaired two tailed t-test.

FIG. 2A-FIG. 2F—Dysregulated type 2 and 3 immunity in the lungs of CLEC-2^{-/-} (KO) mice. (A) Differentially expressed genes in lung-infiltrating leukocytes. Gene expression was analyzed by Nanostring. Only those genes with >1.5 fold changes in gene expression are shown. Each column represents an individual mouse. (B) The concentration of IL-33 was assessed in lung supernatants by ELISA. (C) Lung resident CD4 T cells from CLEC-2^{-/-} mice have a spontaneously activated phenotype. The frequency of CD69⁺ and CD44^{hi} CD4 T cells is shown. (D) The frequency of CD4 T cells positive for the indicated cytokine by intracellular cytokine staining is shown. (E) CD4 T cells were sorted and expression of the indicated genes was evaluated by qPCR. (F) Increased expression of ST2 and ICOS on lung resident CD4 T cells, as shown by flow cytometry. The frequency of cells expressing the indicated marker is shown. For panels (B-F) each data point represents an individual mouse. For graphs the average±SEM is shown. *P<0.05; **P<0.01; ***P<0.001; ****P<0.0001 by unpaired two tailed t-test (B-E); ns, not significant.

FIG. 3A-FIG. 3F—CLEC-2 is expressed by alveolar macrophages, and alveolar macrophage homeostasis is altered in the absence of CLEC-2. (A) The indicated lung-resident cell types were sorted by FACS and expression of Clec1b was evaluated by qPCR. (B,C) The frequency (B) and number (C) of alveolar macrophages (CD11c^{hi} CD11b^{lo} SiglecF⁺) in CLEC-2^{-/-} (KO) mice are reduced compared to controls (WT), as determined by flow cytometry. (D) MHC class II expression is increased on CLEC-2^{-/-} alveolar macrophages. A representative flow cytometry plot and summary graph showing the gMFI of MHC class II on alveolar macrophages are shown. (E) CLEC-2^{-/-} alveolar macrophages have an altered gene expression profile. Heatmap of expression, z-scored by row, of top differentially expressed genes (y axis), i.e., top 55 genes, ranked by p-value, exhibiting a fold change in expression of at least 1.5 between genotypes (x axis, each column represents an individual mouse) (P<0.05, generalized linear regression). The annotated genes: Gbp6, Irf5, Il1a and Marco are associated with an M1 phenotype, and Cd5l, AA467197, Ch25h and Cdh1 are associated with an M2 phenotype, H2-Q9, H2-DMb1, H2-Q4 and H2-M2 are associated with antigen presentation. Each column represents an individual mouse. (F) Expression of Arg1 in alveolar macrophages from CLEC-2^{-/-} or controls was determined by qPCR. Panel (A) is representative of two independent experiments. Each data point in panels (B-D, F) represents an individual mouse. For graphs the average±SEM is shown. *P<0.05; **P<0.01 by unpaired two tailed t-test.

FIG. 4A-FIG. 4C—CLEC-2^{-/-} alveolar macrophages amplify Th2 differentiation. Alveolar macrophages or lung-resident monocytes isolated from CLEC-2^{-/-} or control mice were co-cultured with wildtype naïve CD4 T cells under unbiased or Th2 conditions. (A) CLEC-2^{-/-} alveolar macrophages amplify Th2 cell proliferation in vitro. Proliferation was assessed by measuring CellTrace violet dilution. Summary data and representative flow cytometry plots are shown. (B) Increased expression of type 2 cytokines in Th2 cells cultured with CLEC-2^{-/-} alveolar macrophages. Expression of the indicated cytokines was determined by qPCR. (C) CLEC-2^{-/-} alveolar macrophages enhance IL-13 production by Th2 cells. The concentration of IL-13 in supernatants (left) or the frequency of IL-13⁺ CD4 T cells (right) are shown, quantified by Legendplex or intracellular cytokine staining. Panels (A-C) represent pooled data from two independent experiments. For graphs the average±SEM is shown. *P<0.05; **P<0.01; ***P<0.001 by one-way ANOVA (A) or unpaired two tailed t-test (B-C); ns, not significant.

FIG. 5A-FIG. 5G—Myeloid specific deletion of CLEC-2 results in dysregulated pulmonary type 2 responses. (A) Expression of the indicated cytokines in lung-resident leukocytes from CLEC-2^{fl/fl} LysM-Cre (circle), CLEC-2^{fl/fl} PF4-Cre (square), or CLEC-2^{fl/fl} littermates was assessed by qPCR. (B-F) CLEC-2^{fl/fl} LysM-Cre mice and CLEC-2^{fl/fl} littermates were given HDM to induce airway inflammation. (B, C) The frequency of BAL eosinophils and CD44^{hi} CD4 T cells is increased in CLEC-2^{fl/fl} LysM-Cre mice after HDM exposure. (D) Expression of the indicated cytokines in lung-resident leukocytes of CLEC-2^{fl/fl} LysM-Cre mice or Cre⁻ controls as assessed by qPCR is shown. (E) The number of alveolar macrophages in CLEC-2^{fl/fl} LysM-Cre mice or Cre⁻ controls is shown. (F,G) Expression of Il33 in lung tissue of either CLEC-2^{fl/fl} LysM-Cre mice (F) or PDPN^{fl/fl} Nkx2.1-Cre mice (G, triangle) compared to PDPN^{fl/fl} littermate controls was assessed by qPCR. Each data point represents one mouse, data is pooled from two independent experiments. For graphs the average±SEM is shown. *P<0.05; **P<0.01; ****P<0.0001 by unpaired two tailed t-test.

FIG. 6A-FIG. 6D—Blockade of endogenous CLEC-2/PDPN interactions enhances aero-allergen-induced type 2 responses. Mice were given intranasal HDM with either PDPN-Fc (circle) or isotype control (black circle). (A) Increased frequency of BAL eosinophils in mice treated with PDPN-Fc, compared to isotype treated controls. (B) Increased frequency of lung-resident ST2⁺ CD4 T cells after PDPN-Fc treatment. (C) Increase in CD4 T cells expressing IL-5 and IL-13 after PDPN-Fc treatment, as shown by intracellular cytokine staining. (D) Increase in expression of the indicated cytokines in the lungs after PDPN-Fc treatment, as shown by qPCR. Each data point represents one mouse, data is pooled from two independent experiments. For graphs the average±SEM is shown. *P<0.05; **P<0.01; ***P<0.001; ****P<0.0001 by unpaired two tailed t-test.

FIG. 7A-FIG. 7H—Dysregulated IL-33 expression drives spontaneous type 2 immunity in CLEC-2^{-/-} mice. (A) Representative lung histology of wildtype, ST2^{-/-} (ST2 KO), CLEC-2^{-/-} (CLEC2 KO), and CLEC-2^{-/-} ST2^{-/-} mice (CLEC2/ST2 DKO). (B) BAL eosinophil frequency in CLEC-2^{-/-} mice is reduced in the absence of ST2. (C) The frequency of lung-resident CD44^{hi} CD4 T cells is reduced in CLEC-2^{-/-} ST2^{-/-} mice compared to CLEC-2^{-/-} mice. (D) Alveolar macrophage numbers are increased in CLEC-2^{-/-} ST2^{-/-} mice compared to CLEC-2^{-/-} mice. (E) Expression of the indicated cytokines in lung-resident leukocytes was assessed by qPCR. (F) Differential gene expression in the lungs of WT, ST2^{-/-}, CLEC-2^{-/-}, and CLEC-2^{-/-} ST2^{-/-} mice. Expression of the indicated genes was assessed by Nanostring. (G,H) Expression of Il1a (G), and Arg1 (H) in lung-resident leukocytes was assessed by qPCR. For panels (B-E) each data point represents an individual mouse. Data are pooled from multiple experiments. For graphs the average±SEM is shown. *P<0.05; **P<0.01; ***P<0.001; ****P<0.0001 by unpaired two tailed t-test (D) or one-way ANOVA (B, C, E, G, H).

FIG. 8A-FIG. 8F—Type 2 and type 3 immunity is dysregulated in the absence of CLEC-2. (A) MHC class II⁺ monocytes are increased in the lungs of CLEC-2^{-/-} (KO) mice. The number of MHC class II⁺ monocytes (MHC class II⁺, Ly6C^{hi}, Ly6G⁻, CD11b⁺) was determined by flow cytometry, and degree of MHC class II expression (as measured by gMFI of MHC class II) is shown. (B) Expression of indicated cytokines and transcription factors in lung-infiltrating cells was assessed by qPCR. (C) Altered cytokine milieu in the lungs of CLEC-2^{-/-} mice. Levels of the indicated cytokines in lung supernatant were analyzed using LegendPlex. (D) CD4 T cells were sorted from the lungs of CLEC-2^{-/-} mice or controls and expression of the indicated genes was evaluated by qPCR. (E) Increased expression of ST2 and ICOS on CD4 T cells in the BAL of CLEC-2^{-/-} mice, as determined by flow cytometry. (F) Increased gene expression of Il1rl1 by CD4 T cells sorted from CLEC-2^{-/-} mice compared to controls was assessed by qPCR. For all panels each data point reflects one individual mouse. For all panels the average±SEM is shown. *P<0.05; **P<0.01; ***P<0.001; ****P<0.0001 by unpaired two tailed t-test (A-E) or two-way ANOVA (C); ns, not significant.

FIG. 9A-FIG. 9D—Altered gene expression in CLEC-2^{-/-} myeloid cells. (A) CLEC-2 is preferentially expressed by alveolar macrophages, compared to monocytes. The indicated cell types were sorted from the lungs of CLEC-2^{-/-} mice or controls and expression of CLEC-2 (Clec1b) was assessed by qPCR. (B) Transcriptional profiling of CLEC-2^{-/-} alveolar macrophages highlights a mixed M1/M2 activation profile. Log 2FC for those genes significantly differentially expressed between CLEC-2^{-/-} (nn) versus CLEC-2^{+/-} (pn) alveolar macrophages (X-axis) as well between either M1 (left) or M2 (right) versus M0 BMDMs (Y-axis) is shown (23). (C) Clec1b expression in WT or CLEC-2^{-/-} (KO) BMDMs was determined by qPCR. (D) Increased Arg1 expression in CLEC-2^{-/-} BMDMs. Arg1 expression was determined by qPCR in either control or IL-4 stimulated BMDMs. For panel (A, C) data are representative of two independent experiments. For panel (D) data represent individual replicates. Mean±SEM is shown for panel (A, C, D), ****P<0.0001 by one-way ANOVA.

FIG. 10A-FIG. 10E—CLEC-2^{-/-} alveolar macrophages potentiate Th2 differentiation. (A) Increased IL-2 production by CD4 T cells cultured with CLEC-2^{-/-} alveolar macrophages. The concentration of IL-2 in culture supernatants was determined by LegendPlex. (B, C) Cytokine expression under non-biased conditions. After co-culture of CD4 T cells with either CLEC-2^{-/-} or CLEC-2^{+/-} alveolar macrophages, the expression of the indicated cytokines was assessed by qPCR. (D) CLEC-2^{-/-} monocytes do not significantly enhance Th2 differentiation in vitro. Cells were cultured with naïve CD4 T cells and expression of IL-13 assessed by intracellular cytokine staining. (E) CLEC-2^{-/-} (KO) BMDMs potentiate Th2 differentiation. CD4 T cells were cultured with the indicated BMDMs and cytokine expression assessed by qPCR. Panels (A-E) show pooled data from at least two independent experiments. Each data point reflects an individual replicate. Mean±SEM is shown, *P<0.05; **P<0.01; ***P<0.001 by unpaired two tailed t-test (A-E); ns, not significant.

FIG. 11A-FIG. 11G—Cell type specific role of CLEC-2. (A) Expression of Clec1b was assessed in the indicated cell types sorted from either CLEC-2^{fl/fl} LysM-Cre or CLEC-2^{fl/fl} mice. Data is representative of two independent experiments. (B) Baseline eosinophil frequency and number in the BAL of either CLEC-2^{fl/fl} LysM-Cre (circles) or CLEC-2^{fl/fl} PF4-Cre (squares) mice, in comparison to CLEC-2^{fl/fl} littermate controls, as determined by flow cytometry. (C-E) Similar magnitude of type 2 and type 3 responses after HDM administration in CLEC-2^{fl/fl} PF4-Cre mice. (C) Expression of the indicated cytokines in lung infiltrating leukocytes in CLEC-2^{fl/fl} PF4-Cre mice or Cre⁻ controls was assessed by qPCR. (D) Expression of the indicated cytokines in CD4 T cells from the lungs of CLEC-2^{fl/fl} PF4-Cre mice or Cre⁻ controls was assessed by intracellular cytokine staining. (E) The frequency of eosinophils in the BAL in CLEC-2^{fl/fl} PF4-Cre mice or Cre⁻ controls after HDM challenge was determined by flow cytometry. (F) Trend towards higher expression of I/rl1 at the RNA (left) in lung and protein level (right) in CD4 T cells from CLEC-2^{fl/fl} LysM-Cre mice and CLEC-2^{fl/fl} nm littermates. (G) Frequency of lung infiltrating eosinophils after HDM treatment in mice given either PDPN-Fc or isotype control was determined by flow cytometry. For panels (B-G) each data point represents an individual mouse. Mean±SEM is shown, *P<0.05 by unpaired two tailed t-test.

FIG. 12A-FIG. 12B—Compensatory increases in alarmin receptor expression in CLEC2/ST2 DKO mice. Expression of (A) Il17rb and (B) Crlf2 in the lungs of the indicated mice was assessed by qPCR. Each data point represents an individual mouse. Mean±SEM is shown, *P<0.05; **P<0.01; ***P<0.001 by unpaired two tailed t-test.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide methods and compositions for modulating type 2 immune responses. Embodiments disclosed herein also provide methods of screening for modulating agents that can be used to modulate type 2 immune responses. In certain embodiments, the agents are screened in alveolar macrophages.

Given the close association between alveolar macrophages and epithelial cells, Applicants hypothesized that alterations in signaling between these two cell types could modify alarmin production and thereby modulate allergic airway inflammation.

Previously it has been shown that PDPN^{-/-} mice that survive to adulthood develop spontaneous mononuclear cell infiltrates in multiple organs, including the lungs, highlighting the importance of PDPN in regulating tissue inflammation (13). Moreover, PDPN is an inhibitory molecule when expressed on effector T cells, where it promotes T cell exhaustion and inhibits T cell survival in a CLEC-2-dependent manner (13, 14). However, both PDPN and CLEC-2 are expressed by multiple cell types, and the role of CLEC-2 and PDPN expression in regulating tissue homeostasis and function in other contexts has not been elucidated (15). Notably, PDPN is highly expressed in pulmonary epithelial cells, and Applicants find that CLEC-2 is preferentially expressed by alveolar macrophages, compared to other lung-resident myeloid cells, suggesting a potential role for CLEC-2/PDPN-mediated interactions between alveolar macrophages and epithelial cells in modulating immune responses in the lung. Here, Applicants specifically identify a role for CLEC-2 in regulating both alveolar macrophage function and allergic airway inflammation, in part by controlling expression of the alarmin IL-33 and potentiating Th2 proliferation and differentiation.

Interleukin (IL)-33 is a critical regulator of type 2 inflammation in the lung and is released by stressed or damaged epithelial cells. Here, Applicants identify that alveolar macrophages express C-type Lectin-like Receptor-2 (CLEC-2), while its ligand, podoplanin (PDPN), is known to be expressed by lung epithelial cells. Applicants find that CLEC-2^{-/-} mice develop spontaneous airway inflammation and have increased expression of IL-33. Alveolar macrophages are reduced in frequency and number in CLEC-2^{-/-} mice but display an activated phenotype and enhance Th2 differentiation upon co-culture with T cells. Myeloid-specific deletion of CLEC-2 enhances type 2 responses after allergen challenge, highlighting the importance of myeloid cell-specific CLEC-2 expression in regulating adaptive type 2 responses. IL-33 expression was increased after allergen challenge in mice with either myeloid-specific CLEC-2 deletion, epithelial cell deletion of PDPN, and by blockade of endogenous CLEC-2/PDPN interactions. Spontaneous airway inflammation in CLEC-2^{-/-} mice is ameliorated by deletion of the IL-33 receptor, ST2. Therefore, myeloid cell expression of CLEC-2 is critical for regulating type 2 immunity in the lung, in part by controlling expression of the epithelial alarmin IL-33. Applicants also identified genes upregulated and downregulated in alveolar macrophages as a result of CLEC-2 knockout. Thus, CLEC-2, PDPN and genes regulated by CLEC-2 in macrophages are targets useful in therapeutic, diagnostic and screening applications.

### Expression Signatures

In certain example embodiments, the therapeutic, diagnostic, and screening methods disclosed herein target, detect, or otherwise make use of one or more biomarkers of an expression signature. As used herein, the term “biomarker” can refer to a gene, an mRNA, cDNA, an antisense transcript, a miRNA, a polypeptide, a protein, a protein fragment, or any other nucleic acid sequence or polypeptide sequence that indicates either gene expression levels or protein production levels. Accordingly, it should be understood that reference to a “signature” in the context of those embodiments may encompass any biomarker or biomarkers whose expression profile or whose occurrence is associated with a specific cell type, subtype, or cell state of a specific cell type or subtype within a population of cells (e.g., inflammatory or homeostatic macrophages, Th2 cells) or a specific biological program. As used herein, the term “module” or “biological program” can be used interchangeably with “expression program” and refers to a set of biomarkers that share a role in a biological function (e.g., an activation program, cell differentiation program, proliferation program). Biological programs can include a pattern of biomarker expression that result in a corresponding physiological event or phenotypic trait. Biological programs can include up to several hundred biomarkers that are expressed in a spatially and temporally controlled fashion. Expression of individual biomarkers can be shared between biological programs. Expression of individual biomarkers can be shared among different single cell types; however, expression of a biological program may be cell type specific or temporally specific (e.g., the biological program is expressed in a cell type at a specific time). Expression of a biological program may be regulated by a master switch, such as a nuclear receptor or transcription factor.

In certain embodiments, the expression of the signatures disclosed herein (e.g., inflammatory or homeostatic macrophage signatures) is dependent on epigenetic modification of the biomarkers or regulatory elements associated with the signatures (e.g., chromatin modifications or chromatin accessibility). Thus, in certain embodiments, use of signature biomarkers includes epigenetic modifications of the biomarkers that may be detected or modulated. As used herein, the terms “signature”, “expression profile”, or “expression program” may be used interchangeably (e.g., expression of genes, expression of gene products or polypeptides). It is to be understood that also when referring to proteins (e.g. differentially expressed proteins), such may fall within the definition of “gene” signature. Levels of expression or activity may be compared between different cells in order to characterize or identify, for instance, signatures specific for cell (sub)populations. Increased or decreased expression or activity or prevalence of signature biomarkers may be compared between different cells in order to characterize or identify for instance specific cell (sub)populations. The detection of a signature in single cells may be used to identify and quantitate for instance specific cell (sub)populations. A signature may include a biomarker whose expression or occurrence is specific to a cell (sub)population, such that expression or occurrence is exclusive to the cell (sub)population. An expression signature as used herein may thus refer to any set of up- and/or down-regulated biomarkers that are representative of a cell type or subtype. An expression signature as used herein, may also refer to any set of up- and/or down-regulated biomarkers between different cells or cell (sub)populations derived from a gene-expression profile. For example, an expression signature may comprise a list of biomarkers differentially expressed in a distinction of interest.

The signature according to certain embodiments of the present invention may comprise or consist of one or more biomarkers, such as for instance 1, 2, 3, 4, 5, 6, 7, 8, 9, 10 or more. In certain embodiments, the signature may comprise or consist of two or more biomarkers, such as for instance 2, 3, 4, 5, 6, 7, 8, 9, 10 or more. In certain embodiments, the signature may comprise or consist of three or more biomarkers, such as for instance 3, 4, 5, 6, 7, 8, 9, 10 or more. In certain embodiments, the signature may comprise or consist of four or more biomarkers, such as for instance 4, 5, 6, 7, 8, 9, 10 or more. In certain embodiments, the signature may comprise or consist of five or more biomarkers, such as for instance 5, 6, 7, 8, 9, 10 or more. In certain embodiments, the signature may comprise or consist of six or more biomarkers, for instance 6, 7, 8, 9, 10 or more. In certain embodiments, the signature may comprise or consist of seven or more biomarkers, such as for instance 7, 8, 9, 10 or more. In certain embodiments, the signature may comprise or consist of eight or more biomarkers, such as for instance 8, 9, 10 or more. In certain embodiments, the signature may comprise or consist of nine or more biomarkers, such as for instance 9, 10 or more. In certain embodiments, the signature may comprise or consist of ten or more biomarkers, such as for instance 10, 11, 12, 13, 14, 15, or more. It is to be understood that a signature according to the invention may also include, for instance, different types of biomarkers combined (e.g. genes and proteins).

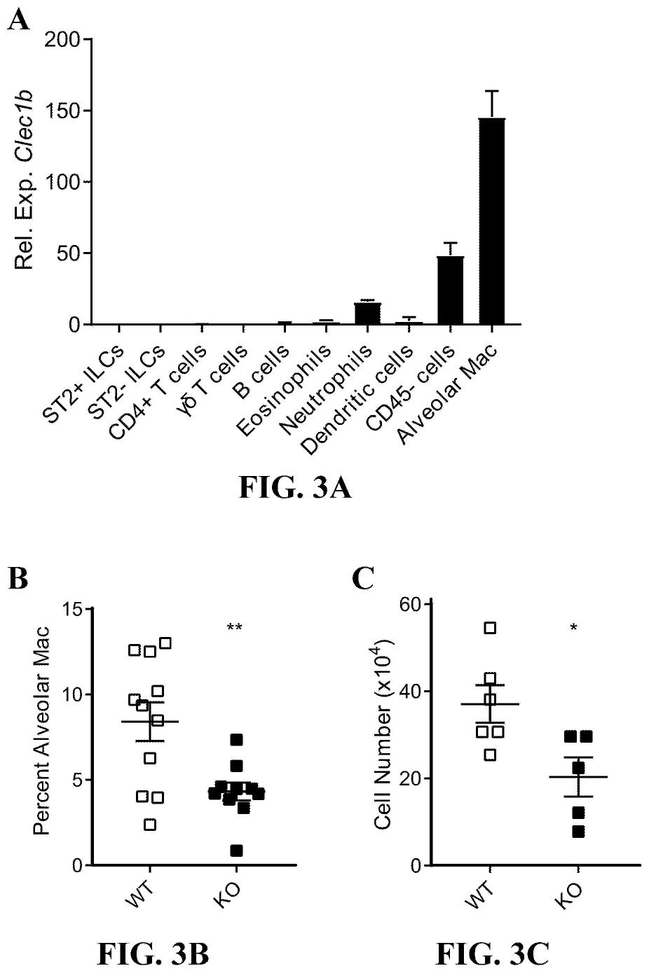

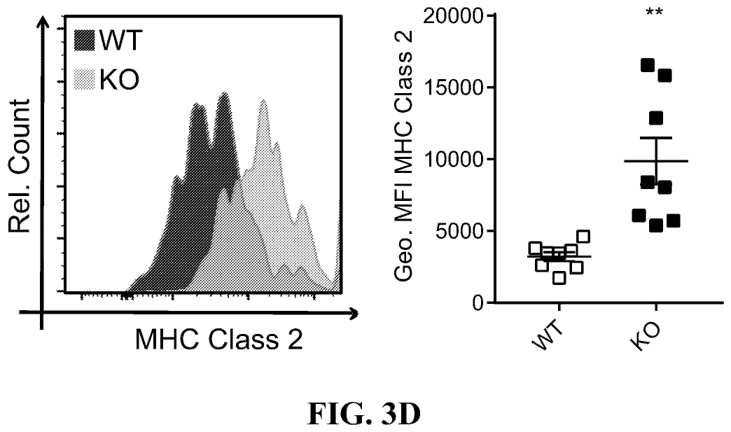

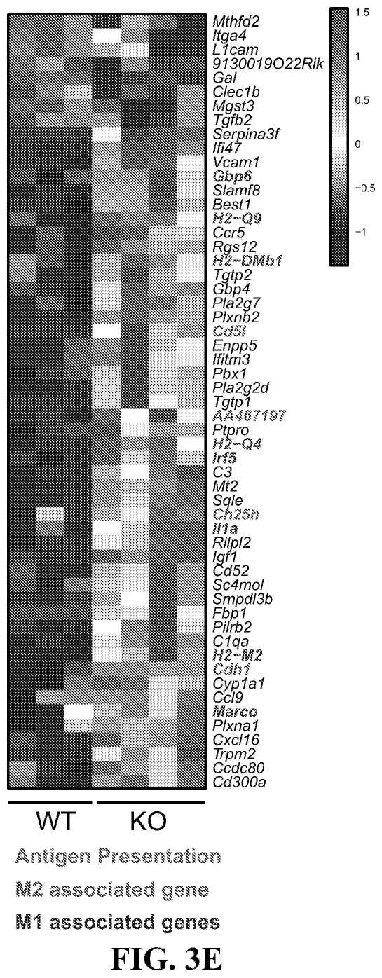

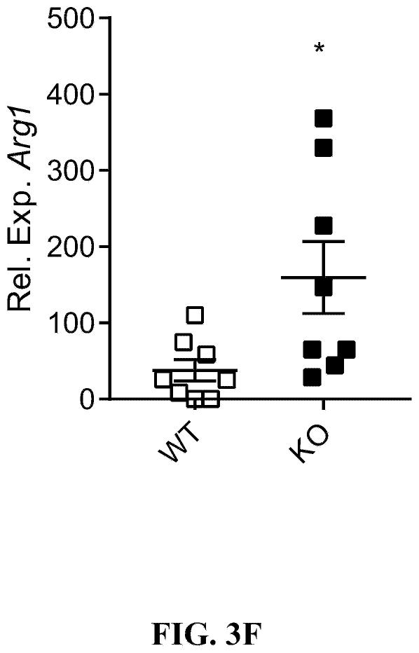

In certain embodiments, a signature is characterized as being specific for a particular cell or cell (sub)population if it is upregulated or only present, detected or detectable in that particular cell or cell (sub)population, or alternatively is downregulated or only absent, or undetectable in that particular cell or cell (sub)population. In this context, a signature consists of one or more differentially expressed genes/proteins or differential epigenetic elements when comparing different cells or cell (sub)populations, including comparing different immune cells or immune cell (sub)populations (e.g., macrophages), as well as comparing immune cells or immune cell (sub)populations with other immune cells or immune cell (sub)populations. It is to be understood that “differentially expressed” biomarkers include biomarkers which are up- or down-regulated as well as biomarkers which are turned on or off. When referring to up-or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art. Differential expression of biomarkers may also be determined by comparing expression of biomarkers in a population of cells or in a single cell. In certain embodiments, expression of one or more biomarkers is mutually exclusive in cells having a different cell state or subtype (e.g., two genes are not expressed at the same time). In certain embodiments, a specific signature may have one or more biomarkers upregulated or downregulated as compared to other biomarkers in the signature within a single cell (see, e.g., FIG. 3E). Thus, a cell type or subtype can be determined by determining the pattern of expression in a single cell.

As discussed herein, differentially expressed biomarkers may be differentially expressed on a single cell level or may be differentially expressed on a cell population level. Preferably, the differentially expressed biomarkers as discussed herein, such as constituting the expression signatures as discussed herein, when as to the cell population level, refer to biomarkers that are differentially expressed in all or substantially all cells of the population (such as at least 80%, preferably at least 90%, such as at least 95% of the individual cells). This allows one to define a particular subpopulation of cells. As referred to herein, a “subpopulation” of cells preferably refers to a particular subset of cells of a particular cell type (e.g., macrophages) which can be distinguished or are uniquely identifiable and set apart from other cells of this cell type. The cell subpopulation may be phenotypically characterized and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferable is meant induction or alternatively suppression (or upregulation or downregulation) of at least one biomarker of the signature, such as for instance at least two, at least three, at least four, at least five, at least six, or all biomarkers of the signature.

Example gene signatures are further described below.

### Macrophage Inflammatory Gene Signature

In certain embodiments, a macrophage inflammatory gene signature comprises one or more biomarkers selected from Table 2 or a subset of genes selected from FIG. 3E. Genes that are upregulated or downregulated in CLEC-2 −/− macrophages represent genes in the inflammatory and homeostatic signature, because CLEC-2 expression has now been shown to be associated with the homeostatic cell state in macrophages. Therefore, the genes upregulated are upregulated in the inflammatory signature, the genes downregulated are downregulated in the inflammatory signature, the genes upregulated are downregulated regulated in the homeostatic signature, and the genes downregulated are upregulated in the homeostatic signature.

The highest ranked 50 genes that are upregulated in CLEC-2 −/− macrophages are Plxnb2, Sqle, H2-Q9, Tgtp1, Cdh1, H2-M2, Ccdc80, C3, Cd300a, Slamf8, Mt2, Serpina3f, Cd52, Rilpl2, Enpp5, Rgs12, Pla2g7, Gbp4, Irf5, Best1, H2-Q4, Ccl9, Ccr5, Igf1, Cxcl16, C1qa, Trpm2, Vcam1, Fbp1, Ch25h, Marco, AA467197, Pla2g2d, Smpdl3b, H2-DMb1, Gbp6, Tgtp2, Ifi47, Il1a, Sc4 mol, Cyp1a1, Pilrb2, Plxna1, Ifitm3, Pbx1, Cd5l, Ptpro, Ctla4, Ptgs1 and Mmab.

The highest ranked genes that are downregulated in CLEC-2 −/− macrophages are Gal, Clec1b, Mgst3, Tgfb2, L1cam, 9130019O22Rik, Mthfd2, Itga4, Fam214a, Itgad, Tsc22d3, Alppl2, Eps8, Heatr5a, Mlph, Zzef1, Xpc, Cspg4, Cdkn1b, Col6a1, Egfem1, Lamc2, Wdr13, Syne3, Pafah1b1, Ano1, Ncam2, Asns and Trp53i11.

The highest ranked 100 genes both up and down regulated are Plxnb2, Sqle, Gal, H2-Q9, Tgtp1, Cdh1, Clec1b, H2-M2, Mgst3, Ccdc80, C3, Cd300a, Slamf8, Mt2, Serpina3f, Cd52, Rilpl2, Enpp5, Rgs12, Pla2g7, Gbp4, Irf5, Best1, H2-Q4, Tgfb2, Ccl9, Ccr5, Igf1, L1cam, Cxcl16, C1qa, Trpm2, Vcam1, Fbp1, Ch25h, Marco, AA467197, Pla2g2d, Smpdl3b, H2-DMb1, Gbp6, Tgtp2, Ifi47, Il1a, 9130019O22Rik, Sc4 mol, Cyp1a1, Pilrb2, Plxna1, Mthfd2, Ifitm3, Itga4, Pbx1, Cd5l, Ptpro, Ctla4, Fam214a, Ptgs1, Mmab, Cyp27a1, H2-Q8, Fdps, Fads1, Itgad, Tsc22d3, Gdf15, Cxcr1, Alppl2, Wfdc17, Cyp51, Socs3, Hmox1, Plac8, Ciita, Gm12250, Ear11, Tspan32, Sema4d, Syngr1, Eps8, Neurl3, Lss, Tbc1d8, Bpifb1, Fdft1, Heatr5a, Igtp, Dgat1, Mlph, Asrgl1, Ccrl2, Zzef1, Xpc, Spint1, Aoah, Ly6i, Acer3, Cspg4, Beta-s and Ngfrap1. In certain embodiments, the signature includes a combination of up and down regulated genes.

In one example embodiment, the inflammatory signature comprises one gene from Table 2 and at least N additional biomarkers selected from Table 2 (e.g., Plxnb2 and one or more additional genes from Table 2), wherein N is equal to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

### Methods of Altering, Maintaining or Inducing Homeostasis of Macrophages

The following section provides multiple example embodiments for altering, maintaining or inducing homeostasis of macrophages. The methods may be administered to subjects at risk for having aberrant activation and or expansion of inflammatory macrophages leading to an aberrant type 2 immune response. Thus, the embodiments may be used to prevent and/or treat diseases and disorders characterized by aberrant type 2 immune responses. As used herein, type 2 immune response refers to a T helper type 2 (Th2) immune response, characterized by the production of interleukin-4 (IL-4), IL-5 and IL-13. Examples of diseases or disorders characterized by aberrant type 2 immune responses include, but are not limited to allergies (e.g., food allergies). As used herein “maintaining” means that if macrophages are at homeostasis, they prevent type 2 immune responses, and they are maintained in that current state (e.g., do not become inflammatory or do not prevent inflammatory responses). As used herein, “inducing homeostasis” means increasing the amount of homeostatic macrophages or switching inflammatory macrophages to homeostatic macrophages. The methods also provide for enhancing or inducing an inflammatory response. Thus, the embodiments may be used to treat a subject in need of an enhanced immune response (e.g., an infection, such as viral or bacterial).

### Modulations of CLEC-2/PDPN

In one aspect, CLEC-2 expressed on homeostatic macrophages interacts with PDPN expressed on epithelial cells and regulates expression of the alarmin IL-33 by the epithelial cells. In certain embodiments, modulation of the interaction can be used to regulate type 2 immune responses. In certain embodiments, agonists of CLEC-2 signaling or the interaction of CLEC-2 and PDPN can be used to block IL-33 production by epithelial cells to prevent or alleviate a type 2 immune response (e.g., asthma, airway inflammation, allergy). In certain embodiments, soluble forms of CLEC-2 or PDPN can be used to block the interaction and increase IL-33 production by epithelial cells to enhance a type 2 immune response. Agonists of CLEC-2 signaling may be PDPN protein, protein fragments, or functional variants. Antagonists of CLEC-2 signaling may be PDPN or CLEC-2 protein, protein fragments, or functional variants (e.g., PDPN-Fc, CLEC-2-Fc). In certain embodiments, CLEC-2 or PDPN antibodies can be used to block the interaction and increase IL-33 production by epithelial cells to enhance a type 2 immune response. Agonists of CLEC-2 signaling may be CLEC-2 or PDPN antibodies. Antagonists of CLEC-2 signaling may be CLEC-2 or PDPN antibodies.

As used herein, functional variant or fragment refers to peptides which peptide sequence differs from the amino acid sequence of the wild type protein, but that generally retains all the biological activity. Functional variants may also include modified peptides, fusion proteins (e.g., fused to another protein, polypeptide or the like, such as an immunoglobulin or a fragment thereof), or peptides having non-natural amino acids. Functional variants may have an extended residence time in body fluids. In certain embodiments, a variant has at least 80, 85, 90, 95, 99% of the biological activity. Preferably, a functional variant has at least 85%, at least 90%, at least 91%, at least 92%, at least 93%, at least 94%, at least 95%, at least 96%, at least 96%, at least 97%, at least 98%, at least 99% sequence identity.

As used herein, the term “functional fragments” refers to a specific peptide that has a biological activity of interest, which peptide sequence is a part of the peptide sequence of the reference peptide, and that can be of any length, provided the biological activity of peptide of reference is retained by said fragment.

CLEC-2, also known as CLEC1B, CLEC1B1, 1810061I13Rik, CLEC2, CLEC2B, PRO1384, QDED721, and C-type lectin domain family 1 member B, is C-type lectin-like receptor with a single cytoplasmic YXXL sequence known as a hem-immunoreceptor tyrosine-based activation motif (hemITAM). As used herein, CLEC-2 may refer to nucleotide or protein sequence according to accession numbers NM_001099431.1, NM_016509.3, NP_001092901.1, NP_057593.3, NM_001204253.1, NM_001204239.1, NM_019985.3, NP_001191182.1, NP_001191168.1 and NP_064369.1.

Podoplanin is a protein that in humans is encoded by the “PDPN” gene and is also known as PDPN, AGGRUS, GP36, GP40, Gp38, HT1A-1, OTS8, PA2.26, T1A, T1A-2, T1A2, and TI1A. As used herein, PDPN may refer to nucleotide or protein sequence according to accession numbers NM_001006625.1, NM_001006624.1, NM_198389.2, NM_006474.4, NM_010329.3, NM_001290822.1, NP_001006626.1, NP_001006625.1, NP_938203.2 and NP_006465.3.

Applicants provide for recombinant PDPN that can be used for blocking the interaction of CLEC-2 on macrophages and PDPN on epithelial cells to increase an inflammatory state (see examples). As used herein, the term “inflammatory state” refers to a cell state that is more susceptible to an inflammatory response as opposed to a homeostatic state (e.g., more susceptible to an inflammatory response caused by epithelial injury or stress) or to a cell state producing an inflammatory response. Recombinant PDPN comprising the extracellular domain (PDPN-Fc) has been shown to inhibit lymphangiogenesis and induce platelet aggregation (see, Cueni, et al. Podoplanin-Fc reduces lymphatic vessel formation in vitro and in vivo and causes disseminated intravascular coagulation when transgenically expressed in the skin. Blood. 2010; 116(20):4376-84). In certain embodiments, recombinant PDPN is PDPN-Fc. The present invention also provides for an agonist or mimic of podoplanin (PDPN). The agonist may be a soluble form of PDPN (see, e.g., WO2014043334A1).

Recombinant CLEC-2 can also be used for blocking the interaction of CLEC-2 on macrophages and PDPN on epithelial cells to increase an inflammatory state. Fc-CLEC-2 where the extracellular C-terminal domain is fused to a human Fc protein at its N-terminus and a dimeric human immunoglobulin Fc domain fusion protein (hCLEC-2-hFc2) have been described (see, e.g., WO2012174534A2; Wu, et al., Soluble CLEC2 Extracellular Domain Improves Glucose and Lipid Homeostasis by Regulating Liver Kupffer Cell Polarization, EBioMedicine. 2015 March; 2(3): 214-224; and Suzuki-Inoue, et al., Involvement of the snake toxin receptor CLEC-2, in podoplanin-mediated platelet activation, by cancer cells, J Biol Chem. 2007 Sep. 7; 282(36):25993-6001). In certain embodiments, recombinant CLEC-2 fused to one or more Fc proteins is used to modulate an immune response.

In certain embodiments, a CLEC-2 signaling agonist increases the expression or activity of CLEC-2 in macrophages and/or monocytes. In certain embodiments, during an inflammatory cell state, CLEC-2 expressing alveolar macrophages are replaced by inflammatory monocytes, which do not highly express CLEC-2 and are rapidly recruited to sites of acute inflammation. Therefore, in this setting, tonic CLEC-2/PDPN signals would be diminished, resulting in increased expression of IL-33 after epithelial injury or stress. In certain embodiments, increased expression of CLEC-2 in macrophages and/or monocytes can decrease expression of IL-33. Increased expression can be obtained by targeting CLEC-2 regulatory sequences (e.g., CRISPRa) or by introducing a vector for expressing exogenous CLEC-2. In certain embodiments, CLEC-2 signaling agonists modulate expression of downstream targets of CLEC-2 signaling, described further herein. Downstream targets may be modulated by a genome modifying agent, described further herein. The genome modifying agent may be introduced by a vector.

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Vectors for and that result in expression in a eukaryotic cell can be referred to herein as “eukaryotic expression vectors.” Common expression vectors of utility in recombinant DNA techniques are often in the form of plasmids. The vectors used herein may include viral vectors or plasmids. In preferred embodiments, viral vectors are used. In more preferred embodiments, lentiviral vectors are used.

In certain embodiments, the vectors used are tissue specific, in particular, specific to macrophages and/or monocytes. Tissue specific expression can be accomplished using tissue specific promoters. Promoters specific for macrophages useful for the present invention have been described and used in vivo (see, e.g., Kang et al., A macrophage-specific synthetic promoter for therapeutic application of adiponectin, Gene Therapy volume 21, pages 353-362 (2014); and Ahsan and Gore, Comparative analysis of macrophage associated vectors for use in genetic vaccine, Genet Vaccines Ther. 2011; 9: 10).

In certain embodiments, CLEC-2 signaling agonists or antagonists can include CLEC-2 or PDPN antibodies. In the case of CLEC2 antibodies, a neutralizing antibody will inhibit signaling through the CLEC2 pathway by either binding the CLEC2 receptor and preventing ligand binding to the receptor or by binding the ligand and prevent it from binding to the CLEC2 receptor. In the case of PDPN antibodies, a neutralizing antibody will inhibit signaling through the CLEC2 pathway by binding the ligand and prevent it from binding to the CLEC2 receptor. Antibodies are described further herein. Antibody-mediated targeting of platelet C-type lectin like receptor 2 (CLEC-2) has been shown to induce receptor downregulation and thrombocytopenia (see, e.g., Lorenz, et al. Targeted downregulation of platelet CLEC-2 occurs through Syk-independent internalization., Blood, 2015 125(26):4069-4077). In certain embodiments, CLEC-2 antibodies are administered to a mucosal surface, thus preventing adverse effects of systemic administration.

### Modulation of Macrophage Gene Signatures

In certain embodiments, macrophages characterized by expression of CLEC-2 regulate type 2 immune responses and are homeostatic macrophages. In certain embodiments, the macrophages are further characterized by expression of a signature described herein. In certain embodiments, modulation of one or more genes in the gene signature can shift macrophages to be homeostatic or inflammatory. In certain embodiments, one or more agents targeting one or more of the genes or gene products in the signature can be used to treat an aberrant immune response in a subject or prevent an inflammatory immune response in a subject at risk for an aberrant immune response (e.g., a subject with known allergies). In another example embodiment, a method of maintaining or inducing homeostasis of macrophages comprises administering or more agents capable of modulating expression, activity, or function of one or more biomarkers of the macrophage inflammatory gene signature defined herein. In certain embodiments, target genes that are upregulated in the absence of CLEC-2 are inhibited to increase homeostasis. In certain embodiments, target genes that are upregulated in the absence of CLEC-2 are administered or enhanced to increase inflammation. In certain embodiments, target genes that are downregulated in the absence of CLEC-2 are inhibited to increase inflammation. In certain embodiments, target genes that are downregulated in the absence of CLEC-2 are administered or enhanced to increase homeostasis. In certain embodiments, recombinant protein is administered. In certain embodiments, secreted proteins up or downregulated in the absence of CLEC-2 are administered. Thus, the present invention provides for agonists and antagonists of one or more downstream effectors that result from PDPN/CLEC-2 signaling in alveolar macrophages.

### Modulation and Modulating Agents

As used herein, “modulating” or “to modulate” generally means either reducing or inhibiting the expression or activity of, or alternatively increasing the expression or activity of a target or antigen (e.g., CLEC-2, PDPN, or macrophage gene signature gene). In particular, “modulating” or “to modulate” can mean either reducing or inhibiting the activity of, or alternatively increasing a (relevant or intended) biological activity of, a target or antigen as measured using a suitable in vitro, cellular or in vivo assay (which will usually depend on the target involved), by at least 5%, at least 10%, at least 25%, at least 50%, at least 60%, at least 70%, at least 80%, at least 90%, or more, compared to activity of the target in the same assay under the same conditions but without the presence of an agent. An “increase” or “decrease” refers to a statistically significant increase or decrease respectively. For the avoidance of doubt, an increase or decrease will be at least 10% relative to a reference, such as at least 10%, at least 20%, at least 30%, at least 40%, at least 50%, at least 60%, at least 70%, at least 80%, at least 90%, at least 95%, at least 97%, at least 98%, or more, up to and including at least 100% or more, in the case of an increase, for example, at least 2-fold, at least 3-fold, at least 4-fold, at least 5-fold, at least 6-fold, at least 7-fold, at least 8-fold, at least 9-fold, at least 10-fold, at least 50-fold, at least 100-fold, or more. “Modulating” can also involve effecting a change (which can either be an increase or a decrease) in affinity, avidity, specificity and/or selectivity of a target or antigen. “Modulating” can also mean effecting a change with respect to one or more biological or physiological mechanisms, effects, responses, functions, pathways or activities in which the target or antigen (or in which its substrate(s), ligand(s) or pathway(s) are involved, such as its signaling pathway or metabolic pathway and their associated biological or physiological effects) is involved. Again, as will be clear to the skilled person, such an action as an agonist or an antagonist can be determined in any suitable manner and/or using any suitable assay known or described herein (e.g., in vitro or cellular assay), depending on the target or antigen involved.

Modulating can, for example, also involve allosteric modulation of the target and/or reducing or inhibiting the binding of the target to one of its substrates or ligands and/or competing with a natural ligand, substrate for binding to the target. Modulating can also involve activating the target or the mechanism or pathway in which it is involved. Modulating can for example also involve effecting a change in respect of the folding or conformation of the target, or in respect of the ability of the target to fold, to change its conformation (for example, upon binding of a ligand), to associate with other (sub)units, or to disassociate. Modulating can for example also involve effecting a change in the ability of the target to signal, phosphorylate, dephosphorylate, and the like.

As used herein, an “agent” can refer to a protein-binding agent that permits modulation of activity of proteins or disrupts interactions of proteins and other biomolecules, such as but not limited to disrupting protein-protein interaction, ligand-receptor interaction, or protein-nucleic acid interaction. Agents can also refer to DNA targeting or RNA targeting agents. Agents can also refer to a protein, such as CLEC-2. Agents may include a fragment, derivative and analog of an active agent. The terms “fragment,” “derivative” and “analog” when referring to polypeptides as used herein refers to polypeptides which either retain substantially the same biological function or activity as such polypeptides. An analog includes a proprotein which can be activated by cleavage of the proprotein portion to produce an active mature polypeptide. Such agents include, but are not limited to, antibodies (“antibodies” includes antigen-binding portions of antibodies such as epitope- or antigen-binding peptides, paratopes, functional CDRs; recombinant antibodies; chimeric antibodies; humanized antibodies; nanobodies; tribodies; midibodies; or antigen-binding derivatives, analogs, variants, portions, or fragments thereof), protein-binding agents, nucleic acid molecules, small molecules, recombinant protein, peptides, aptamers, avimers and protein-binding derivatives, portions or fragments thereof. An “agent” as used herein may also refer to an agent that inhibits expression of a gene, such as but not limited to a DNA targeting agent (e.g., CRISPR system, TALE, Zinc finger protein) or RNA targeting agent (e.g., inhibitory nucleic acid molecules such as RNAi, miRNA, ribozyme).

In certain embodiments, the agent modulates CLEC-2 signaling. In certain embodiments, the agent is an agonist or antagonist of PDPN or CLEC-2 activity.

The agents of the present invention may be modified, such that they acquire advantageous properties for therapeutic use (e.g., stability and specificity), but maintain their biological activity.

It is well known that the properties of certain proteins can be modulated by attachment of polyethylene glycol (PEG) polymers, which increases the hydrodynamic volume of the protein and thereby slows its clearance by kidney filtration. (See, e.g., Clark et al., J. Biol. Chem. 271: 21969-21977 (1996)). Therefore, it is envisioned that certain agents can be PEGylated (e.g., on peptide residues) to provide enhanced therapeutic benefits such as, for example, increased efficacy by extending half-life in vivo. In certain embodiments, PEGylation of the agents may be used to extend the serum half-life of the agents and allow for particular agents to be capable of crossing the blood-brain barrier. Thus, in one embodiment, PEGylating CLEC-2, PDPN or signature gene agonists or antagonists improve the pharmacokinetics and pharmacodynamics of the agonists or antagonists.

In regards to peptide PEGylation methods, reference is made to Lu et al., Int. J. Pept. Protein Res. 43: 127-38 (1994); Lu et al., Pept. Res. 6: 140-6 (1993); Felix et al., Int. J. Pept. Protein Res. 46: 253-64 (1995); Gaertner et al., Bioconjug. Chem. 7: 38-44 (1996); Tsutsumi et al., Thromb. Haemost. 77: 168-73 (1997); Francis et al., hit. J. Hematol. 68: 1-18 (1998); Roberts et al., J. Pharm. Sci. 87: 1440-45 (1998); and Tan et al., Protein Expr. Purif. 12: 45-52 (1998). Polyethylene glycol or PEG is meant to encompass any of the forms of PEG that have been used to derivatize other proteins, including, but not limited to, mono-(C1-10) alkoxy or aryloxy-polyethylene glycol. Suitable PEG moieties include, for example, 40 kDa methoxy poly(ethylene glycol) propionaldehyde (Dow, Midland, Mich.); 60 kDa methoxy poly(ethylene glycol) propionaldehyde (Dow, Midland, Mich.); 40 kDa methoxy poly(ethylene glycol) maleimido-propionamide (Dow, Midland, Mich.); 31 kDa alpha-methyl-w-(3-oxopropoxy), polyoxyethylene (NOF Corporation, Tokyo); mPEG2-NHS-40k (Nektar); mPEG2-MAL-40k (Nektar), SUNBRIGHT GL2-400MA ((PEG)240 kDa) (NOF Corporation, Tokyo), SUNBRIGHT ME-200MA (PEG20 kDa) (NOF Corporation, Tokyo). The PEG groups are generally attached to the peptide via acylation or alkylation through a reactive group on the PEG moiety (for example, a maleimide, an aldehyde, amino, thiol, or ester group) to a reactive group on the peptide (for example, an aldehyde, amino, thiol, a maleimide, or ester group).

The PEG molecule(s) may be covalently attached to any Lys, Cys, or K(CO(CH2)2SH) residues at any position in a peptide. In certain embodiments, the antagonists or agonists described herein can be PEGylated directly to any amino acid at the N-terminus by way of the N-terminal amino group. A “linker arm” may be added to a peptide to facilitate PEGylation. PEGylation at the thiol side-chain of cysteine has been widely reported (see, e.g., Caliceti & Veronese, Adv. Drug Deliv. Rev. 55: 1261-77 (2003)). If there is no cysteine residue in the peptide, a cysteine residue can be introduced through substitution or by adding a cysteine to the N-terminal amino acid. In certain embodiments, antagonists or agonists are PEGylated through the side chains of a cysteine residue added to the N-terminal amino acid.

In exemplary embodiments, the PEG molecule(s) may be covalently attached to an amide group in the C-terminus of a peptide. In preferred embodiments, there is at least one PEG molecule covalently attached to the antagonists or agonist. In certain embodiments, the PEG molecule used in modifying an agent of the present invention is branched while in other embodiments, the PEG molecule may be linear. In particular aspects, the PEG molecule is between 1 kDa and 100 kDa in molecular weight. In further aspects, the PEG molecule is selected from 10, 20, 30, 40, 50, 60, and 80 kDa. In further still aspects, it is selected from 20, 40, or 60 kDa. Where there are two PEG molecules covalently attached to the agent of the present invention, each is 1 to 40 kDa and in particular aspects, they have molecular weights of 20 and 20 kDa, 10 and 30 kDa, 30 and 30 kDa, 20 and 40 kDa, or 40 and 40 kDa. In particular aspects, the agent (e.g., neuromedin U receptor agonists or antagonists) contain mPEG-cysteine. The mPEG in mPEG-cysteine can have various molecular weights. The range of the molecular weight is preferably 5 kDa to 200 kDa, more preferably 5 kDa to 100 kDa, and further preferably 20 kDa to 60 kDA. The mPEG can be linear or branched.

In particular embodiments, the agents (e.g., agonist or antagonists) include a protecting group covalently joined to the N-terminal amino group. In exemplary embodiments, a protecting group covalently joined to the N-terminal amino group of the antagonists or agonists reduces the reactivity of the amino terminus under in vivo conditions. Amino protecting groups include —C1-10 alkyl, —C1-10 substituted alkyl, —C2-10 alkenyl, —C2-10 substituted alkenyl, aryl, —C1-6 alkyl aryl, —C(O)—(CH2)1-6-COOH, —C(O)—C1-6 alkyl, —C(O)-aryl, —C(O)—O—C1-6 alkyl, or —C(O)—O-aryl. In particular embodiments, the amino terminus protecting group is selected from the group consisting of acetyl, propyl, succinyl, benzyl, benzyloxycarbonyl, and t-butyloxycarbonyl. In other embodiments, deamination of the N-terminal amino acid is another modification that may be used for reducing the reactivity of the amino terminus under in vivo conditions.

Chemically modified compositions of the agents (e.g., agonists or antagonists) wherein the agent is linked to a polymer are also included within the scope of the present invention. The polymer selected is usually modified to have a single reactive group, such as an active ester for acylation or an aldehyde for alkylation, so that the degree of polymerization may be controlled. Included within the scope of polymers is a mixture of polymers. Preferably, for therapeutic use of the end-product preparation, the polymer will be pharmaceutically acceptable. The polymer or mixture thereof may include, but is not limited to, polyethylene glycol (PEG), monomethoxy-polyethylene glycol, dextran, cellulose, or other carbohydrate based polymers, poly-(N-vinyl pyrrolidone) polyethylene glycol, propylene glycol homopolymers, a polypropylene oxide/ethylene oxide co-polymer, polyoxyethylated polyols (for example, glycerol), and polyvinyl alcohol.

In other embodiments, the agents are modified by PEGylation, cholesterylation, or palmitoylation. The modification can be to any amino acid residue. In preferred embodiments, the modification is to the N-terminal amino acid of the agent (e.g., agonist or antagonists), either directly to the N-terminal amino acid or by way coupling to the thiol group of a cysteine residue added to the N-terminus or a linker added to the N-terminus such as trimesoyl tris(3,5-dibromosalicylate (Ttds). In certain embodiments, the N-terminus of the agent (e.g., agonist or antagonist) comprises a cysteine residue to which a protecting group is coupled to the N-terminal amino group of the cysteine residue and the cysteine thiolate group is derivatized with N-ethylmaleimide, PEG group, cholesterol group, or palmitoyl group. In other embodiments, an acetylated cysteine residue is added to the N-terminus of the agents, and the thiol group of the cysteine is derivatized with N-ethylmaleimide, PEG group, cholesterol group, or palmitoyl group. In certain embodiments, the agent of the present invention is a conjugate. In certain embodiments, the agent of the present invention is a polypeptide consisting of an amino acid sequence which is bound with a methoxypolyethylene glycol(s) via a linker.

Substitutions of amino acids may be used to modify an agent of the present invention. The phrase “substitution of amino acids” as used herein encompasses substitution of amino acids that are the result of both conservative and non-conservative substitutions. Conservative substitutions are the replacement of an amino acid residue by another similar residue in a polypeptide. Typical but not limiting conservative substitutions are the replacements, for one another, among the aliphatic amino acids Ala, Val, Leu and Ile; interchange of Ser and Thr containing hydroxy residues, interchange of the acidic residues Asp and Glu, interchange between the amide-containing residues Asn and Gln, interchange of the basic residues Lys and Arg, interchange of the aromatic residues Phe and Tyr, and interchange of the small-sized amino acids Ala, Ser, Thr, Met, and Gly. Non-conservative substitutions are the replacement, in a polypeptide, of an amino acid residue by another residue which is not biologically similar. For example, the replacement of an amino acid residue with another residue that has a substantially different charge, a substantially different hydrophobicity, or a substantially different spatial configuration.

In certain embodiments, the present invention provides for one or more therapeutic agents. In certain embodiments, the one or more agents comprises a small molecule inhibitor, small molecule degrader (e.g., PROTAC), genetic modifying agent, antibody, antibody fragment, antibody-like protein scaffold, aptamer, protein, or any combination thereof.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein, “treating” includes ameliorating, curing, preventing it from becoming worse, slowing the rate of progression, or preventing the disorder from re-occurring (i.e., to prevent a relapse). In certain embodiments, the present invention provides for one or more therapeutic agents against combinations of targets identified. Targeting the identified combinations may provide for enhanced or otherwise previously unknown activity in the treatment of disease.

### Small Molecules

In certain embodiments, the one or more agents is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking a binding site or activating a receptor by binding to a ligand binding site).

One type of small molecule applicable to the present invention is a degrader molecule. Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810).

### Antibodies

The term “antibody” (e.g., anti-PDPN or anti-CLEC-2 antibody) is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, lgM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, γ1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by p pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains. The “variable” domains of an antibody light chain are referred to interchangeably as “light chain variable regions”, “light chain variable domains”, “VL” regions or “VL” domains. The “variable” domains of an antibody heavy chain are referred to interchangeably as “heavy chain constant regions”, “heavy chain constant domains”, “VH” regions or “VH” domains.

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention, and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain a minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the C_{H}1 domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. For example, an antagonist antibody may bind CLEC-2 receptor or PDPN and inhibit the ability to suppress a type 2 inflammatory response. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., International Patent Publication No. WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

### Nucleic Acid Agents

The disclosure also encompasses nucleic acid molecules, in particular those that inhibit a target gene. Exemplary nucleic acid molecules include aptamers, siRNA, artificial microRNA, interfering RNA or RNAi, dsRNA, ribozymes, antisense oligonucleotides, and DNA expression cassettes encoding said nucleic acid molecules. Preferably, the nucleic acid molecule is an antisense oligonucleotide. Antisense oligonucleotides (ASO) generally inhibit their target by binding target mRNA and sterically blocking expression by obstructing the ribosome. ASOs can also inhibit their target by binding target mRNA thus forming a DNA-RNA hybrid that can be a substance for RNase H. Preferred ASOs include Locked Nucleic Acid (LNA), Peptide Nucleic Acid (PNA), and morpholinos Preferably, the nucleic acid molecule is an RNAi molecule, i.e., RNA interference molecule. Preferred RNAi molecules include siRNA, shRNA, and artificial miRNA. The design and production of siRNA molecules is well known to one of skill in the art (e.g., Hajeri P B, Singh S K. Drug Discov Today. 2009 14(17-18):851-8). The nucleic acid molecule inhibitors may be chemically synthesized and provided directly to cells of interest. The nucleic acid compound may be provided to a cell as part of a gene delivery vehicle. Such a vehicle is preferably a liposome or a viral gene delivery vehicle. In certain embodiments, the agent is an agent that overexpresses a gene target described herein (e.g., a vector, such as retroviral vector).

### Genetic Modifying Agents

In certain embodiments, the one or more modulating agents may be a genetic modifying agent. The genetic modifying agent may comprise a CRISPR system, a zinc finger nuclease system, a TALEN, a meganuclease or RNAi system. In some embodiments, a polynucleotide of the present invention described elsewhere herein can be modified using a genetic modifying agent (e.g., one or more genes are selected from Table 2 or the group consisting of Mthfd2, Itga4, L1cam, 9130019O22Rik, Gal, Clec1b, Mgst3, Tgfb2, Serpina3f, Ifi47, Vcam1, Gbp6, Slamf8, Best1, H2-Q9, Ccr5, Rgs12, H2-DMb1, Tgtp2, Gbp4, Pla2g7, Plxnb2, Cd5l, Enpp5, Ifitm3, Pbx1, Pla2g2d, Tgtp1, AA467197, Ptpro, H2-Q4, Irf5, C3, Mt2, Sqle, Ch25h, Il1a, Rilpl2, Igf1, Cd52, Sc4 mol, Smpdl3b, Fbp1, Pilrb2, C1qa, H2-M2, Cdh1, Cyp1a1, Ccl9, Marco, Plxna1, Cxcl16, Trpm2, Ccdc80 and Cd300a; or one or more genes are selected from the group consisting of Plxnb2, Sqle, H2-Q9, Tgtp1, Cdh1, H2-M2, Ccdc80, C3, Cd300a, Slamf8, Mt2, Serpina3f, Cd52, Rilpl2, Enpp5, Rgs12, Pla2g7, Gbp4, Irf5, Best1, H2-Q4, Ccl9, Ccr5, Igf1, Cxcl16, C1qa, Trpm2, Vcam1, Fbp1, Ch25h, Marco, AA467197, Pla2g2d, Smpdl3b, H2-DMb1, Gbp6, Tgtp2, Ifi47, Il1a, Sc4 mol, Cyp1a1, Pilrb2, Plxna1, Ifitm3, Pbx1, Cd5l, Ptpro, Ctla4, Ptgs1 and Mmab).

### CRISPR-Cas Modification

In some embodiments, a polynucleotide of the present invention described elsewhere herein can be modified using a CRISPR-Cas and/or Cas-based system.

In general, a CRISPR-Cas or CRISPR system as used in herein and in documents, such as International Patent Publication No. WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two class are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

### Class 1 CRISPR-Cas Systems

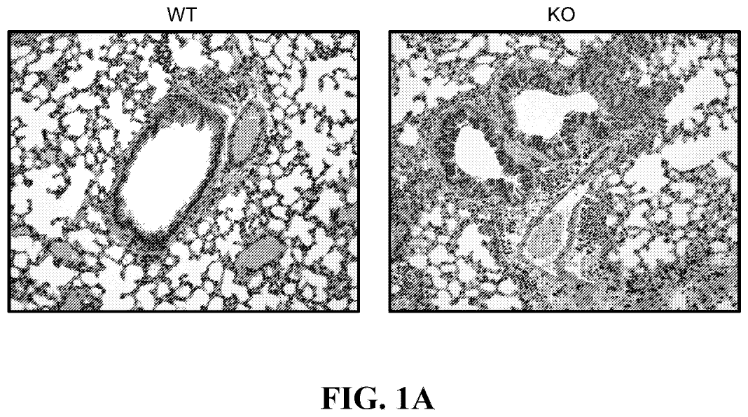

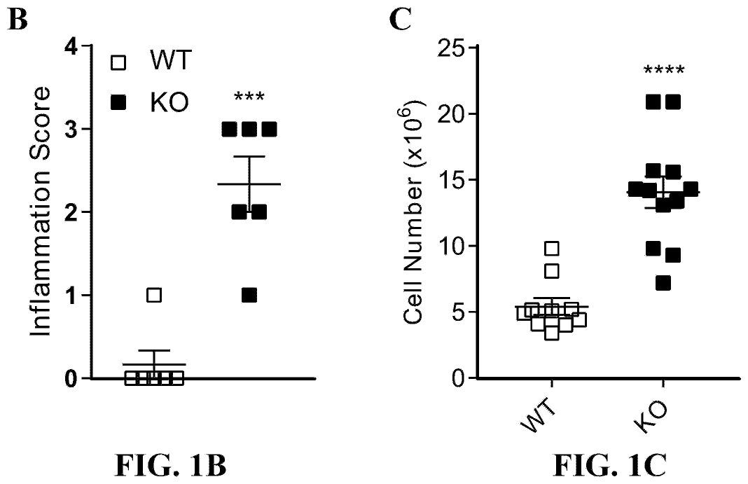

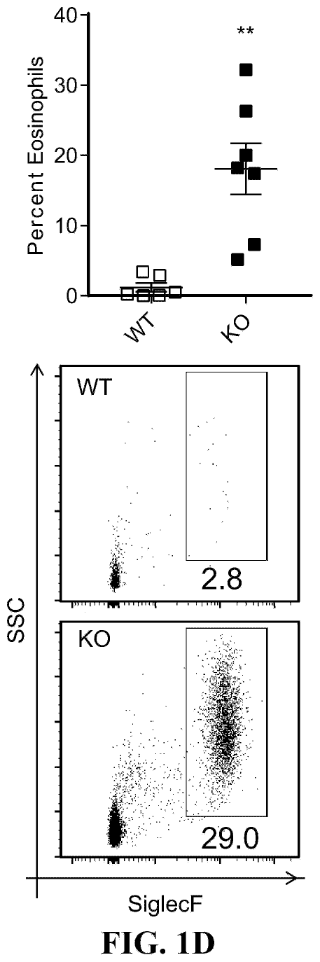

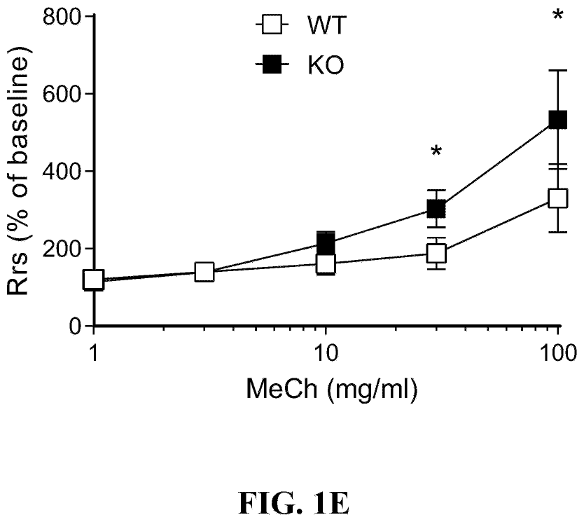

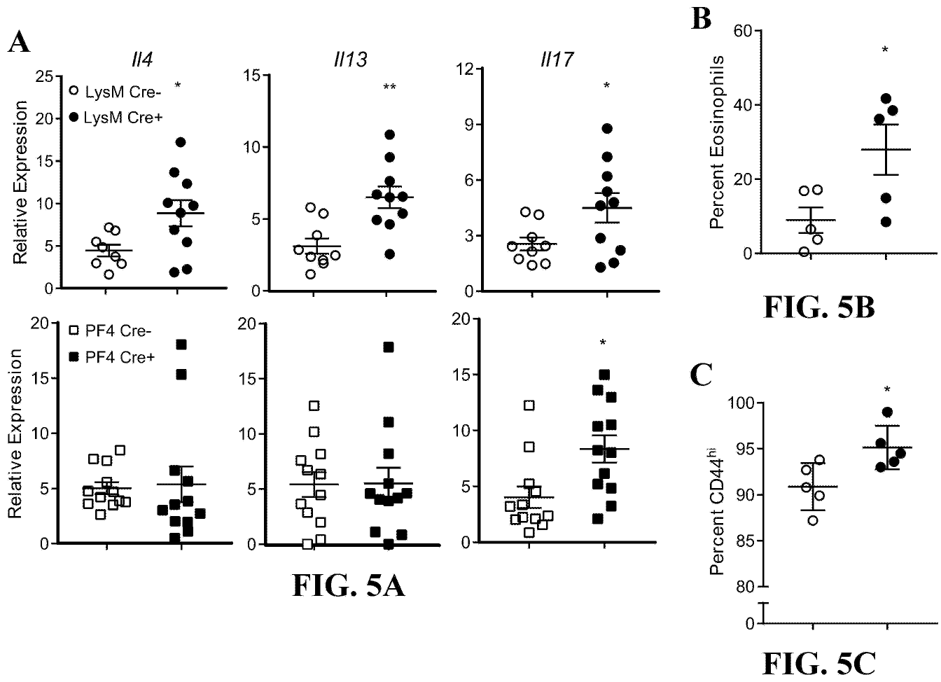

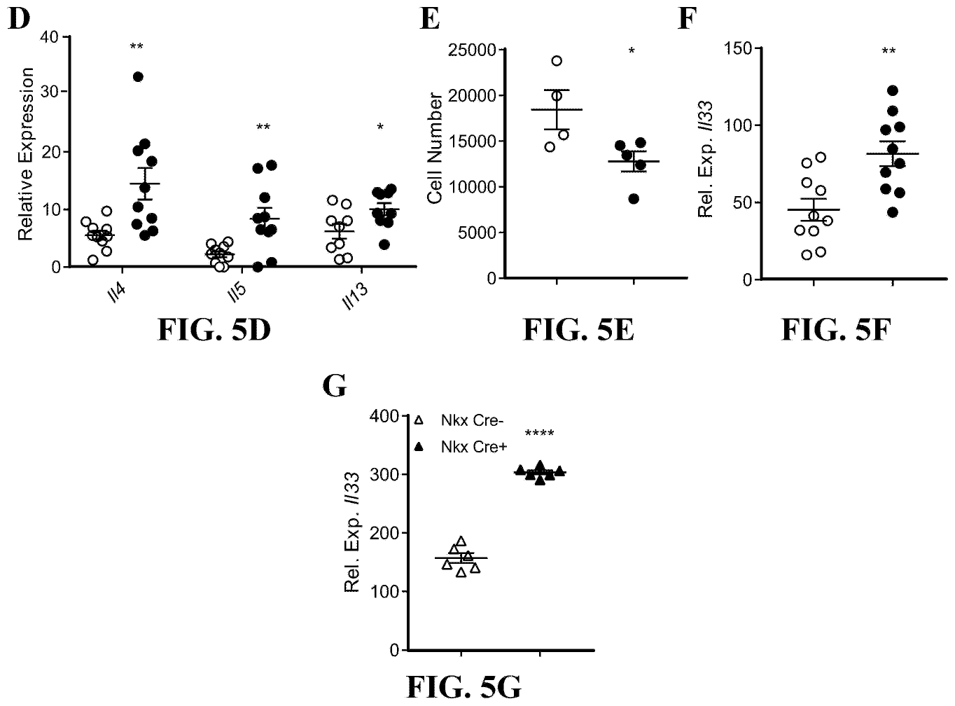

In some embodiments, the CRISPR-Cas system that can be used to modify a polynucleotide of the present invention described herein can be a Class 1 CRISPR-Cas system. Class 1 CRISPR-Cas systems are divided into types I, II, and IV. Makarova et al. 2020. Nat. Rev. 18: 67-83., particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-C, III-D, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n5, FIG. 5.

The Class 1 systems typically comprise a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

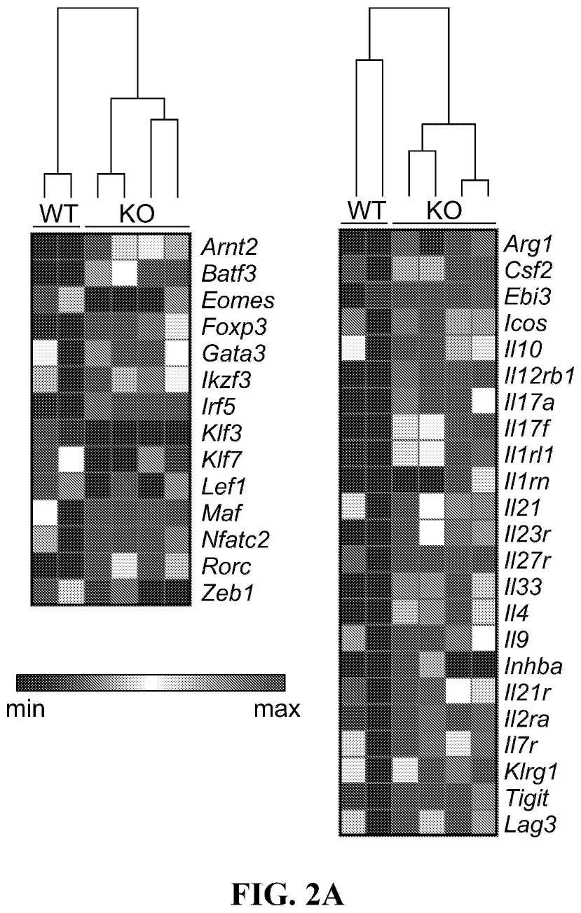

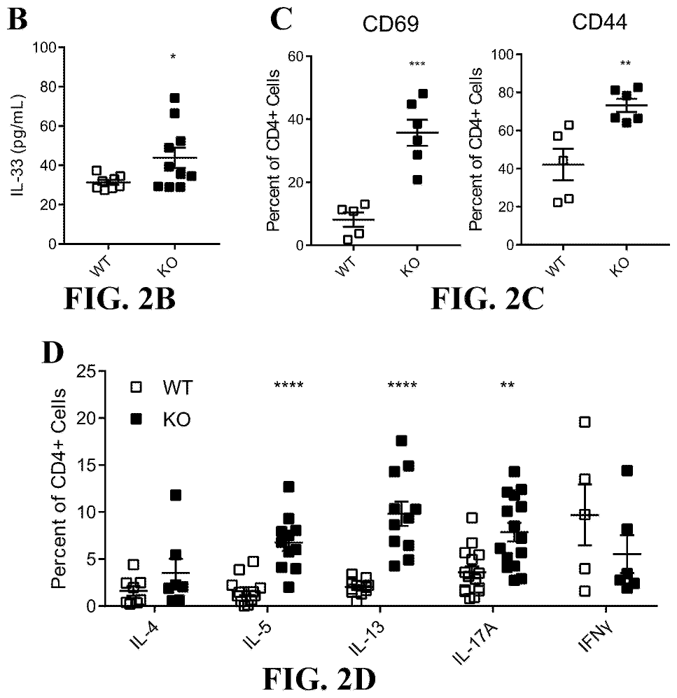

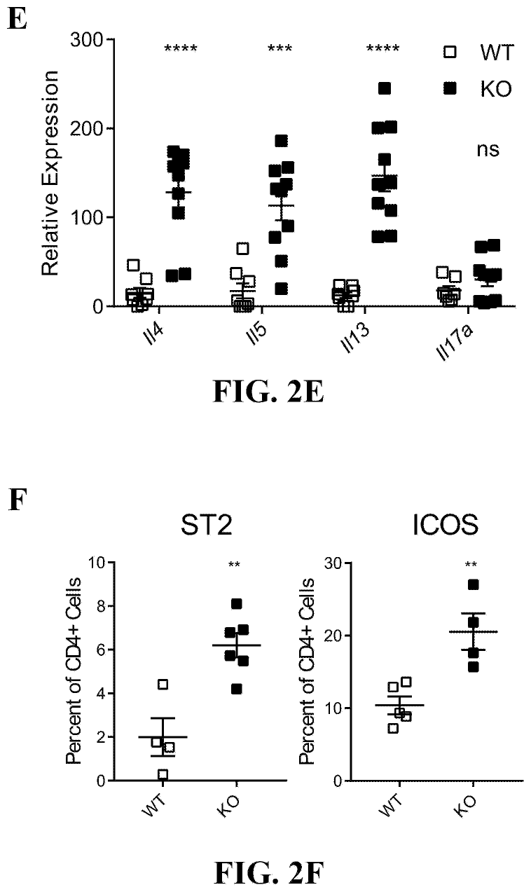

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cas11). See, e.g., FIGS. 1 and 2. Koonin E V, Makarova K S. 2019 Origins and Evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cas6, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), Cas12d (CasY), Cas12e (CasX), and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (International Patent Publication Nos. WO 2019/005884 and WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and International Patent Publication WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C⋅G base pair into a T⋅A base pair (Komor et al. 2016. Nature. 533:420-424; Nishida et al. 2016. Science. 353; and Li et al. Nat. Biotech. 36:324-327) and ABEs convert an A⋅T base pair to a G⋅C base pair. Collectively, CBEs and ABEs can mediate all four possible transition mutations (C to T, A to G, T to C, and G to A). Rees and Liu. 2018.Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

In certain example embodiments, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358: 1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstituble halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system. See e.g. Anzalone et al. 2019. Nature. 576: 149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion and combinations thereof. Generally, a prime editing system, as exemplified by PE1, PE2, and PE3 (Id), can include a reverse transcriptase fused or otherwise coupled or associated with an RNA-programmable nickase and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g. a prime editing guide molecule or peg guide molecule) directly into the target site in the target polynucleotide. See e.g. Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g. is a Cas9 nickase). In some embodiments, the Cas polypeptide is fused to the reverse transcriptase. In some embodiments, the Cas polypeptide is linked to the reverse transcriptase.

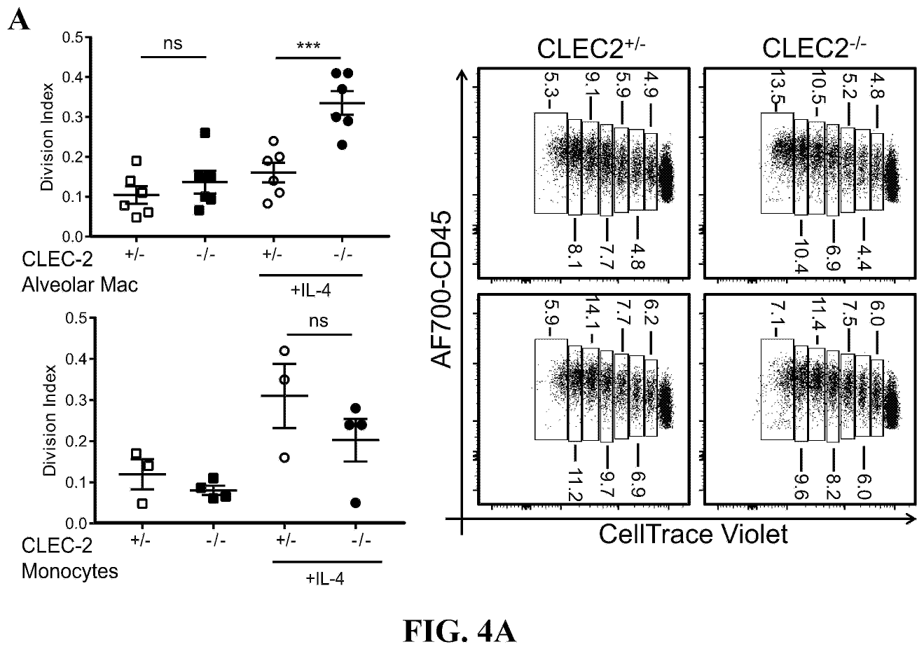

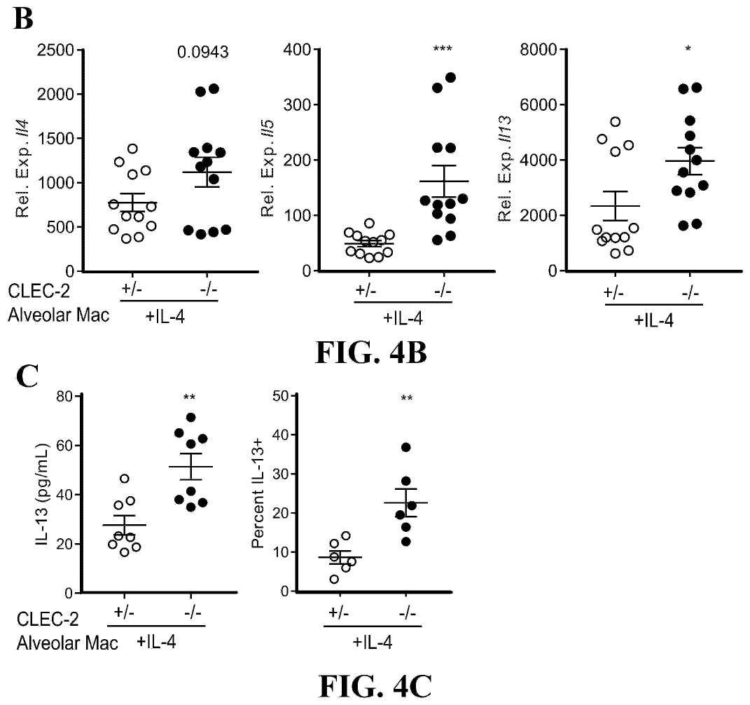

In some embodiments, the prime editing system can be a PE1 system or variant thereof, a PE2 system or variant thereof, or a PE3 (e.g. PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 3 (from Gleditzsch et al. 2019) below shows several Cas polypeptides and the PAM sequence they recognize.

TABLE 3
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including LbCpf1 | TTTV |
| and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein His A, CorU.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Zinc Finger Nucleases

In some embodiments, the polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Sequences Related to Nucleus Targeting and Transportation

In some embodiments, one or more components (e.g., the Cas protein and/or deaminase) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO:1) or PKKKRKVEAS (SEQ ID NO:2); the NLS from nucleoplasmin (e.g., the nucleoplasmin bipartite NLS with the sequence KRPAATKKAGQAKKKK (SEQ ID NO:3)); the c-myc NLS having the amino acid sequence PAAKRVKLD (SEQ ID NO:4) or RQRRNELKRSP (SEQ ID NO:5); the hRNPA1 M9 NLS having the sequence NQSSNFGPMKGGNFGGRSSGPYGGGGQYFAKPRNQGGY (SEQ ID NO:6); the sequence RMRIZFKNKGKDTAELRRRRVEVSVELRKAKKDEQILKRRNV (SEQ ID NO:7) of the IBB domain from importin-alpha; the sequences VSRKRPRP (SEQ ID NO:8) and PPKKARED (SEQ ID NO:9) of the myoma T protein; the sequence PQPKKKPL (SEQ ID NO:10) of human p53; the sequence SALIKKKKKMAP (SEQ ID NO:11) of mouse c-abl IV; the sequences DRLRR (SEQ ID NO:12) and PKQKKRK (SEQ ID NO:13) of the influenza virus NS1; the sequence RKLKKKIKKL (SEQ ID NO:14) of the Hepatitis virus delta antigen; the sequence REKKKFLKRR (SEQ ID NO:15) of the mouse Mxl protein; the sequence KRKGDEVDGVDEVAKKKSKK (SEQ ID NO:16) of the human poly(ADP-ribose) polymerase; and the sequence RKCLQAGMNLEARKTKK (SEQ ID NO:17) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g. aptamers) for adapter proteins, which may be linked to or fused to a nucleotide deaminase or catalytic domain thereof. When such a guide forms a CRISPR complex (e.g., CRISPR-Cas protein binding to guide and target), the adapter proteins bind and the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g. due to steric hindrance within the three-dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Templates

In some embodiments, the composition for engineering cells comprise a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include a sequence that corresponds to both, a first site on the target sequence that is cleaved in a first Cas protein mediated event, and a second site on the target sequence that is cleaved in a second Cas protein mediated event.

In certain embodiments, the template nucleic acid can include a sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include a sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include a sequence which, when integrated, results in decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include a sequence which results in a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or 34 or 35, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or ₃₄ or ₃₅)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

M D P I R S R T P S P A R E L L S G P Q P D G V Q
P T A D R G V S P P A G G P L D G L P A R R T M S
R T R L P S P P A P S P A F S A D S F S D L L R Q
F D P S L F N T S L F D S L P P F G A H H T E A A
T G E W D E V Q S G L R A A D A P P P T M R V A V
T A A R P P R A K P A P R R R A A Q P S D A S P A
A Q V D L R T L G Y S Q Q Q Q E K I K P K V R S T
V A Q H H E A L V G H G F T H A H I V A L S Q H P
A A L G T V A V K Y Q D

An exemplary amino acid sequence of a C-terminal capping region is:

(SEQ ID NO: 19)
R P A L E S I V A Q L S R P D P A L A A L T N D H
L V A L A C L G G R P A L D A V K K G L P H A P A
L I K R T N R R I P E R T S H R V A D H A Q V V R
V L G F F Q C H S H P A Q A F D D A M T Q F G M S
R H G L L Q L F R R V G V T E L E A R S G T L P P
A S Q R W D R I L Q A S G M K R A K P S P T S T Q
T P D Q A S L H A F A D S L E R D L D A P S P M H
E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### RNAi

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated herein by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Standard Anti-Inflammatory Treatment

In certain embodiments, a standard anti-inflammatory treatment is administered to treat a type 2 or 3 immune response. In certain embodiments, the standard treatment can include long-term treatment. Non-limiting types of long-term control medications include: oral and inhaled corticosteroids. These anti-inflammatory drugs include prednisone, methylprednisolone, fluticasone (Flonase, Flovent HFA), budesonide (Pulmicort Flexhaler, Rhinocort), flunisolide (Aerospan HFA), ciclesonide (Alvesco, Omnaris, Zetonna), beclomethasone (Qnasl, Qvar), mometasone (Asmanex) and fluticasone furoate (Arnuity Ellipta). Anti-inflammatory drugs also include leukotriene modifiers. These oral medications include montelukast (Singulair), zafirlukast (Accolate) and zileuton (Zyflo). Anti-inflammatory drugs also include long-acting beta agonists. These inhaled medications, which include salmeterol (Serevent) and formoterol (Foradil, Perforomist), open the airways. Some research shows that they may increase the risk of a severe asthma attack, so they are taken in combination with an inhaled corticosteroid. Anti-inflammatory drugs also include combination inhalers. These medications, such as fluticasone-salmeterol (Advair Diskus), budesonide-formoterol (Symbicort) and formoterol-mometasone (Dulera), contain a long-acting beta agonist along with a corticosteroid. Anti-inflammatory drugs also include theophylline. Theophylline (Theo-24, Elixophyllin, others) is a daily pill that helps keep the airways open (bronchodilator) by relaxing the muscles around the airways. In certain embodiments, treatments targeting different alarmins are used (3,4).

### Diseases

It will be understood by the skilled person that treating as referred to herein encompasses enhancing treatment or improving treatment efficacy. Treatment may include inhibition of an inflammatory response, enhancing an immune response, tumor regression as well as inhibition of tumor growth, metastasis or tumor cell proliferation, or inhibition or reduction of otherwise deleterious effects associated with the tumor.

Efficaciousness of treatment is determined in association with any known method for diagnosing or treating the particular disease. The invention comprehends a treatment method comprising any one of the methods or uses herein discussed.

The phrase “therapeutically effective amount” as used herein refers to a sufficient amount of a drug, agent, or compound to provide a desired therapeutic effect.

As used herein “patient” refers to any human being receiving or who may receive medical treatment and is used interchangeably herein with the term “subject”.

Therapy or treatment according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the cancer, and how the patient responds to the treatment. Additionally, a person having a greater risk of developing an inflammatory response (e.g., a person who is genetically predisposed or predisposed to allergies or a person having a disease characterized by episodes of inflammation) may receive prophylactic treatment to inhibit or delay symptoms of the disease.

A skilled person can readily determine diseases that can be treated by reducing a type 2 inflammatory response. Type 2 inflammatory responses have been associated with allergic asthma, therapy resistant-asthma, steroid-resistant severe allergic airway inflammation, systemic steroid-dependent severe eosinophilic asthma, chronic rhino-sinusitis (CRS), atopic dermatitis, food allergies, persistence of chronic airway inflammation, and primary eosinophilic gastrointestinal disorders (EGIDs), including but not limited to eosinophilic esophagitis (EoE), eosinophilic gastritis, eosinophilic gastroenteritis, and eosinophilic colitis (see, e.g., Van Rijt et al., Type 2 innate lymphoid cells: at the cross-roads in allergic asthma, Seminars in Immunopathology July 2016, Volume 38, Issue 4, pp 483-496; Rivas et al., IL-4 production by group 2 innate lymphoid cells promotes food allergy by blocking regulatory T-cell function, J Allergy Clin Immunol. 2016 September; 138(3):801-811.e9; and Morita, Hideaki et al. Innate lymphoid cells in allergic and nonallergic inflammation, Journal of Allergy and Clinical Immunology, Volume 138, Issue 5, 1253-1264). Asthma is characterized by recurrent episodes of wheezing, shortness of breath, chest tightness, and coughing. Sputum may be produced from the lung by coughing but is often hard to bring up. During recovery from an attack, it may appear pus-like due to high levels of eosinophils. Symptoms are usually worse at night and in the early morning or in response to exercise or cold air. Some people with asthma rarely experience symptoms, usually in response to triggers, whereas others may have marked and persistent symptoms. CRS is characterized by inflammation of the mucosal surfaces of the nose and para-nasal sinuses, and it often coexists with allergic asthma. Atopic dermatitis is a chronic inflammatory skin disease that is characterized by eosinophilic infiltration and high serum IgE levels. Similar to allergic asthma and CRS, atopic dermatitis has been associated with increased expression of TSLP, IL-25, and IL-33 in the skin. Primary eosinophilic gastrointestinal disorders (EGIDs), including eosinophilic esophagitis (EoE), eosinophilic gastritis, eosinophilic gastroenteritis, and eosinophilic colitis, are disorders that exhibit eosinophil-rich inflammation in the gastrointestinal tract in the absence of known causes for eosinophilia such as parasite infection and drug reaction. Applicants have discovered factors that balance homeostatic and pathological pro-inflammatory type 2 responses. In certain embodiments, modulation of these factors, as described herein, may be used to treat the diseases described. In preferred embodiments, CLEC-2 signaling is modulated. In certain embodiments, the treatment can maintain homeostasis of macrophages.

In certain embodiments, a type 2 mediated disease or disorder that can be treated by reducing a type 2 inflammatory response or maintaining homeostasis may be any inflammatory disease or disorder such as, but not limited to, asthma, allergy, allergic rhinitis, allergic airway inflammation, atopic dermatitis (AD), chronic obstructive pulmonary disease (COPD), inflammatory bowel disease (IBD), multiple sclerosis, arthritis, psoriasis, eosinophilic esophagitis, eosinophilic pneumonia, eosinophilic psoriasis, hypereosinophilic syndrome, graft-versus-host disease, uveitis, cardiovascular disease, pain, multiple sclerosis, lupus, vasculitis, chronic idiopathic urticaria and Eosinophilic Granulomatosis with Polyangiitis (Churg-Strauss Syndrome).

The asthma may be allergic asthma, non-allergic asthma, severe refractory asthma, asthma exacerbations, viral-induced asthma or viral-induced asthma exacerbations, steroid resistant asthma, steroid sensitive asthma, eosinophilic asthma or non-eosinophilic asthma and other related disorders characterized by airway inflammation or airway hyperresponsiveness (AHR).

The COPD may be a disease or disorder associated in part with, or caused by, cigarette smoke, air pollution, occupational chemicals, allergy or airway hyperresponsiveness.

The allergy may be associated with foods, pollen, mold, dust mites, animals, or animal dander.

The IBD may be ulcerative colitis (UC), Crohn's Disease, collagenous colitis, lymphocytic colitis, ischemic colitis, diversion colitis, Behcet's syndrome, infective colitis, indeterminate colitis, and other disorders characterized by inflammation of the mucosal layer of the large intestine or colon.

The arthritis may be selected from the group consisting of osteoarthritis, rheumatoid arthritis and psoriatic arthritis.

The disclosure also provides methods for enhancing a type 2 response and treating disease. In certain embodiments, macrophages are switched to inflammatory macrophages. In certain embodiments, induction of inflammatory macrophages (e.g., signature in CLEC-2 knockout cells) may be used in treating cancer. In one embodiment, modulation of CLEC-2 signaling is used for inducing an inflammatory immune response state for the treatment of cancer.

The cancer may include, without limitation, liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or multiple myeloma.

The cancer may include, without limitation, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumour including serous tumour, endometrioid tumor and mucinous cystadenocarcinoma, sex-cord-stromal tumor), prostate cancer, liver and bile duct carcinoma (e.g., hepatocelluar carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, medullablastoma, astrocytoma, medulloblastoma, craniopharyngioma, ependymoma, pinealoma, hemangioblastoma, acoustic neuroma, oligodenroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

### Administration

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The medicaments of the invention are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the invention may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

The agents disclosed herein may be used in a pharmaceutical composition when combined with a pharmaceutically acceptable carrier. Such compositions comprise a therapeutically-effective amount of the agent and a pharmaceutically acceptable carrier. Such a composition may also further comprise (in addition to an agent and a carrier) diluents, fillers, salts, buffers, stabilizers, solubilizers, and other materials well known in the art. Compositions comprising the agent can be administered in the form of salts provided the salts are pharmaceutically acceptable. Salts may be prepared using standard procedures known to those skilled in the art of synthetic organic chemistry.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to specific agents (e.g., neuromedin U receptor agonists or antagonists), also include the pharmaceutically acceptable salts thereof.

Methods of administrating the pharmacological compositions, including agonists, antagonists, antibodies or fragments thereof, to an individual include, but are not limited to, intradermal, intrathecal, intramuscular, intraperitoneal, intravenous, subcutaneous, intranasal, epidural, by inhalation, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Various delivery systems are known and can be used to administer the pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (e.g., a tumor), thus requiring only a fraction of the systemic dose. See, for example, Goodson, In: Medical Applications of Controlled Release, 1984. (CRC Press, Boca Raton, Fla.).

The amount of the agents which will be effective in the treatment of a particular disorder or condition will depend on the nature of the disorder or condition and may be determined by standard clinical techniques by those of skill within the art. In addition, in vitro assays may optionally be employed to help identify optimal dosage ranges. The precise dose to be employed in the formulation will also depend on the route of administration and the overall seriousness of the disease or disorder, and should be decided according to the judgment of the practitioner and each patient's circumstances. Ultimately, the attending physician will decide the amount of the agent with which to treat each individual patient. In certain embodiments, the attending physician will administer low doses of the agent and observe the patient's response. Larger doses of the agent may be administered until the optimal therapeutic effect is obtained for the patient, and at that point the dosage is not increased further. In general, the daily dose range lies within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases. In certain embodiments, suitable dosage ranges for intravenous administration of the agent are generally about 5-500 micrograms (g) of active compound per kilogram (Kg) body weight. Suitable dosage ranges for intranasal administration are generally about 0.01 pg/kg body weight to 1 mg/kg body weight. In certain embodiments, a composition containing an agent of the present invention is subcutaneously injected in adult patients with dose ranges of approximately 5 to 5000 μg/human and preferably approximately 5 to 500 μg/human as a single dose. It is desirable to administer this dosage 1 to 3 times daily. Effective doses may be extrapolated from dose-response curves derived from in vitro or animal model test systems. Suppositories generally contain active ingredient in the range of 0.5% to 10% by weight; oral formulations preferably contain 10% to 95% active ingredient. Ultimately the attending physician will decide on the appropriate duration of therapy using compositions of the present invention. Dosage will also vary according to the age, weight and response of the individual patient.

Methods for administering antibodies for therapeutic use is well known to one skilled in the art. In certain embodiments, small particle aerosols of antibodies or fragments thereof may be administered (see e.g., Piazza et al., J. Infect. Dis., Vol. 166, pp. 1422-1424, 1992; and Brown, Aerosol Science and Technology, Vol. 24, pp. 45-56, 1996). In certain embodiments, antibodies are administered in metered-dose propellant driven aerosols. In preferred embodiments, antibodies are used as agonists to depress inflammatory diseases or allergen-induced asthmatic responses. In certain embodiments, antibodies may be administered in liposomes, i.e., immunoliposomes (see, e.g., Maruyama et al., Biochim. Biophys. Acta, Vol. 1234, pp. 74-80, 1995). In certain embodiments, immunoconjugates, immunoliposomes or immunomicrospheres containing an agent of the present invention is administered by inhalation.

In certain embodiments, antibodies may be topically administered to mucosa, such as the oropharynx, nasal cavity, respiratory tract, gastrointestinal tract, eye such as the conjunctival mucosa, vagina, urogenital mucosa, or for dermal application. In certain embodiments, antibodies are administered to the nasal, bronchial or pulmonary mucosa. In order to obtain optimal delivery of the antibodies to the pulmonary cavity in particular, it may be advantageous to add a surfactant such as a phosphoglyceride, e.g. phosphatidylcholine, and/or a hydrophilic or hydrophobic complex of a positively or negatively charged excipient and a charged antibody of the opposite charge.

Other excipients suitable for pharmaceutical compositions intended for delivery of antibodies to the respiratory tract mucosa may be a) carbohydrates, e.g., monosaccharides such as fructose, galactose, glucose. D-mannose, sorbiose, and the like; disaccharides, such as lactose, trehalose, cellobiose, and the like; cyclodextrins, such as 2-hydroxypropyl-β-cyclodextrin; and polysaccharides, such as raffinose, maltodextrins, dextrans, and the like; b) amino acids, such as glycine, arginine, aspartic acid, glutamic acid, cysteine, lysine and the like; c) organic salts prepared from organic acids and bases, such as sodium citrate, sodium ascorbate, magnesium gluconate, sodium gluconate, tromethamine hydrochloride, and the like: d) peptides and proteins, such as aspartame, human serum albumin, gelatin, and the like; e) alditols, such mannitol, xylitol, and the like, and f) polycationic polymers, such as chitosan or a chitosan salt or derivative.

For dermal application, the antibodies of the present invention may suitably be formulated with one or more of the following excipients: solvents, buffering agents, preservatives, humectants, chelating agents, antioxidants, stabilizers, emulsifying agents, suspending agents, gel-forming agents, ointment bases, penetration enhancers, and skin protective agents.

Examples of solvents are e.g. water, alcohols, vegetable or marine oils (e.g. edible oils like almond oil, castor oil, cacao butter, coconut oil, corn oil, cottonseed oil, linseed oil, olive oil, palm oil, peanut oil, poppy seed oil, rapeseed oil, sesame oil, soybean oil, sunflower oil, and tea seed oil), mineral oils, fatty oils, liquid paraffin, polyethylene glycols, propylene glycols, glycerol, liquid polyalkylsiloxanes, and mixtures thereof.

Examples of buffering agents are e.g. citric acid, acetic acid, tartaric acid, lactic acid, hydrogenphosphoric acid, diethyl amine etc. Suitable examples of preservatives for use in compositions are parabenes, such as methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol, or mixtures of preservatives.

Examples of humectants are glycerin, propylene glycol, sorbitol, lactic acid, urea, and mixtures thereof.

Examples of antioxidants are butylated hydroxy anisole (BHA), ascorbic acid and derivatives thereof, tocopherol and derivatives thereof, cysteine, and mixtures thereof.

Examples of emulsifying agents are naturally occurring gums, e.g. gum acacia or gum tragacanth; naturally occurring phosphatides, e.g. soybean lecithin, sorbitan monooleate derivatives: wool fats; wool alcohols; sorbitan esters; monoglycerides; fatty alcohols; fatty acid esters (e.g. triglycerides of fatty acids); and mixtures thereof.

Examples of suspending agents are e.g. celluloses and cellulose derivatives such as, e.g., carboxymethyl cellulose, hydroxyethylcellulose, hydroxypropylcellulose, hydroxypropylmethylcellulose, carraghenan, acacia gum, arabic gum, tragacanth, and mixtures thereof.

Examples of gel bases, viscosity-increasing agents or components which are able to take up exudate from a wound are liquid paraffin, polyethylene, fatty oils, colloidal silica or aluminum, zinc soaps, glycerol, propylene glycol, tragacanth, carboxyvinyl polymers, magnesium-aluminum silicates, Carbopol®, hydrophilic polymers such as, e.g. starch or cellulose derivatives such as, e.g., carboxymethylcellulose, hydroxyethylcellulose and other cellulose derivatives, water-swellable hydrocolloids, carragenans, hyaluronates (e.g. hyaluronate gel optionally containing sodium chloride), and alginates including propylene glycol alginate.

Examples of ointment bases are e.g. beeswax, paraffin, cetanol, cetyl palmitate, vegetable oils, sorbitan esters of fatty acids (Span), polyethylene glycols, and condensation products between sorbitan esters of fatty acids and ethylene oxide, e.g. polyoxyethylene sorbitan monooleate (Tween).

Examples of hydrophobic or water-emulsifying ointment bases are paraffins, vegetable oils, animal fats, synthetic glycerides, waxes, lanolin, and liquid polyalkylsiloxanes. Examples of hydrophilic ointment bases are solid macrogols (polyethylene glycols). Other examples of ointment bases are triethanolamine soaps, sulphated fatty alcohol and polysorbates.

Examples of other excipients are polymers such as carmellose, sodium carmellose, hydroxypropylmethylcellulose, hydroxyethylcellulose, hydroxypropylcellulose, pectin, xanthan gum, locust bean gum, acacia gum, gelatin, carbomer, emulsifiers like vitamin E, glyceryl stearates, cetanyl glucoside, collagen, carrageenan, hyaluronates and alginates and chitosans.

The dose of antibody required in humans to be effective in the treatment or prevention of allergic inflammation differs with the type and severity of the allergic condition to be treated, the type of allergen, the age and condition of the patient, etc. Typical doses of antibody to be administered are in the range of 1 μg to 1 g, preferably 1 to 1000 g, more preferably 2 to 500, even more preferably 5 to 50, most preferably 10 to 20 μg per unit dosage form. In certain embodiments, infusion of antibodies of the present invention may range from 10 to 500 mg/m².

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection.

In certain embodiments, a device is used to administer the one or more agents. As used herein, an administration device can be any pharmaceutically acceptable device adapted to deliver a composition of the invention (e.g., to a subject's nose). A nasal administration device can be a metered administration device (metered volume, metered dose, or metered-weight) or a continuous (or substantially continuous) aerosol-producing device. Suitable nasal administration devices also include devices that can be adapted or modified for nasal administration. In some embodiments, the nasally administered dose can be absorbed into the bloodstream of a subject.

A metered nasal administration device delivers a fixed (metered) volume or amount (dose) of a nasal composition upon each actuation. Exemplary metered dose devices for nasal administration include, by way of example and without limitation, an atomizer, sprayer, dropper, squeeze tube, squeeze-type spray bottle, pipette, ampule, nasal cannula, metered dose device, nasal spray inhaler, breath actuated bi-directional delivery device, pump spray, pre-compression metered dose spray pump, monospray pump, bispray pump, and pressurized metered dose device. The administration device can be a single-dose disposable device, single-dose reusable device, multi-dose disposable device or multi-dose reusable device. The compositions of the invention can be used with any known metered administration device.

A continuous aerosol-producing device delivers a mist or aerosol comprising droplet of a nasal composition dispersed in a continuous gas phase (such as air). A nebulizer, pulsating aerosol nebulizer, and a nasalcontinuous positive air pressure device are exemplary of such a device. Suitable nebulizers include, by way of example and without limitation, an air driven jet nebulizer, ultrasonic nebulizer, capillary nebulizer, electromagnetic nebulizer, pulsating membrane nebulizer, pulsating plate (disc) nebulizer, pulsating/vibrating mesh nebulizer, vibrating plate nebulizer, a nebulizer comprising a vibration generator and an aqueous chamber, a nebulizer comprising a nozzle array, and nebulizers that extrude a liquid formulation through a self-contained nozzle array.

In certain embodiments, the device can be any commercially available administration devices that are used or can be adapted for nasal administration of a composition of the invention (see, e.g., US Patent Publication No. US2009-0312724A1).

In another aspect, provided is a pharmaceutical pack or kit, comprising one or more containers filled with one or more of the ingredients of the pharmaceutical compositions and agonists or antagonists.

### Diagnostic and Screening Methods

The invention provides biomarkers (e.g., phenotype specific or cell type) for the identification, diagnosis, prognosis and manipulation of cell properties, for use in a variety of diagnostic and/or therapeutic indications (e.g., for any disease described herein). Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein.

Biomarkers are useful in methods of diagnosing, prognosing and/or staging an immune response in a subject by detecting a first level of expression, activity and/or function of one or more biomarker and comparing the detected level to a control of level wherein a difference in the detected level and the control level indicates that the presence of an immune response in the subject.

The terms “diagnosis” and “monitoring” are commonplace and well-understood in medical practice. By means of further explanation and without limitation, the term “diagnosis” generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The biomarkers of the present invention are useful in methods of identifying patient populations at risk or suffering from an immune response based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments. In one aspect, the present invention provides for a method of treatment comprising: a) detecting a Type 2 and/or type 3 inflammatory state in a subject, by detecting in macrophages obtained from the subject the expression or activity of one or more genes selected from Table 2; or the group consisting of Plxnb2, Sqle, H2-Q9, Tgtp1, Cdh1, H2-M2, Ccdc80, C3, Cd300a, Slamf8, Mt2, Serpina3f, Cd52, Rilpl2, Enpp5, Rgs12, Pla2g7, Gbp4, Irf5, Best1, H2-Q4, Ccl9, Ccr5, Igf1, Cxcl16, C1qa, Trpm2, Vcam1, Fbp1, Ch25h, Marco, AA467197, Pla2g2d, Smpdl3b, H2-DMb1, Gbp6, Tgtp2, Ifi47, Il1a, Sc4 mol, Cyp1a1, Pilrb2, Plxna1, Ifitm3, Pbx1, Cd5l, Ptpro, Ctla4, Ptgs1 and Mmab; or the group consisting of Mthfd2, Itga4, L1cam, 9130019O22Rik, Gal, Clec1b, Mgst3, Tgfb2, Serpina3f, Ifi47, Vcam1, Gbp6, Slamf8, Best1, H2-Q9, Ccr5, Rgs12, H2-DMb1, Tgtp2, Gbp4, Pla2g7, Plxnb2, Cd5l, Enpp5, Ifitm3, Pbx1, Pla2g2d, Tgtp1, AA467197, Ptpro, H2-Q4, Irf5, C3, Mt2, Sqle, Ch25h, Il1a, Rilpl2, Igf1, Cd52, Sc4 mol, Smpdl3b, Fbp1, Pilrb2, C1qa, H2-M2, Cdh1, Cyp1a1, Ccl9, Marco, Plxna1, Cxcl16, Trpm2, Ccdc80 and Cd300a, wherein upregulation of upregulated genes in Table 2 and downregulation of downregulated genes in Table 2 indicate an inflammatory state; and b) treating the subject according to any embodiment herein or a standard anti-inflammatory treatment when the expression or activity of the one or more genes are detected in macrophages. The treatment may comprise administering to the subject a therapeutically effective amount of one or more agents capable of modulating CLEC-2 signaling, wherein a Type 2 inflammatory immune response is suppressed or homeostasis is maintained. For example, one or more agents that shift the signature to downregulate the upregulated genes and upregulate the down regulated genes.

The term “monitoring” generally refers to the follow-up of a disease or a condition in a subject for any changes which may occur over time.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

Suitably, an altered quantity or phenotype of the immune cells in the subject compared to a control subject having normal immune status or not having a disease comprising an immune component indicates that the subject has an impaired immune status or has a disease comprising an immune component or would benefit from an immune therapy.

Hence, the methods may rely on comparing the quantity of immune cell populations, biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterised by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises 40≥%, ≥50%, ≥60%, ≥70%, ≥75% or ≥80% or ≥85% or ≥90% or ≥95% or even≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

In one embodiment, the signature genes, biomarkers, and/or cells may be detected or isolated by immunofluorescence, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25).

In certain embodiments, diseases related to type 2 responses as described further herein are diagnosed, prognosed, or monitored. For example, a tissue sample may be obtained and analyzed for specific cell markers (IHC) or specific transcripts (e.g., RNA-FISH). Tissue samples for diagnosis, prognosis or detecting may be obtained by endoscopy. In one embodiment, a sample may be obtained by endoscopy and analyzed b FACS. As used herein, “endoscopy” refers to a procedure that uses an endoscope to examine the interior of a hollow organ or cavity of the body. The endoscope may include a camera and a light source. The endoscope may include tools for dissection or for obtaining a biological sample. A cutting tool can be attached to the end of the endoscope, and the apparatus can then be used to perform surgery. Applications of endoscopy that can be used with the present invention include, but are not limited to examination of the oesophagus, stomach and duodenum (esophagogastroduodenoscopy); small intestine (enteroscopy); large intestine/colon (colonoscopy, sigmoidoscopy); bile duct; rectum (rectoscopy) and anus (anoscopy), both also referred to as (proctoscopy); respiratory tract; nose (rhinoscopy); lower respiratory tract (bronchoscopy); ear (otoscope); urinary tract (cystoscopy); female reproductive system (gynoscopy); cervix (colposcopy); uterus (hysteroscopy); fallopian tubes (falloposcopy); normally closed body cavities (through a small incision); abdominal or pelvic cavity (laparoscopy); interior of a joint (arthroscopy); or organs of the chest (thoracoscopy and mediastinoscopy).

In certain embodiments, the method provides for treating a patient, wherein the patient is suffering from a disease related to type 2 inflammatory responses (e.g., asthma, allergy), the method comprising the steps of determining whether the patient expresses a gene signature, biological program or marker gene as described herein: obtaining or having obtained a biological sample from the patient; and performing or having performed an assay as described herein on the biological sample to determine if the patient expresses the gene signature, biological program or marker gene; and if the patient has an inflammatory gene signature, biological program or marker gene, then administering the one or more agents to the patient in an amount sufficient to shift the phenotype to a homeostatic phenotype, and if the patient does not have an inflammatory gene signature, biological program or marker gene, then not administering the one or more agents to the patient, wherein a risk of having inflammatory symptoms is increased if the patient has an inflammatory gene signature, biological program or marker gene.

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, an instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc.) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854, 5,288,644, 5,324,633; 5,432,049, 5,470,710, 5,492,806, 5,503,980, 5,510,270, 5,525,464, 5,547,839, 5,580,732, 5,661,028, and 5,800,992, the disclosures of which are herein incorporated by reference, as well as International Patent Publication Nos. WO 95/21265, WO 96/31622, WO 97/10365, and WO 97/27317, and EP 373203; and EP 78 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated herein in its entirety for all purposes. When the cDNA microarrays are used, typical hybridization conditions are hybridization in 5×SSC plus 0.2% SDS at 65 C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

### Sequencing and Single Cell Sequencing

In certain embodiments, the invention involves targeted nucleic acid profiling (e.g., sequencing, quantitative reverse transcription polymerase chain reaction, and the like) (see e.g., Geiss G K, et al., Direct multiplexed measurement of gene expression with color-coded probe pairs. Nat Biotechnol. 2008 March; 26(3):317-25). In certain embodiments, a target nucleic acid molecule (e.g., RNA molecule), may be sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard, reference is made to Macosko et al., 2015, “Highly Parallel Genome-wide Expression Profiling of Individual Cells Using Nanoliter Droplets” Cell 161, 1202-1214; International Patent Application No. PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as Patent Application No. WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain embodiments, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

### Methods of Screening

A further aspect of the invention relates to a method for identifying an agent capable of modulating one or more phenotypic aspects of a cell or cell population as disclosed herein, comprising: a) applying a candidate agent to the cell or cell population; b) detecting modulation of one or more phenotypic aspects of the cell or cell population by the candidate agent, thereby identifying the agent. The phenotypic aspects of the cell or cell population that is modulated may be a gene signature or biological program specific to a cell type or cell phenotype or phenotype specific to a population of cells (e.g., an inflammatory phenotype or suppressive immune phenotype). In certain embodiments, steps can include administering candidate modulating agents to cells, detecting identified cell (sub)populations for changes in signatures, or identifying relative changes in cell (sub) populations which may comprise detecting relative abundance of particular gene signatures.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of an immune cell or immune cell population may be modulated without substantially altering other (unintended, undesired) phenotypic aspect(s).

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

The methods of phenotypic analysis can be utilized for evaluating environmental stress and/or state, for screening of chemical libraries, and to screen or identify structural, syntenic, genomic, and/or organism and species variations. For example, a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value. By exposing cells, or fractions thereof, tissues, or even whole animals, to different members of the chemical libraries, and performing the methods described herein, different members of a chemical library can be screened for their effect on immune phenotypes thereof simultaneously in a relatively short amount of time, for example using a high throughput method.

Aspects of the present disclosure relate to the correlation of an agent with the spatial proximity and/or epigenetic profile of the nucleic acids in a sample of cells. In some embodiments, the disclosed methods can be used to screen chemical libraries for agents that modulate chromatin architecture epigenetic profiles, and/or relationships thereof.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures or biological programs of the present invention may be used to screen for drugs that reduce the signature or biological program in cells as described herein. The signature or biological program may be used for GE-HTS. In certain embodiments, pharmacological screens may be used to identify drugs that are selectively toxic to cells having a signature.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a signature or biological program of the present invention in silico.

The invention is further described in the following examples, which do not limit the scope of the invention described in the claims.

### EXAMPLES

### Example 1—CLEC-2^{-/-} Mice Develop Spontaneous Lung Inflammation

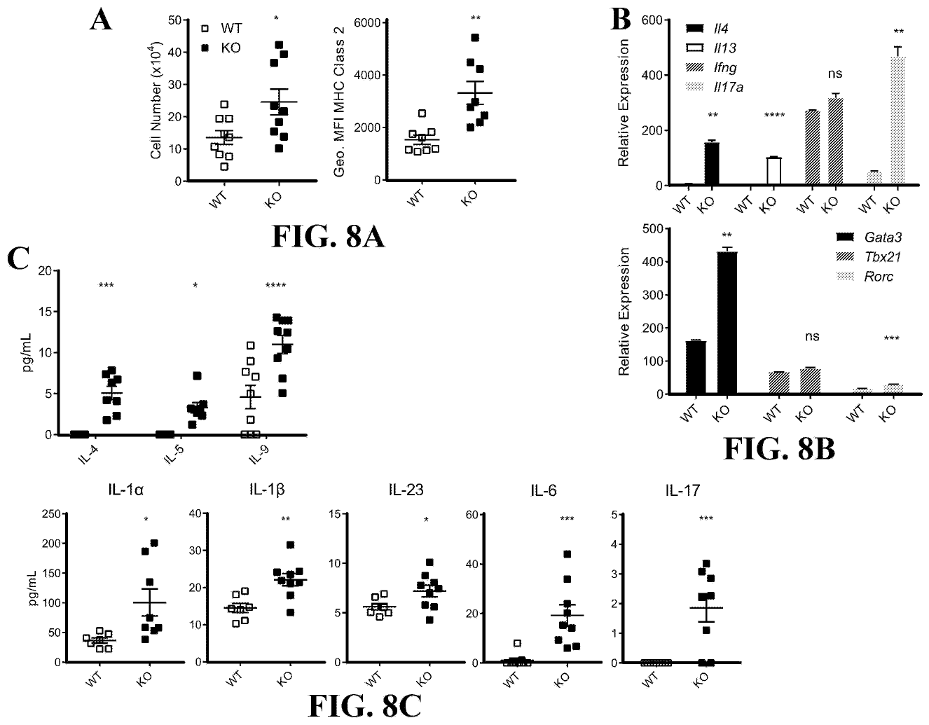

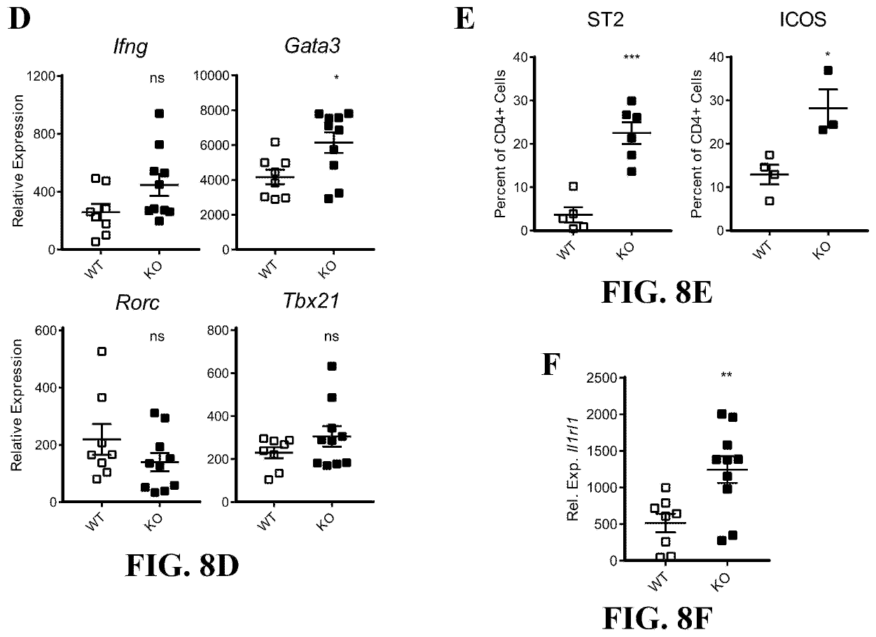

While CLEC-2 is one of several ligands known to interact with PDPN, PDPN is the only known endogenous ligand for CLEC-2, prompting us to analyze the role of CLEC-2 in lung homeostasis. Applicants observed spontaneous peri-bronchial inflammatory infiltrates in the lungs of CLEC-2^{-/-} mice, but not CLEC-2^{+/-} littermates (FIG. 1A). Moreover, CLEC-2^{-/-} mice also developed spontaneous goblet cell hyperplasia, as shown by PAS staining (FIG. 1A), and CLEC-2^{-/-} lungs had higher lung inflammation scores (FIG. 1B). Consistent with these findings, CLEC-2^{-/-} mice had a significant increase in the number of lung-infiltrating CD45⁺ cells compared to littermate controls (FIG. 1C), developed spontaneous BAL eosinophilia (FIG. 1D), and had increased numbers of Ly6C^{hi} MHC class II⁺ inflammatory monocytes (FIG. 8A). Given the histologic evidence of spontaneous airway inflammation, Applicants also evaluated physiologic lung function in CLEC-2^{-/-} mice and found that they had increased airway hyperreactivity as demonstrated by elevated airway resistance after methacholine challenge (FIG. 1E). Thus, in the absence of CLEC-2, mice develop spontaneous and physiologically significant airway inflammation.

### Example 2—Dysregulation of Type 2 and 3 Responses in the Absence of CLEC-2

To better characterize the spontaneous airway inflammation seen in CLEC-2^{-/-} mice, Applicants analyzed the expression of a panel of 203 immunologically relevant genes in lung-infiltrating immune cells from CLEC-2^{-/-} mice or littermate controls by Nanostring. Of those genes differentially expressed at least 1.5 fold, there was substantial upregulation of cytokines and transcription factors characteristic of both type 2 and type 3 immunity (e.g. Gata3, Rorc, Il4, Il33, Il17f, I123r) in the lungs of CLEC-2^{-/-} mice (FIG. 2A). Elevated expression of genes crucial for type 2 (Gata3, Il4, Il13) and type 3 (Rorc, Il17a) immunity was confirmed by qPCR in lung-infiltrating cells from CLEC-2^{-/-} mice, while type 1 related genes (Tbx2I, Ifng) were expressed at levels similar to those observed in control mice (8 FIG. 8B). Consistent with what Applicants observed at the transcriptional level, multiple pro-inflammatory cytokines related to both type 2 (IL-4, IL-5, and IL-9) and type 3 immunity (IL-1, IL-6, IL-23, IL-17) were elevated in lung tissue homogenates from CLEC-2^{-/-} mice at the protein level (8 FIG. 8C). IL-33 was also elevated at the protein level in the lungs of CLEC-2^{-/-} mice (FIG. 2B).

Given the central role of CD4 T cells in coordinating immune responses, Applicants examined the phenotype of lung-resident CD4 T cells in CLEC-2^{-/-} mice. The frequencies of CD4 T cells expressing the activation markers CD69 and CD44 were increased in the lungs of CLEC-2^{-/-} mice, consistent with a spontaneous acutely activated phenotype (FIG. 2C). Applicants therefore analyzed cytokine production by lung-resident CD4 T cells using intracellular cytokine staining. The frequency of CD4 T cells producing IL-5, IL-13, and IL-17A upon ex vivo stimulation was significantly increased in CLEC-2^{-/-} mice, while the frequency of IFNγ⁺ cells was similar to littermate controls (FIG. 2D). Additionally, Applicants sorted CD4 T cells from the lungs of CLEC-2^{-/-} mice or heterozygous littermate controls and directly analyzed expression of transcription factors and cytokines of different CD4 T cell subsets ex vivo. Th2-related cytokines and transcription factors (I14, 115, 1113, and Gata3) were consistently increased in CD4 T cells from CLEC-2^{-/-} mice, while ex vivo expression of Il17a, Ifng, Rorc, and Tbx2I was similar to controls (FIG. 2E, FIG. 8D). Finally, an increased frequency of lung- and BAL-resident CD4 T cells from CLEC-2^{-/-} mice expressed ICOS and ST2, the latter consistent with an increase in tissue-infiltrating Th2 cells (FIG. 2F, FIG. 8E, F). Therefore, pulmonary Th2 responses and, to a lesser extent, Th17 responses are specifically dysregulated in the absence of CLEC-2.

### Example 3—Altered Alveolar Macrophage Homeostasis in the Absence of CLEC-2

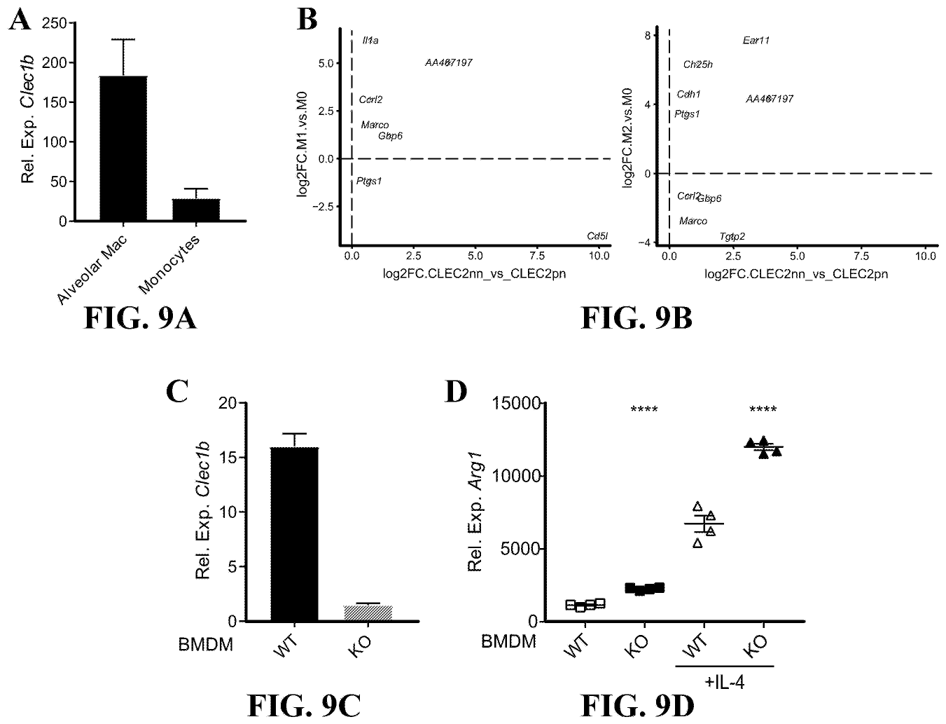

CLEC-2 has been reported to be expressed by a number of myeloid cell types and is also highly expressed by platelets (16, 17). To explore the cellular mechanisms responsible for the spontaneous type 2 lung inflammation Applicants observed in CLEC-2-deficient mice, Applicants analyzed expression of CLEC-2 in various lung-resident cell types. Applicants sorted multiple lung-resident cell populations and analyzed expression of CLEC-2 (Clec1b) by qPCR. CLEC-2 expression was primarily noted in alveolar macrophages, with minimal CLEC-2 expression seen in the other populations (FIG. 3A, FIG. 9A), consistent with publicly available gene expression databases (18).

Since CLEC-2 was preferentially expressed by alveolar macrophages, Applicants further analyzed the phenotype of these cells in CLEC-2^{-/-} mice. Notably, CLEC-2^{-/-} alveolar macrophages were markedly reduced both by frequency (FIG. 3B) and absolute number (FIG. 3C), indicating alveolar macrophage homeostasis is altered in the absence of CLEC-2. Moreover, despite their reduction in number and frequency, the remaining CLEC-2^{-/-} alveolar macrophages expressed increased levels of MHC class II compared to controls (FIG. 3D). To understand the mechanisms by which CLEC-2 regulates alveolar macrophage function, Applicants undertook RNA-seq to assess the global changes in gene expression in alveolar macrophages between wildtype and CLEC-2^{-/-} mice (FIG. 3E). Consistent with the earlier analysis, alveolar macrophages from CLEC-2^{-/-} mice had elevated expression of multiple genes related to antigen presentation compared to littermate controls (H2-Q9, H2-DMb1, H2-Q4 and H2-M2), suggesting that the remaining macrophages in the CLEC-2^{-/-} mice exhibit an activated phenotype. Moreover, expression of the pro-inflammatory cytokine Il1a and several genes encoding either chemokines or chemokine receptors (Ccr5, Ccl9, and Cxcl16) was also elevated, while expression of the anti-inflammatory cytokine Tgfb2 was reduced in CLEC-2^{-/-} alveolar macrophages. Applicants also compared the genes that were differentially expressed in CLEC-2^{-/-} alveolar macrophages (compared to wild type) with those genes associated with either an M1 or M2 macrophage phenotype in a recent study (19). Applicants found that CLEC-2^{-/-} alveolar macrophages had increased expression of genes previously shown to be upregulated by both in-vitro-derived M1 macrophages (Gbp6, Irf5, Il1a and Marco) and alternatively activated M2 macrophages (Cd5l, AA467197, Ch25h and Cdh1) (FIG. 3E, FIG. 9B). Additionally, CLEC-2^{-/-} alveolar macrophages had increased expression of the M2-associated gene Arg1 by qPCR (FIG. 3F). Overall, in the absence of CLEC-2, alveolar macrophages are reduced in number but demonstrate a unique, spontaneously activated phenotype, with features of both M1 and M2 macrophages.

Since CLEC-2^{-/-} mice have been shown previously to have abnormal lymphatic development, which could in turn impact the phenotype of alveolar macrophages, Applicants also analyzed the phenotype of bone marrow derived macrophages (BMDMs) from CLEC-2^{-/-} mice. CLEC-2 was expressed by wildtype BMDMs but not CLEC-2^{-/-} BMDMs (FIG. 9C), and CLEC-2^{-/-} BMDMs also demonstrated increased expression of Arg1 at baseline which was further exaggerated after stimulation with IL-4 (FIG. 9D). Therefore, CLEC-2 regulates alternative macrophage activation independently of its role in lymphatic development.

### Example 4—CLEC-2^{-/-} Myeloid Cells Amplify Th2 Differentiation and Activation

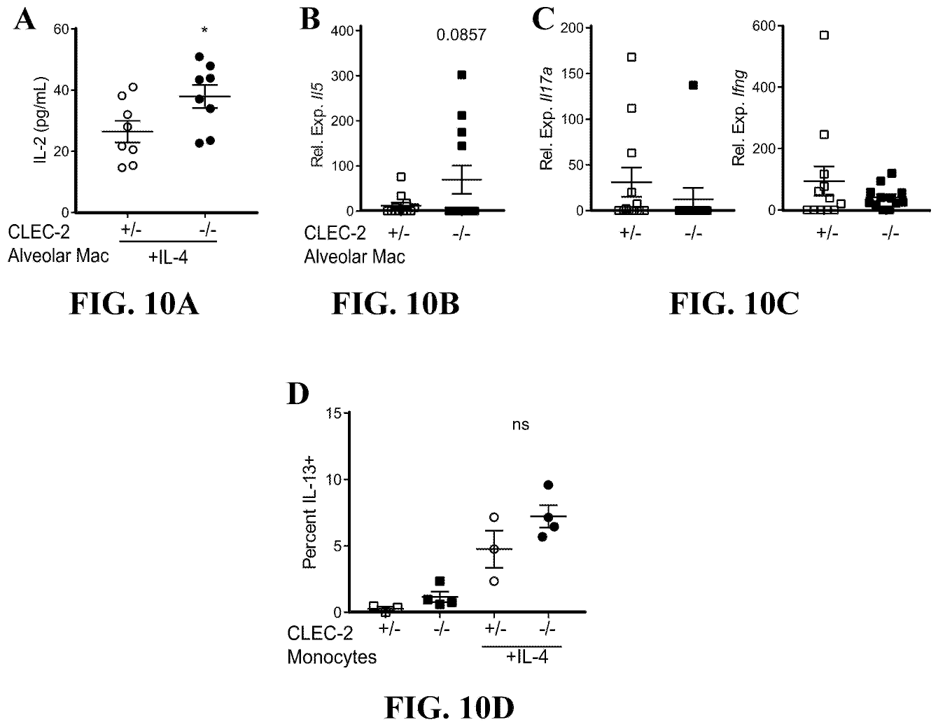

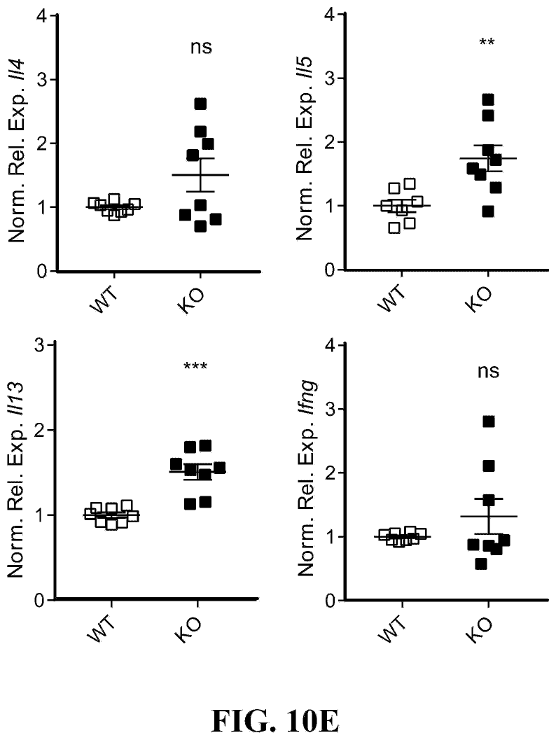

Applicants next investigated whether lung-resident CLEC-2^{-/-} myeloid cells differed in their ability to activate CD4 T cells in vitro. MHC class II^{hi} monocytes are increased in number in CLEC-2^{-/-} mice; however these monocytes normally have lower expression of CLEC-2 compared to alveolar macrophages. Applicants isolated both alveolar macrophages and lung-resident MHC class II^{hi} monocytes from CLEC-2^{-/-} mice or wildtype littermates and cultured them with naïve wildtype CD4 T cells. Under non-biased T cell priming conditions (anti-CD3 alone), Applicants observed no difference in the ability of either alveolar macrophages or monocytes from the wild type CLEC-2^{+/-} or CLEC-2^{-/-} mice to promote T cell proliferation, as shown by CellTrace Violet dilution (FIG. 4A). However, under Th2 conditions, CLEC-2^{-/-} alveolar macrophages specifically enhanced T cell proliferation, in contrast to monocytes, where proliferation was similar regardless of CLEC-2 genotype (FIG. 4A). The increased proliferation was correlated with increased production of IL-2 from T cells cultured with CLEC-2^{-/-} alveolar macrophages (FIG. 10A). Therefore, CLEC-2 appears to specifically regulate the ability of alveolar macrophages to promote CD4 T cell proliferation in the context of a type 2 immune response.

Applicants then examined expression of effector cytokines in CD4 T cells cultured with wildtype or CLEC-2^{-/-} alveolar macrophages. Under unbiased conditions, there was a trend to increased expression of 115 in T cells cultured with CLEC-2^{-/-} alveolar macrophages, but this did not reach statistical significance (FIG. 10B). Applicants also examined expression of both Ifng and Il17a, and expression of these cytokines was similar regardless of the genotype of the alveolar macrophages (FIG. 10C). However, similar to what was seen for T cell proliferation, CLEC-2^{-/-} alveolar macrophages significantly enhanced type 2 cytokine production by CD4 T cells in the presence of exogenous IL-4 (FIG. 4B, C). Notably, CLEC-2^{-/-} MHC class II^{hi} monocytes did not significantly enhance type 2 cytokine expression in T cells, indicating this was specific to alveolar macrophages (FIG. 10D). Therefore, loss of CLEC-2 expression results in alveolar macrophages that promote Th2 proliferation and effector function.

Since CLEC-2^{-/-} mice develop spontaneous lung inflammation which could secondarily alter the ability of alveolar macrophages to prime CD4 T cells, Applicants also performed T cell co-culture experiments with BMDMs derived from either CLEC-2^{-/-} mice or wildtype littermates. Similar to what Applicants observed for alveolar macrophages, T cells co-cultured with CLEC-2^{-/-} BMDMs also expressed higher levels of type 2 cytokines compared to wildtype (FIG. 10E). Notably, CD4 T cells co-cultured with CLEC-2^{-/-} BMDMs did not have elevated expression of Ifng, just as expression of Th1-related genes was similar in the lungs of CLEC-2^{-/-} mice. These data therefore suggest that CLEC-2 expression by myeloid cells is particularly important for regulating type 2 immune responses.

### Example 5—Myeloid Specific Deletion of CLEC-2 Promotes Type 2 Immunity

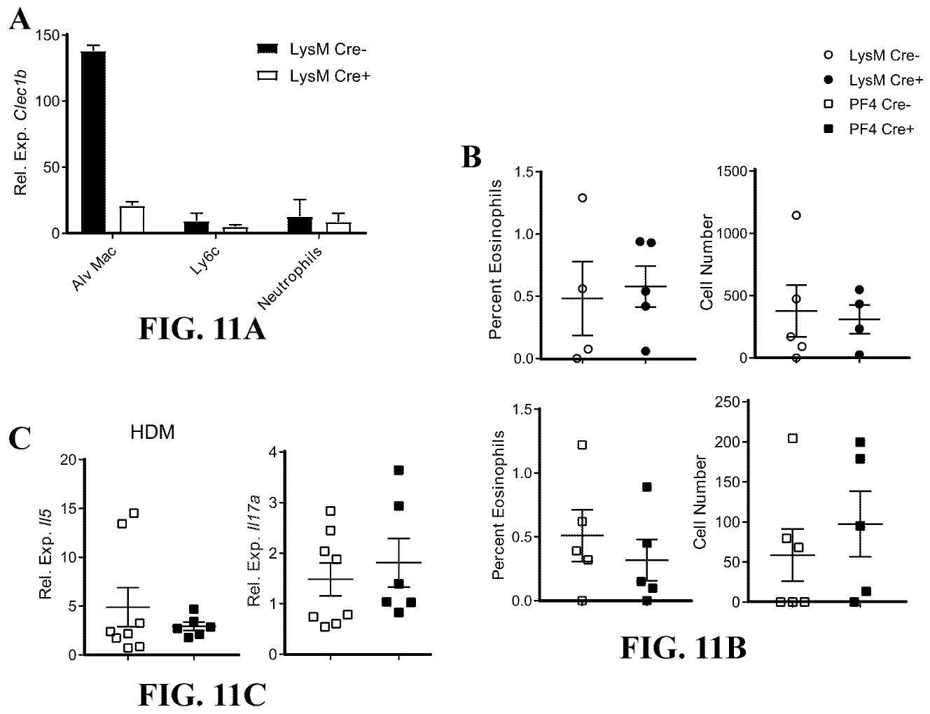

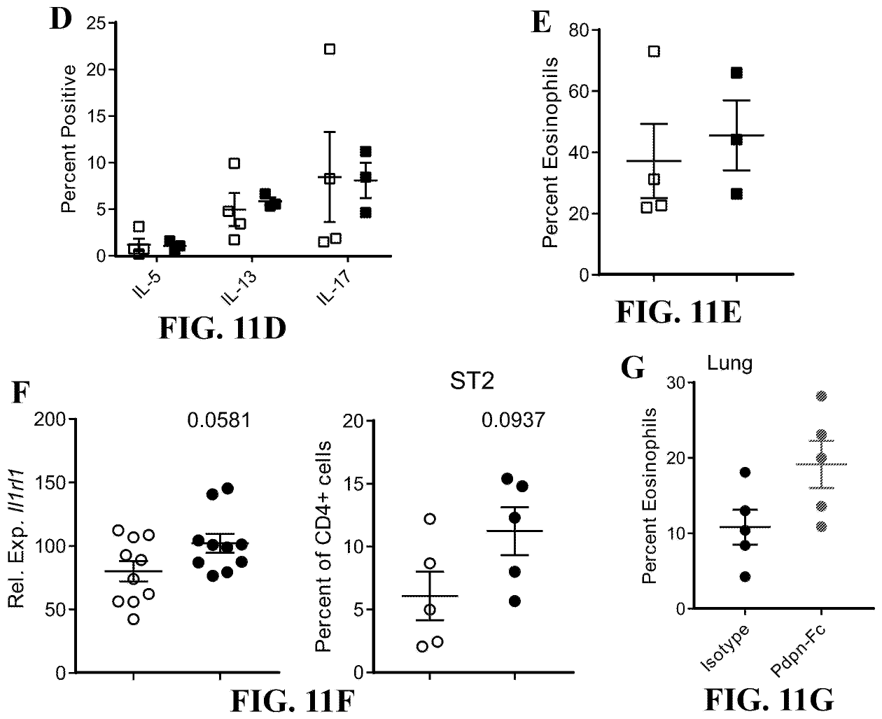

This data suggests that CLEC-2 is primarily expressed by alveolar macrophages, in contrast to other lung resident myeloid populations, and that it is critical to regulating type 2 immune responses in vivo and in vitro. However, CLEC-2 is also highly expressed by platelets, where it also plays a role in promoting normal lymphatic development, which could, in turn, also impact regulation of pulmonary immune responses. Given this, Applicants investigated the impact of cell-type-specific CLEC-2 deletion on pulmonary immune homeostasis, particularly the regulation of type 2 immune responses. Applicants crossed CLEC-2^{fl/fl} mice to mice expressing Cre recombinase in either the myeloid compartment (using LysM-Cre mice) or in platelets (using PF4-Cre mice). CLEC-2 expression was specifically reduced in the alveolar macrophages of CLEC-2^{fl/fl} LysM-Cre mice (FIG. 11A). While Applicants did not observe spontaneous eosinophilia in either CLEC-2¹¹ LysM-Cre or CLEC-2^{fl/fl} PF4-Cre mice (FIG. 11B), Applicants did note that expression of the key type 2 cytokines 114 and 1113 was significantly increased at steady state in CLEC-2^{fl/fl} LysM-Cre mice, but not in PF4-Cre mice, indicating low level dysregulation of type 2 responses following myeloid specific deletion of CLEC-2 (FIG. 5A). In contrast, expression of Il17a was significantly increased in both CLEC-2^{fl/fl} LysM-Cre or CLEC-2^{fl/fl} PF4-Cre mice (FIG. 5A), suggesting that CLEC-2 expression by both myeloid cells and platelets is important for controlling type 3 responses in vivo.

To investigate if allergen challenge could exacerbate the low-level dysregulation of type 2 immunity already present in CLEC-2^{fl/fl} LysM-Cre mice, Applicants challenged CLEC-2^{fl/fl}, CLEC-2^{fl/fl} LysM-Cre, or CLEC-2^{fl/fl} PF4-Cre mice with house dust mite extract (HDM), a well described aero-allergen in both humans and mice. Applicants did not observe significant differences in cytokine expression or lung-infiltrating eosinophils between CLEC-2^{fl/fl} PF4-Cre mice and Cre⁻ littermates following HDM challenge, demonstrating that platelet expression of CLEC-2 was not required for regulating responses to aero-allergen (FIG. 11C-E). In contrast, the frequency of BAL eosinophils was significantly increased in CLEC-2^{fl/fl} LysM-Cre mice (FIG. 5B), and there was a small but significant increase in CD44^{hi} CD4 T cells in the BAL (FIG. 5C). Expression of Il4, Il5, and 1113 in lung infiltrating leukocytes from CLEC-2^{fl/fl} LysM-Cre was also increased (FIG. 5D), indicating that myeloid specific expression of CLEC-2 is critical for regulating allergen-induced type 2 responses. Similar to what Applicants observed in global CLEC-2^{-/-} mice, CLEC-2^{fl/fl} LysM-Cre mice had reduced numbers of alveolar macrophages following allergen challenge (FIG. 5E). Moreover, loss of CLEC-2 in myeloid cells resulted in enhanced expression of Il33 following allergen challenge (FIG. 5F), and there was a trend towards increased expression of ST2 in CD4 T cells in both the lung and BAL (FIG. 11F). Applicants also examined expression of Il33 after allergen challenge in mice lacking PDPN in lung epithelial cells (PDPN^{fl/fl} Nkx2.1-Cre mice). Compared to PDPN^{fl/fl} littermate controls, PDPN^{fl/fl} Nkx2.1-Cre mice had increased Il33 expression after HDM challenge (FIG. 5G), similar to what Applicants observed in CLEC-2^{fl/fl} LysM-Cre mice. Therefore, myeloid cell expression of CLEC-2 regulates type 2 immune responses in the lung, in part by controlling expression of the alarmin IL-33 via interactions with PDPN on epithelial cells.

### Example 6—Disruption of Endogenous CLEC-2/PDPN Interactions Enhances Type 2 Responses

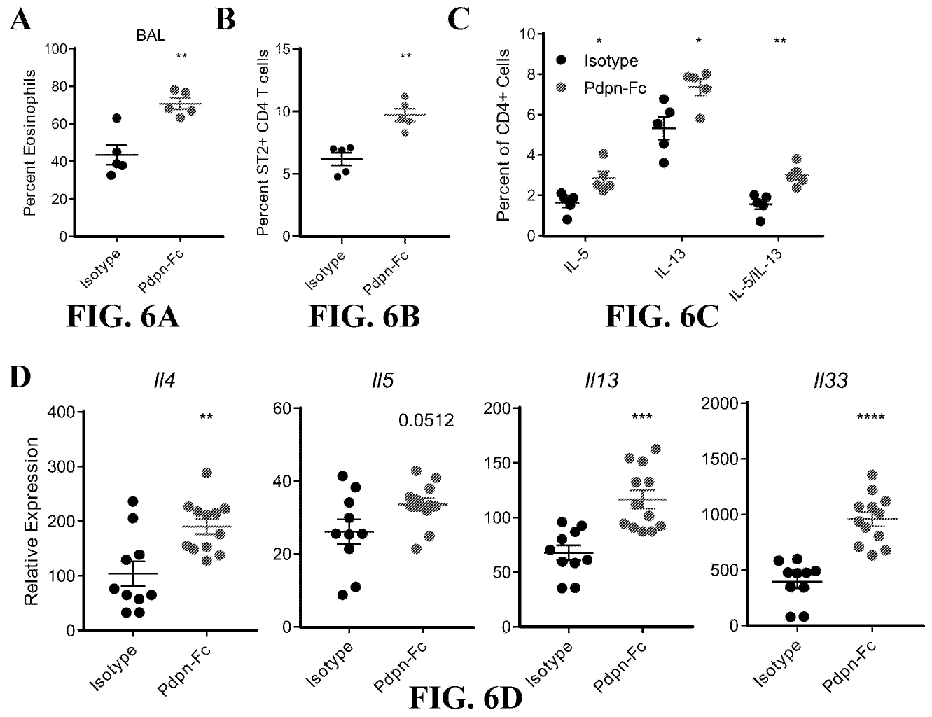

Since deletion of CLEC-2 during embryogenesis may result in developmental defects that contribute to the phenotype Applicants observed, Applicants sought to disrupt CLEC-2/PDPN interactions in adult mice using a recombinant PDPN-Fc fusion protein that has previously been shown to inhibit the interaction of CLEC-2 and PDPN (20). Applicants administered intranasal HDM along with either PDPN-Fc or an isotype control to wildtype C57BL/6J mice and assessed cellular and molecular readouts of allergic inflammation. Similar to the observations in mice with myeloid specific deletion of CLEC-2, mice treated with PDPN-Fc had an increased frequency of eosinophils in the BAL and a trend towards an increased frequency of lung-infiltrating eosinophils (FIG. 6A, FIG. 11G). Moreover, the frequency of ST2⁺ CD4 T cells was increased in PDPN-Fc treated mice, as was the frequency of lung-resident CD4 T cells positive for IL-5 and IL-13 by intracellular cytokine staining, indicating an increased Th2 response (FIG. 6B, C). Supporting this finding, there was significantly increased expression of Il4 and 1113, along with a trend towards increased 115 in lung-infiltrating leukocytes, as assessed by qPCR (FIG. 6D). Expression of the alarmin Il33 was significantly increased in PDPN-Fc treated mice (FIG. 6D) further highlighting the importance of endogenous CLEC-2/PDPN interactions in regulating IL-33 expression.

### Example 7—Dysregulated IL-33 Expression Promotes Type 2 Immunity in CLEC-2^{-/-} Mice

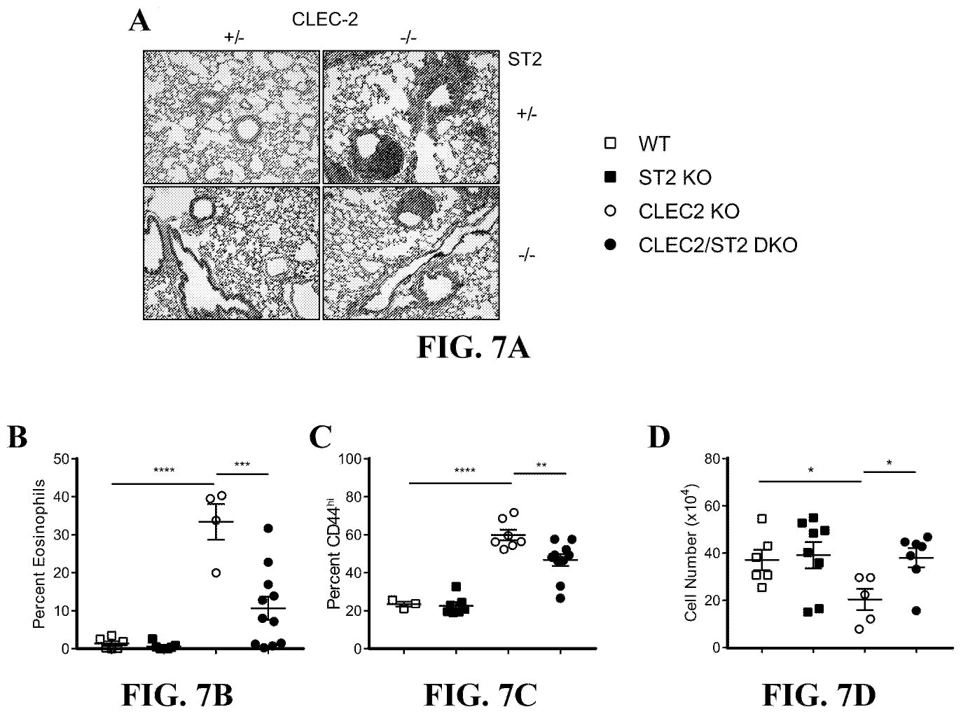

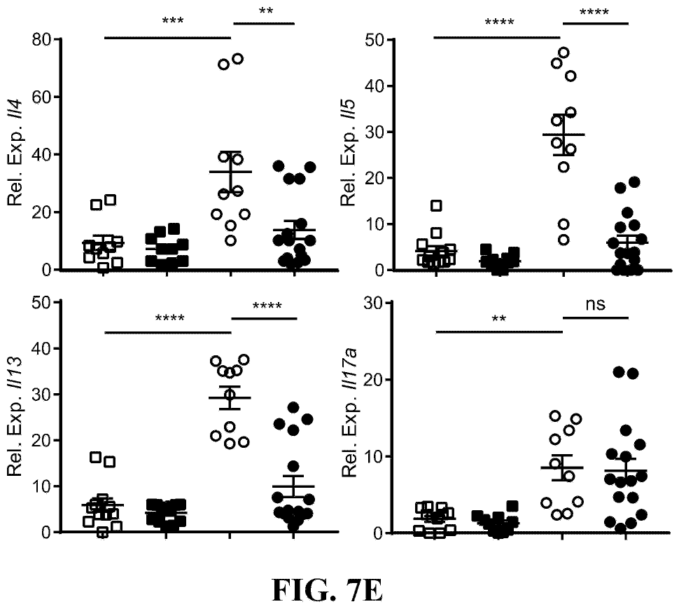

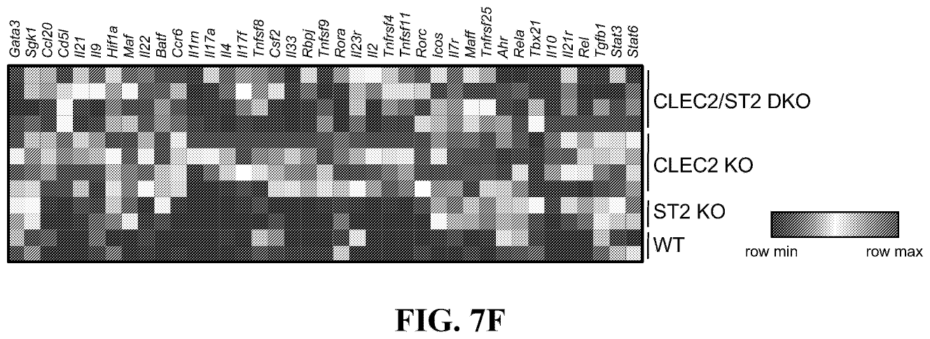

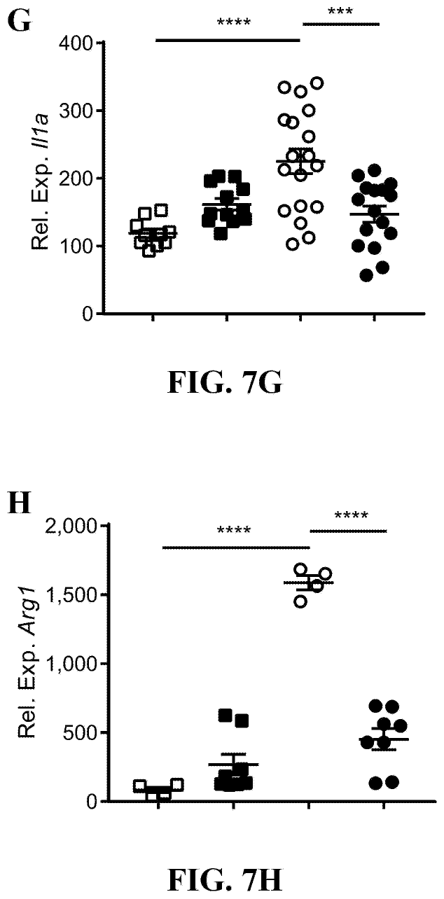

Having found that CLEC-2 expression on myeloid cells regulates Th2 responses both in vivo and in vitro, Applicants then investigated the signals downstream of CLEC-2 that drive dysregulation of type 2 responses in CLEC-2^{-/-} mice in vivo. Since Applicants observed increased expression of both IL-33 and its receptor, ST2, following disruption of CLEC-2/PDPN interactions, Applicants hypothesized that the spontaneous lung inflammation seen in CLEC-2^{-/-} mice could be driven by dysregulation of this alarmin. Applicants therefore generated CLEC-2^{-/-} ST2^{-/-} (DKO) mice and analyzed whether loss of IL-33-signaling altered the spontaneous lung inflammation seen in CLEC-2^{-/-} mice. In contrast to mice deficient in CLEC-2, histologic analysis highlighted that DKO mice had reduced peribronchial infiltrates and goblet cell hyperplasia, though they still developed peri-vascular lymphocytic infiltrates (FIG. 7A). Consistent with the decreased histologic airway inflammation, Applicants also observed a reduced frequency of eosinophils in the BAL and a reduced frequency of CD44^{hi} CD4 T cells, although both were increased compared to wildtype or ST2^{-/-} mice (FIG. 7B, C). Moreover, Applicants observed similar frequencies of alveolar macrophages in DKO mice compared to wildtype or ST2^{-/-} mice, suggesting that the reduction in macrophage frequency in CLEC-2^{-/-} mice was secondary to IL-33 driven spontaneous airway inflammation (FIG. 7D). Expression of the type 2 cytokines Il4, Il5, and Il13 in lung-infiltrating immune cells was also reduced in DKO mice compared to mice deficient only in CLEC-2 (FIG. 7E). In contrast, expression of Il17a was similarly elevated in the lungs of CLEC-2^{-/-} and DKO mice, suggesting that in the absence of CLEC-2, dysregulated IL-33 expression specifically promotes aberrant type 2 immune responses.

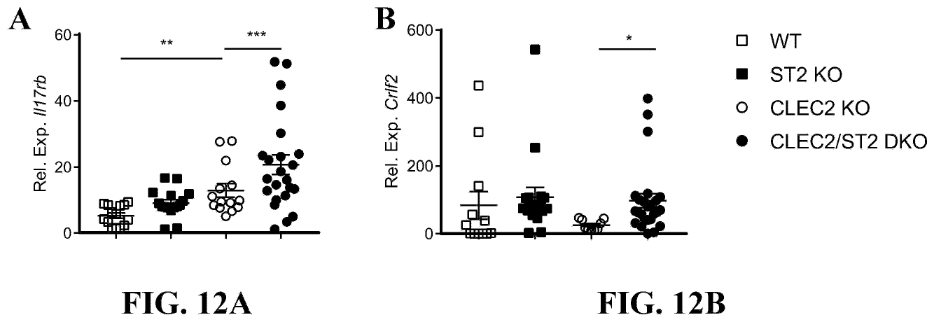

These results, coupled with the observations that DKO mice still had increased BAL eosinophils and peri-vascular infiltrates compared to wildtype controls, suggested the possibility that other type 2 alarmins may also be dysregulated in the absence of CLEC-2. Consistent with this hypothesis, Applicants observed that expression of Il17rb, the specific receptor for IL-25, was increased in total lung-infiltrating immune cells in CLEC-2^{-/-} mice and was further elevated in DKO mice (FIG. 12A). While expression of Crlf2, the unique receptor chain for TSLP, was also elevated in DKO mice compared to CLEC-2^{-/-} mice, expression was similar to that observed in ST2^{-/-} mice, suggesting that the increased expression seen in DKO mice was compensatory for the loss of IL-33 signaling, rather than a result of loss of CLEC-2 (FIG. 12B). Despite the evidence of compensatory upregulation of other alarmins in the absence of IL-33-mediated signals, when Applicants assessed expression of a panel of genes whose expression was elevated in CLEC-2-lung-infiltrating leukocytes, Applicants found that many were significantly reduced in the absence of ST2 (FIG. 7F). Additionally, the elevated expression of Il1a and Arg1 seen in CLEC-2^{-/-} mice was nearly entirely dependent upon IL-33 signaling (FIG. 7G, H). Thus, CLEC-2 plays a key role in regulating expression of IL-33, and the absence of IL-33-mediated signals substantially mitigates the spontaneous lung inflammation seen in CLEC-2^{-/-} mice.

### Example 8—Conclusion

This work highlights a molecular pathway that regulates type 2 immune responses in the lung, whereby expression of CLEC-2 by alveolar macrophages regulates expression of the epithelial alarmin IL-33. Tissue-based type 2 responses are critically dependent upon alarmins, such as IL-33, which primarily are produced by non-hematopoietic cells. While alarmins are known to be released after stress or injury, the molecular pathways regulating their production have remained unclear. Via their close interactions with epithelial cells, alveolar macrophages are well positioned to modulate production of epithelial-derived alarmins. The only known endogenous ligand of CLEC-2 is PDPN, which is highly expressed by several CD45− cell populations in the lung, including epithelial cells (18). This, coupled with the spontaneous type 2 lung inflammation Applicants observe in CLEC-2^{-/-} mice, lead Applicants to hypothesize that interactions between CLEC-2 on myeloid cells and PDPN on epithelial cells represent a tissue-based checkpoint that controls production of IL-33 under homeostatic conditions. Importantly, several studies have highlighted that acute inflammation leads to loss of the pre-existing alveolar macrophage population, which are eventually replaced by monocyte-derived macrophages (21, 22). In contrast to alveolar macrophages, CLEC-2 is not highly expressed by inflammatory monocytes, which are rapidly recruited to sites of acute inflammation. Therefore, in this setting, tonic CLEC-2/PDPN signals would be diminished, resulting in increased expression of IL-33 after epithelial injury or stress. Such a model would be consistent with the idea that tonic signals from healthy tissues down-modulate pro-inflammatory responses by tissue macrophages at steady state, in response to environmental stimuli, and that disruption of these interactions is a key event in promoting tissue inflammation, similar to what has been demonstrated for surfactant in regulating alveolar macrophage function (23, 24).

This data also raises the question of whether PDPN directly activates intracellular pathways that regulate IL-33 expression, or if instead PDPN binding to CLEC-2 elicits changes in alveolar macrophage gene expression and function, thereby indirectly modulating epithelial cell gene expression. Supporting a potential indirect pathway, PDPN has a short cytoplasmic tail without well-described signaling motifs, whereas CLEC-2 activates Syk via a hemi-ITAM motif. Moreover, activation of CLEC-2 has been described to alter the response of BMDMs to LPS, decreasing IL-12 production and promoting expression of IL-10, suggesting that CLEC-2 preferentially promotes an anti-inflammatory macrophage phenotype (25). Consistent with this finding, Applicants observe increased expression of IL-1α by CLEC-2^{-/-} alveolar macrophages (FIG. 3F), levels of IL-1α are increased in the lungs of CLEC-2^{-/-} mice (FIG. 8C), and IL-1α has been shown to promote release of IL-33 by bronchial epithelial cells after allergen challenge (26), suggesting at least one potential indirect pathway by which CLEC-2 may regulate IL-33 production.

Applicants and others have found that Th17 cells express PDPN (13, 27, 28), raising the question of whether CD4 T cell expression of PDPN modulates macrophage function. This is particularly relevant given that the results show that CLEC-2 deficient alveolar macrophages directly enhance Th2 proliferation and effector function in vitro, independent of their role in modulating epithelial cell alarmin expression. Importantly, this effect is most pronounced in the presence of exogenous IL-4, and Applicants have found that Th2 cells, unlike Th17 cells, do not significantly express PDPN (27). Moreover, Applicants have not observed spontaneous type 2 responses in Pdpn^{fl/fl} CD4-Cre mice, indicating that PDPN expression by T cells is dispensable for CLEC-2-mediated regulation of type 2 immune responses (data not shown). However, given that Applicants also observe increased expression of type 3 cytokines in the lungs of CLEC-2^{-/-} mice, it remains possible that PDPN expression on CD4 T cells may play a role in regulating type 3 inflammation.

Importantly, while Applicants find that CLEC-2 is primarily expressed by alveolar macrophages, and loss of CLEC-2 expression specifically in the myeloid compartment leads to amplified type 2 responses in tissues following aero-allergen exposure, Applicants do not observe spontaneous lung inflammation in CLEC-2^{fl/fl} LysM-Cre mice, in contrast to mice globally deficient in CLEC-2. This suggests that expression of CLEC-2 by non-myeloid cells also plays a critical immunoregulatory role. Supporting this, Applicants observe increased type 3 cytokine production in mice lacking CLEC-2 specifically in platelets, and others have noted that platelet expression of CLEC-2 regulates both LPS-induced acute lung inflammation and sepsis (29, 30). Moreover, expression of CLEC-2 on platelets is critical for normal lymphatic development, and maintenance of normal lymphatics has been shown to promote immune tolerance in animal models of lung transplant (31). Finally, evidence has emerged highlighting a role for CLEC-2 on platelets interacting with PDPN on lymphatic endothelial cells in regulating TGFβ release (32). While this application highlighted the role of this pathway in lung development, given the pleiotropic role of TGFβ, it could also represent an immunoregulatory pathway. Therefore, CLEC-2 appears to regulate immune responses via a number of distinct cellular and molecular mechanisms.

Finally, while the exact mechanisms by which loss of CLEC-2 gives rise to alveolar macrophages that enhance Th2 responses remains to be elucidated, differential gene expression in alveolar macrophages from CLEC-2^{-/-} mice shows that they upregulate genes associated with both M1 and M2 macrophages. Among these genes, CLEC-2^{-/-} alveolar macrophages notably express higher levels of Irf5 (FIG. 3B), and IRF5⁺ monocyte-derived macrophages have been shown to promote Th1 and Th17, but not Th2 cell differentiation in vitro (33). Although asthma is often thought of as mediated by dysregulated type 2 inflammation, recent work has highlighted that some patients also demonstrate dysregulated type 3 immunity, either alone or in combination with aberrant type 2 responses (4, 34), and the presence of mixed type 2 and type 3 asthma is correlated with increased disease severity (34, 35). Moreover, alveolar macrophages from asthmatics have features of both M1 and M2 macrophages, including increased numbers of IRF5⁺ macrophages, and treatment with inhaled corticosteroids decreased this population (36). Given that both type 2 and 3 responses are dysregulated in the lungs of CLEC-2^{-/-} mice, the data suggest that dysregulation of CLEC-2 signaling may contribute to the pathobiology of severe asthma, and that potentiating CLEC-2 signaling in alveolar macrophages could represent a novel means for inhibiting mixed type 2/3 inflammatory responses in this clinical context.

### Example 9—Methods

Mice: C57BL/6J, C57BL/6-Tg(Pf4-icre)Q3Rsko/J (PF4-Cre), B6.129P2-Lyz2tm1(cre)Ifo/J (LysM-Cre), and C57BL/6J-Tg(Nkx2-1-cre)2Sand/J (Nkx2.1-Cre) mice were from the Jackson Laboratory. CLEC-2^{-/-} mice on 129/Sv background were provided by Shannon Turley (Dana-Farber Cancer Institute, Boston, Mass., USA) and backcrossed with C57BL/6J for 5 generations in house. CLEC-2^{fl/fl} mice on 129/Sv background were provided by Mark Kahn (University of Pennsylvania, Philadelphia, Pa., USA) and crossed with PF4-Cre or LysM-Cre in house. PDPN^{fl/fl} mice were generated as previously described (13) and crossed to Nkx2.1-Cre mice in house. Il1rl1^{-/-} mice on C57BL/6J background were originally generated by Andrew McKenzie and provided by Diane Mathis (Harvard Medical School, Boston, Mass., USA). To induce allergic airway inflammation, mice were treated intranasally with 10 μg house dust mite (HDM) extract (Greer Laboratories) on day 0, 7, 8, and 9, and then euthanized on day 10. For some experiments 25 μg Podoplanin-Fc fusion protein (PDPN-Fc) or isotype control (Biolegend) was administered intranasally on the same day as HDM. All mice were maintained in specific pathogen-free conditions at the Hale Building of Transformative Medicine. All animal experiments were performed in compliance with the approved BWH IACUC protocols.

Preparation of cell suspensions: Mice were euthanized with CO₂. Bronchoalveolar lavage (BAL) was performed with one wash of 1.5 ml cold PBS using a secured tracheal cannula. BAL fluid was centrifuged, and cell pellet was resuspended for counting and flow cytometry. Lung lobes were removed after BAL. The post-caval lobe was fixed in 10% buffered formalin for histology, and remaining lobes were processed into single cell suspensions via the GentleMACS lung dissociation kit (Miltenyi Biotec) per the manufacturer's instructions. Lung supernatant was collected for cytokine analysis. Bone marrow cells were isolated from femurs and tibias. Erythrocytes were lysed with ACK buffer and then the cell suspension was filtered through a 70 μM cell strainer. Live cells were counted using the Precision Count Beads (BioLegend) or a hemocytometer following Trypan Blue staining (Sigma-Aldrich). T cells were isolated from spleen and lymph nodes. Following ACK lysis, single cell suspensions were incubated with CD4 MicroBeads (Miltenyi BioTec) on ice and enriched for CD4⁺ cells via magnetic separation with LS column per manufacturer's instructions.

Antibodies: The following antibodies were used for flow cytometry analysis: CD3R (clone:145-2C11), CD4 (clone: RM4-5), CD8a (clone: 53-6.7), CD11b (clone: M1/70), CD11c (clone: N418), CD19 (clone: 6D5), CD41 (clone: MWReg30), CD44 (clone: IM7), CD45 (clone: 30-F11), CD69 (clone: H1.2F3; BD Pharmigen), CD90.2 (clone: 30-H12), CD127 (clone: A7R34), CLEC-2 (clone: 17D9), F4/80 (clone: BM8), I-A/I-E (clone: M5/114.15.2), Icos (clone: C398.4A), IFNγ (clone: XMG1.2), IL-5 (clone: TRFK5), IL-13 (clone: eBio13A; eBioscience), IL-17A (clone: Tc11-18H10.1), Ly-6C (clone: HK1.4), Ly-6G (clone: 1A8; BD Pharmingen), NK1.1 (clone: PK136), Siglec-F (clone: E50-2440; BD Pharmigen), ST2 (IL1RL1; clone: DIH9), TCRβ (clone: H57-597), and TCRγδ (clone: GL3) were purchased from BioLegend unless otherwise specified.

Flow Cytometry: Data was obtained using an LSRFortessa and analyzed with FlowJo software (Tree Star). Live cells were distinguished using the live/dead marker eFluor506 (eBioscience). For intracellular cytokine staining, cells were stimulated with 50 ng ml⁻¹ phorbol 12-myristate 13-acetate (Sigma-Aldrich) and 1 μM ionomycin (Sigma-Aldrich) in the presence of monensin (GolgiStop, BD Pharmigen) or Cell Activation Cocktail with Brefeldin A (BioLegend) for 5 hours at 37 C°. Cells were then stained with the live/dead marker, then fixed and stained using the BD cytofix/cytoperm solution kit (BD Biosciences) according to the manufacturer's instructions. Different cell types were determined with following gating strategy: alveolar macrophages (Live⁺ CD45⁺ CD11b^{int}CD11chiSiglec-F⁺), inflammatory monocytes (Live⁺ CD45⁺ CD11b⁺Ly6G-Ly6C^{hi}), T cells (Live⁺ CD45⁺ Thy1.2⁺ CD4⁺), eosinophils (Live⁺ CD45⁺ CD11b⁺SSC^{hi} Siglec-F⁺), and dendritic cells (Live⁺ CD45⁺ CD11b⁺ CD11c⁺ CD103⁺).

Fluorescence-activated cell sorting: Single cell suspensions of lung or spleen were generated as described above and erythrocytes lysed with ACK. Alveolar macrophage (Live⁺ CD45⁺ CD11b^{int}CD11c^{hi} Siglec-F⁺), MHC class II⁺ Monocytes (Live⁺ CD45⁺ CD11b⁺Ly6G-Ly6C^{hi} MHCII⁺), and MHC class II⁻ Monocytes (Live⁺ CD45⁺ CD11b⁺Ly6G-Ly6C^{hi} MHCII⁻) were sorted using a BD FACSAria (BD Biosciences). Naïve CD4⁺ T cells were identified as Live⁺ CD4⁺SSC^{lo}CD44⁻ CD25⁻ and sorted using a BD FACSAria (BD Biosciences).

In vitro Cell Culture: Bone marrow derived macrophages (BMDMs) were generated by culture in RPMI supplemented with 10 ng ml⁻¹ MC-SF (BioLegend) for 6 days. BMDMs were then stimulated with 20 ng ml⁻¹ IL-4 (Miltenyi Biotec) or 50 ng ml⁻¹ LPS (Enzo) for 6 to 48 hours, or as indicated. For co-culture experiments, BMDMs and naïve CD4⁺ T cells were cultured with 0.1 μg ml⁻¹ anti-CD3 (Bio X Cell) in a 48-well plate either with or without 20 ng ml⁻¹ IL-4 (Miltenyi Biotec). RNA and supernatants were collected after 4 days. Sorted alveolar macrophages, MHC class II⁺ monocytes, or MHC class II⁻ monocytes were co-cultured with naïve CD4⁺ T cells. Naïve CD4⁺ T cells were labelled with CellTrace Violet (Thermo Fisher Scientific) prior to co-culture and activated with 0.1 μg ml⁻¹ anti-CD3 (Bio X Cell) with or without 20 ng ml⁻¹ IL-4 (Miltenyi Biotec). After 4 days, supernatant and RNA were collected and T cells were stimulated as described above for intracellular cytokine staining.

Quantitative real-time PCR: Total RNA was isolated from samples using the RNeasy Plus Mini Kit (Qiagen), and then was reverse transcribed into cDNA using the iScript cDNA Synthesis Kit (Bio-Rad) according to the manufacturer's instructions. Quantitative real-time RT-PCR was performed on a ViiA7 System (Thermo Fisher Scientific) with TaqMan Fast Advanced Master Mix (Thermo Fisher Scientific) and purchased primer/probe sets: Gata3 (Mm00484683_m1), Tbx2I (Mm00450960_m1), Rorc (Mm01261022_m1), I14 (Mm00445259_m1), I/5 (Mm00439646_m1), I113 (Mm00434204_m1), Ifng (Mm01168134_m1), Il17a (Mm00439618_m1), Clec1b (Mm01183353_m1), Arg1 (Mm00475988_m1), Chil3 (Mm00657889_mH), Il33 (Mm00505403_m1), Icos (Mm00497600_m1), Il1O (Mm01288386_m1), Il1r/1 (Mm00516117_m1), Il17rb (Mm00444709_m1), Cr/f2 (Mm00497362_m1), and Actb (Applied Biosystems). Gene expression levels were quantified relative to Actb of the same sample.

Cytokine quantification: Cytokine concentrations were quantified using the LegendPlex Mouse Th cytokine and LegendPlex Mouse Inflammation Panel (BioLegend) per manufacturer's instructions and analyzed on an LSRII or LSRFortessa (BD Biosciences). ELISA for IL-33 was performed on lung supernatant according to the manufacturer's instructions (BioLegend).

Gene Expression Analysis: Nanostring gene expression analysis was performed using custom code sets per manufacturer's instructions (13). For RNA-seq, sorted alveolar macrophages were lysed with RLT Plus buffer and RNA was extracted using the RNeasy Plus Mini Kit (Qiagen). RNA was quantified using a Qubit RNA HS Assay kit (Invitrogen) and quality assessed with an RNA 6000 Pico Kit (Agilent). 2 ng of RNA were used as input for a modified SMART-Seq2 protocol (37) entailing RNA secondary structure denaturation (72° C. for three minutes), reverse transcription with Maxima Reverse Transcriptase (Life Technologies), and whole transcriptome amplification (WTA) with KAPA HiFi HotStart ReadyMix 2X (Kapa Biosystems) for 12 cycles. WTA products were purified with Ampure XP beads (Beckman Coulter), quantified with a Qubit dsDNA HS Assay Kit (Invitrogen), and quality accessed with a High Sensitivity DNA Chip run on a Bioanalyzer 2100 system (Agilent). 0.2 ng of purified WTA product was used as input for Nextera XT DNA Library Preparation Kit (Illumina). Uniquely barcoded libraries were pooled and sequenced with a NextSeq 500 sequencer using a high output V2 75 cycle kit (Illumina) and 2×38 paired end reads.

RNA-seq data processing: Raw data was converted to fastq files using bcl2fastq 2.17.1.14 with options “--minimum-trimmed-read-length 10--mask-short-adapter-reads 10”. Transcript quantification was done using Kallisto 0.42.3 (38) with the mm10 mouse genome annotation, and transcript counts were then converted to gene counts and normalized TPM values using the R package tximport (39).

RNA-seq analysis: Differential expression was analyzed using the DESeq2 R package (40). Gene expression was modeled as a linear function of genotype (alveolar macrophages). Significance of the overall model fit was estimated by a likelihood ratio test (LRT), where the reduced model consisted of only the intercept. P-values were automatically adjusted by DESeq2 using independent filtering for an FDR of 0.1. For alveolar macrophages, genes that had an LRT-adjusted p-value of at most 0.05, are included on the “Stats” tabs of Table S1, and genes were considered significantly differentially expressed for the purpose of downstream analysis and figures if they met the additional criteria of having a Wald-test adjusted p-value for the condition variable of at most 0.05, and absolute value of the associated log fold change of at least log 2(1.5) (“Rankings” tabs of Table S1).

Lung Histology: Lung lobe was fixed in 10% buffered formalin, embedded in paraffin, and processed for H&E and PAS staining. Sections were scored blindly by histopathologist to determine the severity of inflammation. Following scoring system was used: 0, normal; 1, mild; 2, moderate; 3, severe.

Methacholine challenge: To assess the airway hyperreactivity, methacholine challenge was performed on mice using a flexiVent rodent ventilator (SciReq) as described previously (41).

Statistics: With the exception of RNA-seq analysis, all statistical analysis was performed using GraphPad Prism 7. Data are shown as Mean±SEM unless otherwise indicated. Statistical significance was evaluated using Student's T-test, One-way, or Two-way ANOVA.

TABLE 1
Stats for all genes

| | padj.lrt | log2FC.CLEC2nn_vs_CLEC2pn | padj.CLEC2nn_vs_CLEC2pn | log2TPM.CLEC2nn | log2TPM.CLEC2pn |
|---|---|---|---|---|---|
| Plxnb2 | 5.8821E−20 | 1.172744681 | 3.01218E−21 | 6.085866507 | 6.407376688 |
| Sqle | 2.51682E−12 | 1.269233403 | 3.03696E−13 | 4.861040987 | 4.759998225 |
| Ccl6 | 6.76822E−12 | 0.526500891 | 2.55167E−12 | 12.54313105 | 12.12109999 |
| Gal | 1.29656E−10 | −0.761016414 | 1.55634E−10 | 5.913071227 | 6.73183557 |
| H2-Q9 | 2.94518E−10 | 2.877097129 | 1.08308E−12 | 6.919092888 | 5.417673868 |
| Tgtp1 | 1.03539E−09 | 9.355559677 | 1.40247E−08 | 3.654678657 | 1.762704573 |
| Cdh1 | 9.23434E−09 | 0.752447078 | 4.56903E−09 | 3.959164439 | 3.418015752 |
| H2-K1 | 1.81752E−08 | 0.580206586 | 1.04664E−08 | 10.99509758 | 10.63293614 |
| Clec1b | 5.08317E−08 | −4.251281949 | 3.84571E−09 | 0.695560394 | 3.678794768 |
| H2-M2 | 6.38414E−08 | 6.222224715 | 1.54468E−08 | 1.892541466 | 0.229101508 |
| Mgst3 | 2.73624E−07 | −0.829502883 | 2.943E−07 | 6.509599852 | 7.31306502 |
| Ccdc80 | 4.18823E−07 | 1.143671535 | 1.27245E−07 | 5.882414989 | 4.879022856 |
| C3 | 8.13984E−07 | 1.016528576 | 2.943E−07 | 7.168517437 | 6.728680618 |
| Cd300a | 9.66833E−07 | 0.794571219 | 4.7749E−07 | 7.101255719 | 7.197870305 |
| Slamf8 | 4.84723E−06 | 4.526822225 | 5.56444E−08 | 6.666417622 | 6.215801919 |
| Mt2 | 7.70627E−06 | 0.842318125 | 3.89674E−06 | 6.125877791 | 5.354554362 |
| Serpina3f | 7.70627E−06 | 9.411369079 | 4.84588E−07 | 2.919077917 | 1.328076388 |
| Cd52 | 8.708E−06 | 0.828565831 | 3.89674E−06 | 11.12232749 | 10.61670245 |
| Rilpl2 | 8.92314E−06 | 0.839639372 | 4.04753E−06 | 7.12003306 | 6.776062523 |
| Enpp5 | 9.9384E−06 | 2.794779652 | 4.8757E−07 | 2.792766854 | 2.546198424 |
| Rgs12 | 9.9384E−06 | 2.640659286 | 1.31709E−06 | 2.056300727 | 2.56269333 |
| Gstm1 | 1.72387E−05 | 0.369125925 | 1.10181E−05 | 8.489175064 | 8.458187167 |
| Pla2g7 | 2.58562E−05 | 2.841217775 | 2.10861E−06 | 8.244113715 | 7.845773453 |
| Gbp4 | 2.70485E−05 | 2.186321382 | 4.04753E−06 | 3.749038089 | 2.483740637 |
| Irf5 | 2.96394E−05 | 0.612744069 | 1.50322E−05 | 8.214626377 | 8.400678097 |
| Best1 | 3.14177E−05 | 0.897279328 | 1.40725E−05 | 4.232138357 | 3.493126957 |
| H2-Q4 | 3.34421E−05 | 1.517337803 | 1.10181E−05 | 4.730333181 | 4.027376727 |
| Tgfb2 | 6.29743E−05 | −2.359000026 | 3.48865E−05 | 0.616358285 | 1.938596382 |
| Ccl9 | 6.72996E−05 | 1.272153402 | 2.22348E−05 | 6.77222631 | 6.687953696 |
| Ccr5 | 6.72996E−05 | 1.70123058 | 1.40725E−05 | 5.067480612 | 3.867517789 |
| Igf1 | 6.72996E−05 | 2.240848141 | 9.7592E−06 | 2.534887456 | 1.18625991 |
| L1cam | 6.72996E−05 | −1.093588432 | 6.58053E−05 | 3.070219715 | 4.144942336 |
| Cxcl16 | 7.43458E−05 | 2.086765622 | 1.24413E−05 | 4.698515489 | 3.552055056 |
| C1qa | 9.39617E−05 | 6.494034162 | 0.000190574 | 5.300816482 | 2.924810002 |
| Trpm2 | 0.000106153 | 3.128369134 | 1.10181E−05 | 2.171642509 | 1.68852502 |
| Lyz2 | 0.000112188 | 0.504005388 | 7.33938E−05 | 15.77349565 | 15.72432421 |
| Cpne5 | 0.000115589 | −0.571455141 | 0.0001196 | 4.340322275 | 4.95225823 |
| Vcam1 | 0.000118105 | 4.262098294 | 3.89674E−06 | 2.111876728 | 2.371516812 |
| Fbp1 | 0.00014253 | 9.001064234 | 9.0263E−06 | 1.526269673 | 0.147659025 |
| Ch25h | 0.000144872 | 1.110350683 | 6.62932E−05 | 6.576704001 | 5.601902141 |
| Marco | 0.000237065 | 0.926102452 | 0.00012467 | 8.339672654 | 7.48040929 |
| AA467197 | 0.000269462 | 3.91416027 | 1.10181E−05 | 4.219442606 | 1.3822906 |
| Pla2g2d | 0.000281468 | 3.951256735 | 6.12637E−05 | 1.006340756 | 0.455267628 |
| Smpdl3b | 0.000297804 | 0.923675908 | 0.000169135 | 6.719685715 | 6.061992874 |
| H2-DMb1 | 0.000420066 | 0.696722875 | 0.000271252 | 9.122284779 | 8.924018704 |
| Gbp6 | 0.00043596 | 1.543412635 | 0.000158184 | 4.224854687 | 2.724920524 |
| Tgtp2 | 0.00043855 | 2.437381601 | 6.58053E−05 | 6.364587555 | 5.231737674 |
| Lyz1 | 0.000482119 | 0.478010831 | 0.000374105 | 11.43659574 | 11.42697317 |
| Manba | 0.000511703 | −0.440214089 | 0.000520643 | 5.092363385 | 5.534291987 |
| Ifi47 | 0.000529034 | 1.414700604 | 0.000222508 | 7.666963002 | 7.11845447 |
| Il1a | 0.0005521 | 0.748904184 | 0.000385688 | 5.6401404 | 5.024136434 |
| Mgst1 | 0.0005521 | 0.490935102 | 0.000439821 | 9.959719087 | 9.494501283 |
| 9130019O22Rik | 0.000648636 | −1.836464439 | 0.000611653 | 0.77985958 | 1.360018297 |
| Sc4mol | 0.000649181 | 0.807649267 | 0.000439821 | 6.659754948 | 6.684847485 |
| Cyp1a1 | 0.000765504 | 3.553124118 | 3.31165E−05 | 5.818659266 | 4.117922759 |
| Pilrb2 | 0.000825238 | 1.087123407 | 0.000479911 | 5.941392168 | 5.534913945 |
| Plxna1 | 0.000883455 | 0.740563485 | 0.000611653 | 2.595666862 | 2.515632984 |
| Mthfd2 | 0.001019755 | −0.75696241 | 0.001061079 | 5.072827718 | 5.158703929 |
| Ifitm3 | 0.001126459 | 1.330331522 | 0.000579068 | 11.95259129 | 11.97735132 |
| Itga4 | 0.001126459 | −0.705459207 | 0.00111102 | 5.141551781 | 5.560074973 |
| Pbx1 | 0.001169144 | 1.308547063 | 0.000611653 | 2.059263916 | 1.375027074 |
| Cd5l | 0.001201564 | 9.922505398 | 1.10181E−05 | 2.026167082 | 0 |
| Mt1 | 0.001339187 | 0.409273491 | 0.001094479 | 9.647721487 | 9.259263972 |
| Ptpro | 0.001395294 | 0.923441752 | 0.000962245 | 6.329712263 | 6.283342559 |
| Car4 | 0.001431122 | 0.54863901 | 0.00111102 | 10.05841231 | 9.73703181 |
| Csf1r | 0.001431122 | 0.53315124 | 0.00111102 | 9.391990257 | 9.486431144 |
| Ctla4 | 0.001431122 | 7.048300412 | 0.000873071 | 1.693190136 | 1.281829583 |
| Fam214a | 0.001431122 | −1.128276402 | 0.00143629 | 1.656095489 | 2.253562364 |
| Ptgs1 | 0.001431122 | 0.69245175 | 0.001104556 | 4.010882198 | 3.423367735 |
| Serpinb1a | 0.001431122 | −0.405826024 | 0.00143629 | 6.823262426 | 7.320564555 |
| Mmab | 0.001436731 | 1.176542774 | 0.000975994 | 2.234624516 | 1.970764097 |
| Cyp27a1 | 0.001467908 | 1.559112306 | 0.000751412 | 5.409354699 | 5.582952198 |
| H2-Q8 | 0.001675737 | 2.168529466 | 0.000520643 | 5.269573214 | 4.316857982 |
| Acaa1b | 0.002000753 | −0.501565132 | 0.002070133 | 7.508409634 | 8.069679765 |
| Fdps | 0.002017228 | 0.638662568 | 0.001489876 | 6.774760462 | 6.897148358 |
| Fads1 | 0.002023401 | 0.912120647 | 0.00143922 | 2.570311428 | 2.659272307 |
| Itgad | 0.002061877 | −1.043056791 | 0.002212337 | 2.530950646 | 3.478410015 |
| Tsc22d3 | 0.002244276 | −0.865326481 | 0.002323374 | 7.659766041 | 8.386898785 |
| Gdf15 | 0.002451513 | 0.77116832 | 0.001736971 | 5.900184101 | 5.219708252 |
| Cxcr1 | 0.002829037 | 1.364527216 | 0.00150471 | 3.435721426 | 2.299394137 |
| Alppl2 | 0.00290297 | −7.589073799 | 0.001032679 | 0 | 0.577082016 |
| Sfxn3 | 0.002955055 | −0.415303896 | 0.00289271 | 5.965549536 | 6.521543639 |
| Wfdc17 | 0.002955055 | 1.446639177 | 0.00143922 | 8.313903936 | 7.114425331 |
| Cyp51 | 0.003298329 | 0.827945324 | 0.002268515 | 5.032385209 | 5.302885025 |
| Socs3 | 0.003402776 | 0.94639708 | 0.002268515 | 7.039610823 | 7.382990644 |
| Aldh18a1 | 0.003817536 | −0.500435052 | 0.003760027 | 4.072872843 | 4.34515102 |
| Hmox1 | 0.003817536 | 0.72940396 | 0.002667771 | 7.709027793 | 7.331508172 |
| Plac8 | 0.003864203 | 2.601280275 | 0.00111102 | 12.39045293 | 11.25866154 |
| Ciita | 0.003918686 | 1.443288882 | 0.002233757 | 4.79077525 | 4.317393727 |
| Fth1 | 0.003920645 | 0.338391996 | 0.003264119 | 13.72595199 | 13.43694681 |
| Gm12250 | 0.00478707 | 2.567438065 | 0.001252861 | 4.248786895 | 3.559828749 |
| Earl1 | 0.004983811 | 3.391341838 | 0.00111102 | 2.627138878 | 0.547386355 |
| Tspan32 | 0.004983811 | 0.588358219 | 0.003760027 | 5.837127838 | 5.412445252 |
| Sema4d | 0.005427333 | 2.555129403 | 0.001336011 | 6.600307261 | 6.767969901 |
| Syngr1 | 0.005479265 | 3.09714705 | 0.002323374 | 1.70549538 | 0.380157761 |
| Eps8 | 0.005565023 | −3.300087752 | 0.002667771 | 1.174269916 | 3.667945547 |
| Neurl3 | 0.005751872 | 1.139866767 | 0.003717957 | 4.7612489 | 4.567188766 |
| Lss | 0.005980038 | 0.900478121 | 0.004130619 | 3.377531749 | 3.072955814 |
| Gapvd1 | 0.006254328 | −0.477200354 | 0.006368403 | 3.804188341 | 4.255926807 |
| Tbc1d8 | 0.006254328 | 2.35152487 | 0.002635661 | 4.958158294 | 4.877484075 |
| Bpifb1 | 0.00676001 | 7.010226334 | 0.002323374 | 0.489248895 | 0 |
| Fdft1 | 0.006841679 | 0.592949521 | 0.005560684 | 4.773598817 | 4.804925199 |
| Heatr5a | 0.007040101 | −0.737759849 | 0.007332952 | 2.472230403 | 3.211070617 |
| Igtp | 0.007040101 | 0.952656046 | 0.004948027 | 6.182828586 | 5.496083602 |
| Acp5 | 0.007151207 | 0.46534802 | 0.006307486 | 8.004748932 | 7.562024006 |
| Dgat1 | 0.007151207 | 0.624172919 | 0.006041918 | 5.089090789 | 5.022088865 |
| Lgmn | 0.007151207 | 0.489642579 | 0.006183028 | 8.006279087 | 7.817017744 |
| Mlph | 0.007151207 | −1.007342073 | 0.007464004 | 1.799178524 | 2.585138659 |
| Pros1 | 0.007151207 | −0.476760526 | 0.007445486 | 7.048777155 | 7.578277704 |
| Sepp1 | 0.007151207 | −0.336367198 | 0.007445486 | 10.10614766 | 10.5306811 |
| Hebp1 | 0.007318047 | 0.421362701 | 0.006483835 | 7.845705028 | 7.221548481 |
| Asrgl1 | 0.007322453 | 2.327766714 | 0.00300021 | 2.892262538 | 2.505378784 |
| Hsd3b7 | 0.007338155 | −0.440095573 | 0.007840324 | 7.773135346 | 8.237567489 |
| Flna | 0.00757054 | −0.503121299 | 0.008088638 | 7.456894426 | 8.153019103 |
| Aldoc | 0.00794044 | 0.451060856 | 0.007249723 | 5.759279361 | 5.400380354 |
| Ccrl2 | 0.00794044 | 0.751994393 | 0.006368403 | 7.666409544 | 7.162266944 |
| Zzef1 | 0.00818594 | −0.612571009 | 0.008506509 | 2.255433884 | 2.701992923 |
| Xpc | 0.008308855 | −0.786919415 | 0.008738748 | 3.39951412 | 4.155815333 |
| Cd68 | 0.008390103 | 0.306852055 | 0.00784287 | 10.26267877 | 10.22585271 |
| Mvd | 0.008390103 | 0.524275557 | 0.007445486 | 5.961328138 | 5.849689494 |
| Spint1 | 0.008847043 | 1.20374688 | 0.006041918 | 4.803647137 | 3.516836329 |
| Gltp | 0.00906412 | 0.352562516 | 0.008250988 | 7.691234038 | 7.644595382 |
| Tor3a | 0.009345546 | 0.493322624 | 0.008243014 | 5.658487497 | 5.544517843 |
| Aoah | 0.010215191 | 4.88095814 | 0.002212337 | 3.894937517 | 3.31149343 |
| Ly6i | 0.010437891 | 4.231951819 | 0.003062119 | 7.840690651 | 6.47890086 |
| Acer3 | 0.010627378 | 0.771738626 | 0.008490501 | 5.997168837 | 5.954296481 |
| Cspg4 | 0.010627378 | −1.733892777 | 0.010100125 | 0.372967277 | 1.049737282 |
| Irf8 | 0.010627378 | 0.481202601 | 0.009083 | 7.031423732 | 7.097276664 |
| Beta-s | 0.010724414 | 2.150555854 | 0.004324571 | 6.466260038 | 4.867734811 |
| Ngfrap1 | 0.010724414 | 1.17165424 | 0.008192939 | 3.94665201 | 3.746299328 |
| Cidec | 0.010871937 | −0.56240369 | 0.011417214 | 7.659624144 | 8.28145894 |
| Rasa3 | 0.010979754 | 0.691825529 | 0.008976468 | 6.230070887 | 6.503006256 |
| Napsa | 0.011168877 | 0.583150129 | 0.009424052 | 8.9238515 | 8.903542768 |
| Ccnb2 | 0.011231519 | 0.845204061 | 0.008795544 | 4.692168192 | 3.974086127 |
| Mmp12 | 0.012096541 | 3.05863161 | 0.002445411 | 3.99463253 | 1.711840451 |
| Gpr35 | 0.012517875 | 0.837663624 | 0.009603115 | 5.79508897 | 5.767173656 |
| Tcirg1 | 0.012660657 | 0.345558197 | 0.011417214 | 6.51919581 | 6.319213136 |
| D8Ertd82e | 0.013016543 | −0.509416616 | 0.013513805 | 2.976848904 | 3.493701767 |
| AU021092 | 0.013701873 | 5.968611399 | 0.012404195 | 1.322325113 | 0.928998813 |
| Cyfip2 | 0.014491599 | 1.591582206 | 0.008471778 | 4.52769525 | 4.587194703 |
| Lrrc25 | 0.014491599 | 0.827833467 | 0.011465557 | 6.814628606 | 6.830124309 |
| Cdkn1b | 0.014791887 | −0.954568503 | 0.01549284 | 3.506753146 | 4.259441859 |
| Clec4n | 0.014791887 | 0.525765489 | 0.012538171 | 9.660807783 | 9.156917381 |
| Prkcd | 0.014791887 | 0.577194949 | 0.012400712 | 8.519171331 | 8.57186171 |
| Col6a1 | 0.014818033 | −1.644892658 | 0.015328366 | 1.312708587 | 2.087521679 |
| Egfem1 | 0.015026339 | −0.795785847 | 0.015935663 | 2.648408002 | 3.39507521 |
| Sc5d | 0.017160438 | 0.992360055 | 0.012404195 | 4.755355634 | 4.821396356 |
| Irf1 | 0.018025021 | 0.634789994 | 0.014988106 | 7.341344897 | 7.10531853 |
| Gss | 0.018276651 | 0.677387647 | 0.015328366 | 4.739725263 | 4.427769629 |
| Tnfrsf1b | 0.018795061 | 0.315883228 | 0.016696285 | 7.951294916 | 8.167898128 |
| Ppp1r10 | 0.01895444 | −0.4760713 | 0.018971884 | 4.427044606 | 5.007567791 |
| Fabp5 | 0.01923614 | 0.68928524 | 0.01549284 | 8.298990466 | 7.624159349 |
| Gtf2h1 | 0.020121746 | 0.437470223 | 0.017266041 | 4.824973374 | 4.794557963 |
| Lamc2 | 0.020185839 | −7.076294271 | 0.009424052 | 0.299629901 | 1.135783714 |
| Rnd3 | 0.02021698 | −0.450154801 | 0.020329613 | 5.158757611 | 5.744190703 |
| Slc16a1 | 0.020914504 | 0.945910644 | 0.01604141 | 2.025363509 | 1.651110795 |
| Cxcl14 | 0.021037385 | 1.261768309 | 0.014988106 | 2.269934624 | 1.825571276 |
| Wdr13 | 0.021562717 | −0.654063703 | 0.021256423 | 2.638129458 | 3.262169782 |
| Lgals1 | 0.021667438 | −0.25344949 | 0.021149378 | 8.95901771 | 9.119504461 |
| Lmna | 0.021667438 | −0.502815983 | 0.021393748 | 7.817771157 | 8.348856225 |
| Idi1 | 0.021726555 | 0.843208492 | 0.016922931 | 5.303445146 | 5.377521165 |
| Slc11a1 | 0.025176498 | 2.906996333 | 0.008421446 | 6.85434708 | 6.751799091 |
| Trem2 | 0.025365837 | 0.695223393 | 0.020329613 | 5.660900352 | 5.316691357 |
| Cars | 0.025371534 | −0.500032053 | 0.024098784 | 3.589850073 | 3.971211769 |
| Gp49a | 0.025476727 | 0.55678177 | 0.021102593 | 6.912902656 | 6.476098651 |
| Fgl2 | 0.025831606 | 1.307223501 | 0.017266041 | 6.459496149 | 5.913040302 |
| Slc22a4 | 0.025831606 | 5.764706557 | 0.021256423 | 1.62699438 | 1.527463769 |
| Flt3 | 0.026307065 | 6.525107165 | 0.011417214 | 1.536804274 | 2.774238863 |
| Limd2 | 0.026307065 | 1.354027094 | 0.017683869 | 6.6018147 | 6.588379317 |
| Plec | 0.028807803 | −0.532787176 | 0.027587726 | 3.191854334 | 4.017225149 |
| Ccl17 | 0.030191961 | 2.557829156 | 0.013537463 | 3.038424504 | 1.091491101 |
| Ccl4 | 0.030391012 | 0.849900969 | 0.022654901 | 6.971016817 | 6.460615618 |
| Clec4a3 | 0.031043274 | −0.320654376 | 0.029451626 | 10.09034006 | 10.30435608 |
| Ptgir | 0.031043274 | 1.206172754 | 0.020938451 | 3.841155587 | 3.749408764 |
| AB124611 | 0.031145583 | 1.137516456 | 0.022165066 | 7.569417394 | 7.436400422 |
| Fam122b | 0.031171736 | −0.47435516 | 0.030010379 | 2.596988914 | 2.925720471 |
| H2-Ab1 | 0.031712976 | 2.72099117 | 0.009871769 | 12.55074159 | 12.2789162 |
| C1rl | 0.031915418 | 0.732134296 | 0.024841311 | 3.831345504 | 3.004501692 |
| Ly6c1 | 0.032012499 | 1.481600024 | 0.019738105 | 7.408113013 | 7.413020191 |
| Ift80 | 0.032217836 | 2.545864661 | 0.016330947 | 0.77989109 | 0.1490053 |
| Slc48a1 | 0.032927072 | 0.329650995 | 0.028442267 | 7.057094564 | 7.015481958 |
| Rtf1 | 0.034141735 | 0.958717726 | 0.025600538 | 1.739411773 | 1.425750214 |
| Mmp14 | 0.034548834 | 6.877916886 | 0.010956964 | 2.05382631 | 1.120907388 |
| Card11 | 0.035061439 | 0.268149939 | 0.031230199 | 6.009265921 | 5.825223905 |
| Egflam | 0.035061439 | 1.520325637 | 0.023210948 | 1.554526108 | 2.407893921 |
| Gbp2 | 0.035061439 | 0.861966465 | 0.026373021 | 7.85862136 | 6.485763309 |
| Sycp2 | 0.035061439 | 5.822066282 | 0.023804639 | 0.148581211 | 0.002414037 |
| Adcy3 | 0.036715537 | −0.326038402 | 0.035175863 | 5.680527786 | 6.068376383 |
| Syne3 | 0.036715537 | −1.452401801 | 0.035175863 | 1.195496275 | 2.256504664 |
| Cldn7 | 0.037921843 | 3.106336783 | 0.018971884 | 1.260839194 | 0.530709107 |
| Csgalnact1 | 0.037921843 | 3.628922924 | 0.011417214 | 1.344074648 | 1.588328866 |
| Cxcl3 | 0.037921843 | 1.777951281 | 0.021256423 | 3.326318436 | 1.863980793 |
| Ifitm2 | 0.037921843 | 0.543228207 | 0.032322742 | 10.36942681 | 10.38905425 |
| Pafah1b1 | 0.037921843 | −1.017584758 | 0.036990015 | 2.422643411 | 2.924949603 |
| Pitpnm1 | 0.037921843 | −0.491360406 | 0.036365904 | 4.556561071 | 4.962614316 |
| 0610007P14Rik | 0.038730613 | 0.638315033 | 0.03266162 | 4.981772184 | 4.905476864 |
| C1qc | 0.03885851 | 4.780668946 | 0.021908781 | 5.139687885 | 2.59626459 |
| Ano1 | 0.039074142 | −6.045242105 | 0.025342072 | 0.068104178 | 0.754436491 |
| Pvrl2 | 0.039074142 | 1.883913033 | 0.021908781 | 2.230408933 | 1.528085171 |
| Rplp1 | 0.03908237 | −0.218539 | 0.037619098 | 11.52871293 | 11.45621407 |
| Tmem43 | 0.039139529 | −0.501851623 | 0.038761776 | 6.055321757 | 6.712377278 |
| Ctsd | 0.040319785 | 0.203932484 | 0.036405375 | 12.67931781 | 12.53936273 |
| Prdx1 | 0.040319785 | 0.237737258 | 0.036365904 | 10.82849057 | 10.66875146 |
| B2m | 0.041672799 | 0.376292689 | 0.036365904 | 13.04322107 | 12.80400461 |
| Srxn1 | 0.042066875 | 0.816405619 | 0.034579256 | 2.695286207 | 2.248774759 |
| Cxcl10 | 0.042731801 | 2.781948666 | 0.017266041 | 5.013212837 | 4.074126531 |
| Vrk1 | 0.042731801 | −0.280369219 | 0.041296071 | 5.486090697 | 5.777813166 |
| Ncam2 | 0.0434267 | −3.063358989 | 0.032258292 | 0.145780413 | 0.838352479 |
| Abcc8 | 0.043621504 | 7.41048142 | 0.009424052 | 0.217938394 | 0 |
| Ear12 | 0.043719247 | 0.367814491 | 0.038761776 | 8.558058224 | 8.324032969 |
| Ear3 | 0.043719247 | 0.367814491 | 0.038761776 | 8.558058224 | 8.324032969 |
| Ncoa2 | 0.044357878 | −0.500893002 | 0.043471278 | 2.726545218 | 3.15912598 |
| Asns | 0.045922982 | −1.289408387 | 0.045023615 | 1.882129652 | 2.822348437 |
| Nek2 | 0.045922982 | 1.013768523 | 0.035865218 | 1.891410194 | 1.33463098 |
| Trp53i11 | 0.045922982 | −0.955848751 | 0.045480309 | 2.746720684 | 3.530976929 |
| Alas1 | 0.046030679 | 0.299352836 | 0.041296071 | 6.802253166 | 6.604839084 |
| Agmo | 0.046540359 | −0.543939153 | 0.045480309 | 4.627547965 | 5.215775977 |
| Ldlr | 0.048838522 | 0.655783026 | 0.040423575 | 6.254890093 | 6.648526997 |
| Aldh2 | 0.048860469 | 0.365039039 | 0.043223646 | 7.235883075 | 7.416957091 |
| Rps12 | 0.04909024 | −0.278509416 | 0.046688616 | 10.40943297 | 10.15276862 |
| Fam20c | 0.049119782 | 3.145692644 | 0.017865832 | 3.382980949 | 2.899913421 |
| Map4k1 | 0.049119782 | 0.640814704 | 0.041296071 | 5.278162285 | 5.084140112 |
| Ptgs2 | 0.049119782 | 3.005825577 | 0.023210948 | 4.582627233 | 4.718632173 |

TABLE 2
Ranked Candidates

| | padj.lrt | log2FC.CLEC2nn_vs_CLEC2pn | padj.CLEC2nn_vs_CLEC2pn | rank.padj.lrt |
|---|---|---|---|---|
| Plxnb2 | 5.8821E−20 | 1.172744681 | 3.01218E−21 | 1 |
| Sqle | 2.51682E−12 | 1.269233403 | 3.03696E−13 | 2 |
| Gal | 1.29656E−10 | −0.761016414 | 1.55634E−10 | 4 |
| H2-Q9 | 2.94518E−10 | 2.877097129 | 1.08308E−12 | 5 |
| Tgtp1 | 1.03539E−09 | 9.355559677 | 1.40247E−08 | 6 |
| Cdh1 | 9.23434E−09 | 0.752447078 | 4.56903E−09 | 7 |
| Clec1b | 5.08317E−08 | −4.251281949 | 3.84571E−09 | 9 |
| H2-M2 | 6.38414E−08 | 6.222224715 | 1.54468E−08 | 10 |
| Mgst3 | 2.73624E−07 | −0.829502883 | 2.943E−07 | 11 |
| Ccdc80 | 4.18823E−07 | 1.143671535 | 1.27245E−07 | 12 |
| C3 | 8.13984E−07 | 1.016528576 | 2.943E−07 | 13 |
| Cd300a | 9.66833E−07 | 0.794571219 | 4.7749E−07 | 14 |
| Slamf8 | 4.84723E−06 | 4.526822225 | 5.56444E−08 | 15 |
| Mt2 | 7.70627E−06 | 0.842318125 | 3.89674E−06 | 16.5 |
| Serpina3f | 7.70627E−06 | 9.411369079 | 4.84588E−07 | 16.5 |
| Cd52 | 8.708E−06 | 0.828565831 | 3.89674E−06 | 18 |
| Rilpl2 | 8.92314E−06 | 0.839639372 | 4.04753E−06 | 19 |
| Enpp5 | 9.9384E−06 | 2.794779652 | 4.8757E−07 | 20.5 |
| Rgs12 | 9.9384E−06 | 2.640659286 | 1.31709E−06 | 20.5 |
| Pla2g7 | 2.58562E−05 | 2.841217775 | 2.10861E−06 | 23 |
| Gbp4 | 2.70485E−05 | 2.186321382 | 4.04753E−06 | 24 |
| Irf5 | 2.96394E−05 | 0.612744069 | 1.50322E−05 | 25 |
| Best1 | 3.14177E−05 | 0.897279328 | 1.40725E−05 | 26 |
| H2-Q4 | 3.34421E−05 | 1.517337803 | 1.10181E−05 | 27 |
| Tgfb2 | 6.29743E−05 | −2.359000026 | 3.48865E−05 | 28 |
| Ccl9 | 6.72996E−05 | 1.272153402 | 2.22348E−05 | 30.5 |
| Ccr5 | 6.72996E−05 | 1.70123058 | 1.40725E−05 | 30.5 |
| Igf1 | 6.72996E−05 | 2.240848141 | 9.7592E−06 | 30.5 |
| L1cam | 6.72996E−05 | −1.093588432 | 6.58053E−05 | 30.5 |
| Cxcl16 | 7.43458E−05 | 2.086765622 | 1.24413E−05 | 33 |
| C1qa | 9.39617E−05 | 6.494034162 | 0.000190574 | 34 |
| Trpm2 | 0.000106153 | 3.128369134 | 1.10181E−05 | 35 |
| Vcam1 | 0.000118105 | 4.262098294 | 3.89674E−06 | 38 |
| Fbp1 | 0.00014253 | 9.001064234 | 9.0263E−06 | 39 |
| Ch25h | 0.000144872 | 1.110350683 | 6.62932E−05 | 40 |
| Marco | 0.000237065 | 0.926102452 | 0.00012467 | 41 |
| AA467197 | 0.000269462 | 3.91416027 | 1.10181E−05 | 42 |
| Pla2g2d | 0.000281468 | 3.951256735 | 6.12637E−05 | 43 |
| Smpdl3b | 0.000297804 | 0.923675908 | 0.000169135 | 44 |
| H2-DMb1 | 0.000420066 | 0.696722875 | 0.000271252 | 45 |
| Gbp6 | 0.00043596 | 1.543412635 | 0.000158184 | 46 |
| Tgtp2 | 0.00043855 | 2.437381601 | 6.58053E−05 | 47 |
| Ifi47 | 0.000529034 | 1.414700604 | 0.000222508 | 50 |
| Il1a | 0.0005521 | 0.748904184 | 0.000385688 | 51.5 |
| 9130019O22Rik | 0.000648636 | −1.836464439 | 0.000611653 | 53 |
| Sc4mol | 0.000649181 | 0.807649267 | 0.000439821 | 54 |
| Cyp1a1 | 0.000765504 | 3.553124118 | 3.31165E−05 | 55 |
| Pilrb2 | 0.000825238 | 1.087123407 | 0.000479911 | 56 |
| Plxna1 | 0.000883455 | 0.740563485 | 0.000611653 | 57 |
| Mthfd2 | 0.001019755 | −0.75696241 | 0.001061079 | 58 |
| Ifitm3 | 0.001126459 | 1.330331522 | 0.000579068 | 59.5 |
| Itga4 | 0.001126459 | −0.705459207 | 0.00111102 | 59.5 |
| Pbx1 | 0.001169144 | 1.308547063 | 0.000611653 | 61 |
| Cd5l | 0.001201564 | 9.922505398 | 1.10181E−05 | 62 |
| Ptpro | 0.001395294 | 0.923441752 | 0.000962245 | 64 |
| Ctla4 | 0.001431122 | 7.048300412 | 0.000873071 | 67.5 |
| Fam214a | 0.001431122 | −1.128276402 | 0.00143629 | 67.5 |
| Ptgs1 | 0.001431122 | 0.69245175 | 0.001104556 | 67.5 |
| Mmab | 0.001436731 | 1.176542774 | 0.000975994 | 71 |
| Cyp27a1 | 0.001467908 | 1.559112306 | 0.000751412 | 72 |
| H2-Q8 | 0.001675737 | 2.168529466 | 0.000520643 | 73 |
| Fdps | 0.002017228 | 0.638662568 | 0.001489876 | 75 |
| Fads1 | 0.002023401 | 0.912120647 | 0.00143922 | 76 |
| Itgad | 0.002061877 | −1.043056791 | 0.002212337 | 77 |
| Tsc22d3 | 0.002244276 | −0.865326481 | 0.002323374 | 78 |
| Gdf15 | 0.002451513 | 0.77116832 | 0.001736971 | 79 |
| Cxcr1 | 0.002829037 | 1.364527216 | 0.00150471 | 80 |
| Alppl2 | 0.00290297 | −7.589073799 | 0.001032679 | 81 |
| Wfdc17 | 0.002955055 | 1.446639177 | 0.00143922 | 82.5 |
| Cyp51 | 0.003298329 | 0.827945324 | 0.002268515 | 84 |
| Socs3 | 0.003402776 | 0.94639708 | 0.002268515 | 85 |
| Hmox1 | 0.003817536 | 0.72940396 | 0.002667771 | 86.5 |
| Plac8 | 0.003864203 | 2.601280275 | 0.00111102 | 88 |
| Ciita | 0.003918686 | 1.443288882 | 0.002233757 | 89 |
| Gm12250 | 0.00478707 | 2.567438065 | 0.001252861 | 91 |
| Ear11 | 0.004983811 | 3.391341838 | 0.00111102 | 92.5 |
| Tspan32 | 0.004983811 | 0.588358219 | 0.003760027 | 92.5 |
| Sema4d | 0.005427333 | 2.555129403 | 0.001336011 | 94 |
| Syngr1 | 0.005479265 | 3.09714705 | 0.002323374 | 95 |
| Eps8 | 0.005565023 | −3.300087752 | 0.002667771 | 96 |
| Neurl3 | 0.005751872 | 1.139866767 | 0.003717957 | 97 |
| Lss | 0.005980038 | 0.900478121 | 0.004130619 | 98 |
| Tbc1d8 | 0.006254328 | 2.35152487 | 0.002635661 | 99.5 |
| Bpifb1 | 0.00676001 | 7.010226334 | 0.002323374 | 101 |
| Fdft1 | 0.006841679 | 0.592949521 | 0.005560684 | 102 |
| Heatr5a | 0.007040101 | −0.737759849 | 0.007332952 | 103.5 |
| Igtp | 0.007040101 | 0.952656046 | 0.004948027 | 103.5 |
| Dgat1 | 0.007151207 | 0.624172919 | 0.006041918 | 107.5 |
| Mlph | 0.007151207 | −1.007342073 | 0.007464004 | 107.5 |
| Asrgl1 | 0.007322453 | 2.327766714 | 0.00300021 | 112 |
| Ccrl2 | 0.00794044 | 0.751994393 | 0.006368403 | 115.5 |
| Zzef1 | 0.00818594 | −0.612571009 | 0.008506509 | 117 |
| Xpc | 0.008308855 | −0.786919415 | 0.008738748 | 118 |
| Spint1 | 0.008847043 | 1.20374688 | 0.006041918 | 121 |
| Aoah | 0.010215191 | 4.88095814 | 0.002212337 | 124 |
| Ly6i | 0.010437891 | 4.231951819 | 0.003062119 | 125 |
| Acer3 | 0.010627378 | 0.771738626 | 0.008490501 | 127 |
| Cspg4 | 0.010627378 | −1.733892777 | 0.010100125 | 127 |
| Beta-s | 0.010724414 | 2.150555854 | 0.004324571 | 129.5 |
| Ngfrap1 | 0.010724414 | 1.17165424 | 0.008192939 | 129.5 |
| Rasa3 | 0.010979754 | 0.691825529 | 0.008976468 | 132 |
| Ccnb2 | 0.011231519 | 0.845204061 | 0.008795544 | 134 |
| Mmp12 | 0.012096541 | 3.05863161 | 0.002445411 | 135 |
| Gpr35 | 0.012517875 | 0.837663624 | 0.009603115 | 136 |
| AU021092 | 0.013701873 | 5.968611399 | 0.012404195 | 139 |
| Cyfip2 | 0.014491599 | 1.591582206 | 0.008471778 | 140.5 |
| Lrrc25 | 0.014491599 | 0.827833467 | 0.011465557 | 140.5 |
| Cdkn1b | 0.014791887 | −0.954568503 | 0.01549284 | 143 |
| Col6a1 | 0.014818033 | −1.644892658 | 0.015328366 | 145 |
| Egfem1 | 0.015026339 | −0.795785847 | 0.015935663 | 146 |
| Sc5d | 0.017160438 | 0.992360055 | 0.012404195 | 147 |
| Irf1 | 0.018025021 | 0.634789994 | 0.014988106 | 148 |
| Gss | 0.018276651 | 0.677387647 | 0.015328366 | 149 |
| Fabp5 | 0.01923614 | 0.68928524 | 0.01549284 | 152 |
| Lamc2 | 0.020185839 | −7.076294271 | 0.009424052 | 154 |
| Slc16a1 | 0.020914504 | 0.945910644 | 0.01604141 | 156 |
| Cxcl14 | 0.021037385 | 1.261768309 | 0.014988106 | 157 |
| Wdr13 | 0.021562717 | −0.654063703 | 0.021256423 | 158 |
| Idi1 | 0.021726555 | 0.843208492 | 0.016922931 | 161 |
| Slc11a1 | 0.025176498 | 2.906996333 | 0.008421446 | 162 |
| Trem2 | 0.025365837 | 0.695223393 | 0.020329613 | 163 |
| Fgl2 | 0.025831606 | 1.307223501 | 0.017266041 | 166.5 |
| Slc22a4 | 0.025831606 | 5.764706557 | 0.021256423 | 166.5 |
| Flt3 | 0.026307065 | 6.525107165 | 0.011417214 | 168.5 |
| Limd2 | 0.026307065 | 1.354027094 | 0.017683869 | 168.5 |
| Ccl17 | 0.030191961 | 2.557829156 | 0.013537463 | 171 |
| Ccl4 | 0.030391012 | 0.849900969 | 0.022654901 | 172 |
| Ptgir | 0.031043274 | 1.206172754 | 0.020938451 | 173.5 |
| AB124611 | 0.031145583 | 1.137516456 | 0.022165066 | 175 |
| H2-Ab1 | 0.031712976 | 2.72099117 | 0.009871769 | 177 |
| C1rl | 0.031915418 | 0.732134296 | 0.024841311 | 178 |
| Ly6c1 | 0.032012499 | 1.481600024 | 0.019738105 | 179 |
| Ift80 | 0.032217836 | 2.545864661 | 0.016330947 | 180 |
| Rtf1 | 0.034141735 | 0.958717726 | 0.025600538 | 182 |
| Mmp14 | 0.034548834 | 6.877916886 | 0.010956964 | 183 |
| Egflam | 0.035061439 | 1.520325637 | 0.023210948 | 185.5 |
| Gbp2 | 0.035061439 | 0.861966465 | 0.026373021 | 185.5 |
| Sycp2 | 0.035061439 | 5.822066282 | 0.023804639 | 185.5 |
| Syne3 | 0.036715537 | −1.452401801 | 0.035175863 | 188.5 |
| Cldn7 | 0.037921843 | 3.106336783 | 0.018971884 | 192.5 |
| Csgalnact1 | 0.037921843 | 3.628922924 | 0.011417214 | 192.5 |
| Cxcl3 | 0.037921843 | 1.777951281 | 0.021256423 | 192.5 |
| Pafah1b1 | 0.037921843 | −1.017584758 | 0.036990015 | 192.5 |
| 0610007P14Rik | 0.038730613 | 0.638315033 | 0.03266162 | 196 |
| C1qc | 0.03885851 | 4.780668946 | 0.021908781 | 197 |
| Ano1 | 0.039074142 | −6.045242105 | 0.025342072 | 198.5 |
| Pvrl2 | 0.039074142 | 1.883913033 | 0.021908781 | 198.5 |
| Srxn1 | 0.042066875 | 0.816405619 | 0.034579256 | 205 |
| Cxcl10 | 0.042731801 | 2.781948666 | 0.017266041 | 206.5 |
| Ncam2 | 0.0434267 | −3.063358989 | 0.032258292 | 208 |
| Abcc8 | 0.043621504 | 7.41048142 | 0.009424052 | 209 |
| Asns | 0.045922982 | −1.289408387 | 0.045023615 | 214 |
| Nek2 | 0.045922982 | 1.013768523 | 0.035865218 | 214 |
| Trp53i11 | 0.045922982 | −0.955848751 | 0.045480309 | 214 |
| Ldlr | 0.048838522 | 0.655783026 | 0.040423575 | 218 |
| Fam20c | 0.049119782 | 3.145692644 | 0.017865832 | 222 |
| Map4k1 | 0.049119782 | 0.640814704 | 0.041296071 | 222 |
| Ptgs2 | 0.049119782 | 3.005825577 | 0.023210948 | 222 |

| | rank.log2FC.CLEC2nn_vs_CLEC2pn | rank.padj.CLEC2nn_vs_CLEC2pn |
|---|---|---|
| Plxnb2 | 86 | 1 |
| Sqle | 81 | 2 |
| Gal | 38 | 3 |
| H2-Q9 | 3 | 7 |
| Tgtp1 | 134 | 6 |
| Cdh1 | 22 | 5 |
| Clec1b | 121 | 11.5 |
| H2-M2 | 88 | 10 |
| Mgst3 | 97 | 11.5 |
| Ccdc80 | 128 | 13 |
| C3 | 20 | 9 |
| Cd300a | 118 | 19 |
| Slamf8 | 2 | 14 |
| Mt2 | 122 | 19 |
| Serpina3f | 119 | 21.5 |
| Cd52 | 40 | 15 |
| Rilpl2 | 43 | 16 |
| Enpp5 | 39 | 17 |
| Rgs12 | 54 | 21.5 |
| Pla2g7 | 112 | 30.5 |
| Gbp4 | 68 | 26.5 |
| Irf5 | 50 | 35 |
| Best1 | 80 | 33 |
| H2-Q4 | 62 | 30.5 |
| Tgfb2 | 53 | 24 |
| Ccl9 | 93 | 37.5 |
| Ccr5 | 57 | 29 |
| Igf1 | 12 | 43 |
| L1cam | 31 | 26.5 |
| Cxcl16 | 21 | 19 |
| C1qa | 4 | 23 |
| Trpm2 | 92 | 39 |
| Vcam1 | 24 | 36 |
| Fbp1 | 108 | 42 |
| Ch25h | 142 | 45 |
| Marco | 66 | 41 |
| AA467197 | 49 | 37.5 |
| Pla2g2d | 73 | 44 |
| Smpdl3b | 136 | 46 |
| H2-DMb1 | 59 | 52 |
| Gbp6 | 126 | 47 |
| Tgtp2 | 27 | 34 |
| Ifi47 | 133 | 59 |
| Il1a | 76 | 50 |
| 9130019O22Rik | 77 | 52 |
| Sc4mol | 1 | 26.5 |
| Cyp1a1 | 109 | 56 |
| Pilrb2 | 8 | 55 |
| Plxna1 | 91 | 66 |
| Mthfd2 | 144 | 60 |
| Ifitm3 | 85 | 57 |
| Itga4 | 65 | 54 |
| Pbx1 | 55 | 49 |
| Cd5l | 151 | 69 |
| Ptpro | 95 | 72.5 |
| Ctla4 | 74 | 70 |
| Fam214a | 5 | 58 |
| Ptgs1 | 71 | 67.5 |
| Mmab | 105 | 75.5 |
| Cyp27a1 | 140 | 82.5 |
| H2-Q8 | 44 | 62 |
| Fdps | 45 | 64 |
| Fads1 | 28 | 62 |
| Itgad | 158 | 87 |
| Tsc22d3 | 47 | 65 |
| Gdf15 | 33 | 78 |
| Cxcr1 | 29 | 82.5 |
| Alppl2 | 89 | 86 |
| Wfdc17 | 51 | 81 |
| Cyp51 | 9 | 78 |
| Socs3 | 157 | 91 |
| Hmox1 | 104 | 90 |
| Plac8 | 154 | 92.5 |
| Ciita | 99 | 96 |
| Gm12250 | 135 | 94 |
| Ear11 | 156 | 101 |
| Tspan32 | 129 | 102 |
| Sema4d | 84 | 92.5 |
| Syngr1 | 18 | 72.5 |
| Eps8 | 23 | 85 |
| Neurl3 | 130 | 100 |
| Lss | 61 | 109 |
| Tbc1d8 | 87 | 97 |
| Bpifb1 | 145 | 104 |
| Fdft1 | 116 | 103 |
| Heatr5a | 35 | 80 |
| Igtp | 120 | 107 |
| Dgat1 | 64 | 99 |
| Mlph | 103 | 121.5 |
| Asrgl1 | 153 | 117.5 |
| Ccrl2 | 106 | 124 |
| Zzef1 | 82 | 117.5 |
| Xpc | 149 | 136 |
| Spint1 | 143 | 133 |
| Aoah | 11 | 111.5 |
| Ly6i | 75 | 129 |
| Acer3 | 46 | 116 |
| Cspg4 | 115 | 141 |
| Beta-s | 90 | 140 |
| Ngfrap1 | 42 | 108 |
| Rasa3 | 69 | 132 |
| Ccnb2 | 101 | 147 |
| Mmp12 | 10 | 110 |
| Gpr35 | 67 | 142.5 |
| AU021092 | 70 | 152 |
| Cyfip2 | 32 | 131 |
| Lrrc25 | 26 | 111.5 |
| Cdkn1b | 60 | 136 |
| Col6a1 | 19 | 138.5 |
| Egfem1 | 14 | 146 |
| Sc5d | 58 | 138.5 |
| Irf1 | 125 | 151 |
| Gss | 41 | 127.5 |
| Fabp5 | 79 | 157 |
| Lamc2 | 102 | 158 |
| Slc16a1 | 30 | 130 |
| Cxcl14 | 150 | 156 |
| Wdr13 | 36 | 142.5 |
| Idi1 | 132 | 4 |
| Slc11a1 | 38 | 3 |
| Trem2 | 3 | 7 |
| Fgl2 | 13 | 8 |
| Slc22a4 | 121 | 11.5 |
| Flt3 | 88 | 10 |
| Limd2 | 97 | 11.5 |
| Ccl17 | 20 | 9 |
| Ccl4 | 118 | 19 |
| Ptgir | 122 | 19 |
| AB124611 | 119 | 21.5 |
| H2-Ab1 | 43 | 16 |
| C1rl | 39 | 17 |
| Ly6c1 | 54 | 21.5 |
| Ift80 | 155 | 32 |
| Rtf1 | 68 | 26.5 |
| Mmp14 | 50 | 35 |
| Egflam | 62 | 30.5 |
| Gbp2 | 53 | 24 |
| Sycp2 | 93 | 37.5 |
| Syne3 | 12 | 43 |
| Cldn7 | 31 | 26.5 |
| Csgalnact1 | 21 | 19 |
| Cxcl3 | 4 | 23 |
| Pafah1b1 | 107 | 40 |
| 0610007P14Rik | 24 | 36 |
| C1qc | 108 | 42 |
| Ano1 | 142 | 45 |
| Pvrl2 | 66 | 41 |
| Srxn1 | 27 | 34 |
| Cxcl10 | 94 | 48 |
| Ncam2 | 133 | 59 |
| Abcc8 | 76 | 50 |
| Asns | 109 | 56 |
| Nek2 | 8 | 55 |
| Trp53i11 | 91 | 66 |
| Ldlr | 65 | 54 |
| Fam20c | 110 | 67.5 |
| Map4k1 | 95 | 72.5 |
| Ptgs2 | 113 | 78 |

### REFERENCES

1. Hammad H, and Lambrecht B N. Barrier Epithelial Cells and the Control of Type 2 Immunity. Immunity. 2015; 43(1):29-40.
2. Van Dyken S J, Nussbaum J C, Lee J, Molofsky A B, Liang H E, Pollack J L, et al. A tissue checkpoint regulates type 2 immunity. Nat Immunol. 2016; 17(12):1381-7.
3. Corren J, Parnes J R, Wang L, Mo M, Roseti S L, Griffiths J M, et al. Tezepelumab in Adults with Uncontrolled Asthma. N Engl J Med. 2017; 377(10):936-46.
4. Israel E, and Reddel H K. Severe and Difficult-to-Treat Asthma in Adults. N Engl J Med. 2017; 377(10):965-76.
5. Snelgrove R J, Goulding J, Didierlaurent A M, Lyonga D, Vekaria S, Edwards L, et al. A critical function for CD200 in lung immune homeostasis and the severity of influenza infection. Nat Immunol. 2008; 9(9):1074-83.
6. Hussell T, and Bell T J. Alveolar macrophages: plasticity in a tissue-specific context. Nat Rev Immunol. 2014; 14(2):81-93.
7. Garbi N, and Lambrecht B N. Location, function, and ontogeny of pulmonary macrophages during the steady state. Pflugers Arch. 2017; 469(3-4):561-72.
8. Mathie S A, Dixon K L, Walker S A, Tyrrell V, Mondhe M, O'Donnell V B, et al. Alveolar macrophages are sentinels of murine pulmonary homeostasis following inhaled antigen challenge. Allergy. 2015; 70(1):80-9.
9. Purnama C, Ng S L, Tetlak P, Setiagani Y A, Kandasamy M, Baalasubramanian S, et al. Transient ablation of alveolar macrophages leads to massive pathology of influenza infection without affecting cellular adaptive immunity. Eur J Immunol. 2014; 44(7):2003-12.
10. Kuroda E, Ozasa K, Temizoz B, Ohata K, Koo C X, Kanuma T, et al. Inhaled Fine Particles Induce Alveolar Macrophage Death and Interleukin-1alpha Release to Promote Inducible Bronchus-Associated Lymphoid Tissue Formation. Immunity. 2016; 45(6):1299-310.
11. Yu X, Buttgereit A, Lelios I, Utz S G, Cansever D, Becher B, et al. The Cytokine TGF-beta Promotes the Development and Homeostasis of Alveolar Macrophages. Immunity. 2017; 47(5):903-12 e4.
12. Burastero S E, Magnani Z, Confetti C, Abbruzzese L, Oddera S, Balbo P, et al. Increased expression of the CD80 accessory molecule by alveolar macrophages in asthmatic subjects and its functional involvement in allergen presentation to autologous TH2 lymphocytes. J Allergy Clin Immunol. 1999; 103(6):1136-42.
13. Peters A, Burkett P R, Sobel R A, Buckley C D, Watson S P, Bettelli E, et al. Podoplanin negatively regulates CD4⁺ effector T cell responses. J Clin Invest. 2015; 125(1):129-40.
14. Chihara N, Madi A, Kondo T, Zhang H, Acharya N, Singer M, et al. Induction and transcriptional regulation of the co-inhibitory gene module in T cells. Nature. 2018; 558(7710):454-9.
15. Astarita J L, Acton S E, and Turley S J. Podoplanin: emerging functions in development, the immune system, and cancer. Front Immunol. 2012; 3:283.
16. Suzuki-Inoue K, Fuller G L, Garcia A, Eble J A, Pohlmann S, Inoue O, et al. A novel Syk-dependent mechanism of platelet activation by the C-type lectin receptor CLEC-2. Blood. 2006; 107(2):542-9.
17. Colonna M, Samaridis J, and Angman L. Molecular characterization of two novel C-type lectin-like receptors, one of which is selectively expressed in human dendritic cells. Eur J Immunol. 2000; 30(2):697-704.
18. Heng T S, Painter M W, and Immunological Genome Project C. The Immunological Genome Project: networks of gene expression in immune cells. Nat Immunol. 2008; 9(10):1091-4.
19. Jablonski K A, Amici S A, Webb L M, Ruiz-Rosado Jde D, Popovich P G, Partida-Sanchez S, et al. Novel Markers to Delineate Murine M1 and M2 Macrophages. PLoS One. 2015; 10(12):e0145342.
20. Cueni L N, Chen L, Zhang H, Marino D, Huggenberger R, Alitalo A, et al. Podoplanin-Fc reduces lymphatic vessel formation in vitro and in vivo and causes disseminated intravascular coagulation when transgenically expressed in the skin. Blood. 2010; 116(20):4376-84.
21. Misharin A V, Morales-Nebreda L, Reyfman P A, Cuda C M, Walter J M, McQuattie-Pimentel A C, et al. Monocyte-derived alveolar macrophages drive lung fibrosis and persist in the lung over the life span. J Exp Med 2017; 214(8):2387-404.
22. Mould K J, Barthel L, Mohning M P, Thomas S M, McCubbrey A L, Danhorn T, et al. Cell Origin Dictates Programming of Resident versus Recruited Macrophages during Acute Lung Injury. Am J Respir Cell Mol Biol. 2017; 57(3):294-306.
23. Gardai S J, Xiao Y Q, Dickinson M, Nick J A, Voelker D R, Greene K E, et al. By binding SIRPalpha or calreticulin/CD91, lung collectins act as dual function surveillance molecules to suppress or enhance inflammation. Cell. 2003; 115(1):13-23.
24. Janssen W J, McPhillips K A, Dickinson M G, Linderman D J, Morimoto K, Xiao Y Q, et al. Surfactant proteins A and D suppress alveolar macrophage phagocytosis via interaction with SIRP alpha. Am J Respir Crit Care Med. 2008; 178(2):158-67.
25. Mourao-Sa D, Robinson M J, Zelenay S, Sancho D, Chakravarty P, Larsen R, et al. CLEC-2 signaling via Syk in myeloid cells can regulate inflammatory responses. Eur J Immunol. 2011; 41(10):3040-53.
26. Willart M A, Deswarte K, Pouliot P, Braun H, Beyaert R, Lambrecht B N, et al. Interleukin-1alpha controls allergic sensitization to inhaled house dust mite via the epithelial release of G M-CSF and IL-33. J Exp Med. 2012; 209(8):1505-17.
27. Peters A, Pitcher L A, Sullivan J M, Mitsdoerffer M, Acton S E, Franz B, et al. Th17 cells induce ectopic lymphoid follicles in central nervous system tissue inflammation. Immunity. 2011; 35(6):986-96.
28. Hirota K, Turner J E, Villa M, Duarte J H, Demengeot J, Steinmetz O M, et al. Plasticity of Th17 cells in Peyer's patches is responsible for the induction of T cell-dependent IgA responses. Nat Immunol. 2013; 14(4):372-9.
29. Lax S, Rayes J, Wichaiyo S, Haining E J, Lowe K, Grygielska B, et al. Platelet CLEC-2 protects against lung injury via effects of its ligand podoplanin on inflammatory alveolar macrophages in the mouse. Am J Physiol Lung Cell Mol Physiol. 2017; 313(6):L1016-L29.
30. Rayes J, Lax S, Wichaiyo S, Watson S K, Di Y, Lombard S, et al. The podoplanin-CLEC-2 axis inhibits inflammation in sepsis. Nat Commun. 2017; 8(1):2239.
31. Cui Y, Liu K, Monzon-Medina M E, Padera R F, Wang H, George G, et al. Therapeutic lymphangiogenesis ameliorates established acute lung allograft rejection. J Clin Invest. 2015; 125(11):4255-68.
32. Tsukiji N, Inoue O, Morimoto M, Tatsumi N, Nagatomo H, Ueta K, et al. Platelets play an essential role in murine lung development through Clec-2/podoplanin interaction. Blood. 2018; 132(11):1167-79.
33. Krausgruber T, Blazek K, Smallie T, Alzabin S, Lockstone H, Sahgal N, et al. IRF5 promotes inflammatory macrophage polarization and TH1-TH17 responses. Nat Immunol. 2011; 12(3):231-8.
34. Moore W C, Hastie A T, Li X, Li H, Busse W W, Jarjour N N, et al. Sputum neutrophil counts are associated with more severe asthma phenotypes using cluster analysis. J Allergy Clin Immunol. 2014; 133(6):1557-63 e5.
35. Loza M J, Djukanovic R, Chung K F, Horowitz D, Ma K, Branigan P, et al. Validated and longitudinally stable asthma phenotypes based on cluster analysis of the ADEPT study. Respir Res. 2016; 17(1):165.
36. Draijer C, Boorsma C E, Robbe P, Timens W, Hylkema M N, Ten Hacken N H, et al. Human asthma is characterized by more IRF5+ M1 and CD206+ M2 macrophages and less IL-10+ M2-like macrophages around airways compared with healthy airways. J Allergy Clin Immunol. 2017; 140(1):280-3 e3.
37. Picelli S, Faridani O R, Bjorklund A K, Winberg G, Sagasser S, and Sandberg R. Full-length RNA-seq from single cells using Smart-seq2. Nat Protoc. 2014; 9(1):171-81.
38. Bray N L, Pimentel H, Melsted P, and Pachter L. Near-optimal probabilistic RNA-seq quantification. Nat Biotechnol. 2016; 34(5):525-7.
39. Soneson C, Love M I, and Robinson M D. Differential analyses for RNA-seq: transcript-level estimates improve gene-level inferences. F1000Res. 2015; 4:1521.
40. Love M I, Huber W, and Anders S. Moderated estimation of fold change and dispersion for RNA-seq data with DESeq2. Genome Biol. 2014; 15(12):550.
41. Wallrapp A, Riesenfeld S J, Burkett P R, Abdulnour R E, Nyman J, Dionne D, et al. The neuropeptide NMU amplifies ILC2-driven allergic lung inflammation. Nature. 2017; 549(7672):351-6.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of modulating a Type 2 and/or Type 3 inflammatory response in a subject in need thereof, comprising administering to the subject a therapeutically effective amount of one or more agents capable of modulating CLEC-2 signaling.

2. The method of claim 1, wherein the Type 2 inflammatory response is an IL-33 mediated response.

3. The method of claim 1 or 2, wherein the one or more agents modulate the interaction of CLEC-2 with PDPN.

4. The method of claim 3, wherein the interaction is blocked, whereby a Type 2 and/or Type 3 inflammatory immune response is enhanced.

5. The method of any of claims 1 to 4, wherein the one or more agents is a recombinant PDPN protein or protein fragment.

6. The method of claim 5, wherein the recombinant PDPN fragment comprises the extracellular domain of PDPN.

7. The method of claim 5, wherein the recombinant PDPN is modified to be more stable.

8. The method of claim 7, wherein the recombinant PDPN is a Fc fusion protein.

9. The method of any of claims 1 to 4, wherein the one or more agents is a recombinant CLEC-2 protein or protein fragment.

10. The method of claim 9, wherein the recombinant CLEC-2 fragment comprises the extracellular domain of CLEC-2.

11. The method of claim 9, wherein the recombinant CLEC-2 is a Fc fusion protein.

12. The method of any of claims 1 to 3, wherein the one or more agents is a CLEC-2 signaling agonist, whereby a Type 2 inflammatory immune response is suppressed or homeostasis is maintained.

13. The method of claim 12, wherein the CLEC-2 agonist increases expression or activity of CLEC-2 in macrophages and/or monocytes.

14. The method of claim 13, wherein the agonist is a CLEC-2 expression vector targeted to macrophages and/or monocytes, wherein the agent is under control of a macrophage and/or monocyte specific promoter.

15. The method of claim 12 or 13, wherein the CLEC-2 agonist binds to CLEC-2.

16. The method of any of claims 1, 2, or 12, wherein the one or more agents modulate the expression, activity, and/or function of one or more genes or gene products differentially expressed upon deletion of CLEC-2 in macrophages.

17. The method of claim 16, wherein the one or more genes are selected from Table 2 or the group consisting of Mthfd2, Itga4, L1cam, 9130019O22Rik, Gal, Clec1b, Mgst3, Tgfb2, Serpina3f, Ifi47, Vcam1, Gbp6, Slamf8, Best1, H2-Q9, Ccr5, Rgs12, H2-DMb1, Tgtp2, Gbp4, Pla2g7, Plxnb2, Cd5l, Enpp5, Ifitm3, Pbx1, Pla2g2d, Tgtp1, AA467197, Ptpro, H2-Q4, Irf5, C3, Mt2, Sqle, Ch25h, Il1a, Rilpl2, Igf1, Cd52, Sc4 mol, Smpdl3b, Fbp1, Pilrb2, C1qa, H2-M2, Cdh1, Cyp1a1, Ccl9, Marco, Plxna1, Cxcl16, Trpm2, Ccdc80 and Cd300a.

18. The method of claim 17, wherein the one or more genes are upregulated upon deletion of CLEC-2 and the one or more agents inhibit the one or more upregulated genes.

19. The method of claim 18, wherein the one or more upregulated genes are selected from the group consisting of: Plxnb2, Sqle, H2-Q9, Tgtp1, Cdh1, H2-M2, Ccdc80, C3, Cd300a, Slamf8, Mt2, Serpina3f, Cd52, Rilpl2, Enpp5, Rgs12, Pla2g7, Gbp4, Irf5, Best1, H2-Q4, Ccl9, Ccr5, Igf1, Cxcl16, C1qa, Trpm2, Vcam1, Fbp1, Ch25h, Marco, AA467197, Pla2g2d, Smpdl3b, H2-DMb1, Gbp6, Tgtp2, Ifi47, Il1a, Sc4 mol, Cyp1a1, Pilrb2, Plxna1, Ifitm3, Pbx1, Cd5l, Ptpro, Ctla4, Ptgs1 and Mmab.

20. The method of any of claims 16 to 19, wherein the one or more agents target alveolar macrophages and/or monocytes.

21. The method of claim 20, wherein the one or more agents are targeted by a macrophage and/or monocyte specific expression vector, wherein the agent is under control of a macrophage and/or monocyte specific promoter.

22. The method of any of claims 1-4, 12, 13 or 15-21, wherein the one or more agents comprise an antibody, small molecule, small molecule degrader, genetic modifying agent, antibody-like protein scaffold, aptamer, protein, or any combination thereof.

23. The method of claim 22, wherein the antibody is a CLEC-2 or PDPN antibody.

24. The method of claim 22, wherein the genetic modifying agent comprises a CRISPR system, RNAi system, a zinc finger nuclease system, a TALE, or a meganuclease.

25. The method of claim 24, wherein the CRISPR system is a Class 1 or Class 2 CRISPR system.

26. The method of claim 25, wherein the Class 2 system comprises a Type II Cas polypeptide.

27. The method of claim 26, wherein the Type II Cas is a Cas9.

28. The method of claim 25, wherein the Class 2 system comprises a Type V Cas polypeptide.

29. The method of claim 28, wherein the Type V Cas is Cas12a, Cas12b, Cas12c, Cas12d (CasY), Cas12e(CasX), or Cas14.

30. The method of claim 25, wherein the Class 2 system comprises a Type VI Cas polypeptide.

31. The method of claim 30, wherein the Type VI Cas is Cas13a, Cas13b, Cas13c or Cas13d.

32. The method of any of claims 25 to 31, wherein the CRISPR system comprises a dCas fused or otherwise linked to a nucleotide deaminase.

33. The method of claim 32, wherein the nucleotide deaminase is a cytidine deaminase or an adenosine deaminase.

34. The method of claim 25, wherein the CRISPR system is a prime editing system.

35. The method of any of claims 1 to 34, wherein the treatment is administered to a mucosal surface.

36. The method of claim 35, wherein the treatment is administered to the lung, nasal passage, trachea, gut, intestine, or esophagus.

37. The method of any of claims 1 to 36, wherein the treatment is administered by aerosol inhalation.

38. The method of any of claims 1 to 36, wherein the treatment is administered by a time-release composition.

39. The method of any of claims 1 to 38, wherein the subject is suffering from or at risk for an allergic inflammatory disease.

40. The method of claim 39, wherein the allergic inflammatory disease is selected from the group consisting of asthma, allergy, allergic rhinitis, allergic airway inflammation, atopic dermatitis (AD), chronic obstructive pulmonary disease (COPD), inflammatory bowel disease (IBD), multiple sclerosis, arthritis, psoriasis, eosinophilic esophagitis, eosinophilic pneumonia, eosinophilic psoriasis, hypereosinophilic syndrome, graft-versus-host disease, uveitis, cardiovascular disease, pain, multiple sclerosis, lupus, vasculitis, chronic idiopathic urticaria and Eosinophilic Granulomatosis with Polyangiitis (Churg-Strauss Syndrome).

41. The method of claim 40, wherein the asthma is selected from the group consisting of allergic asthma, non-allergic asthma, severe refractory asthma, asthma exacerbations, viral-induced asthma or viral-induced asthma exacerbations, steroid resistant asthma, steroid sensitive asthma, eosinophilic asthma and non-eosinophilic asthma.

42. The method of claim 40, wherein the allergy is to an allergen selected from the group consisting of food, pollen, mold, dust mites, animals, and animal dander.

43. The method of claim 40, wherein IBD comprises a disease selected from the group consisting of ulcerative colitis (UC), Crohn's Disease, collagenous colitis, lymphocytic colitis, ischemic colitis, diversion colitis, Behcet's syndrome, infective colitis, indeterminate colitis, and other disorders characterized by inflammation of the mucosal layer of the large intestine or colon.

44. The method of claim 40, wherein the arthritis is selected from the group consisting of osteoarthritis, rheumatoid arthritis and psoriatic arthritis.

45. A method of screening for agents capable of shifting alveolar macrophages to a homeostatic or inflammatory macrophage, comprising contacting macrophages with one or more agents and detecting expression of one or more genes selected from Table 2.

46. A method of detecting a Type 2 and/or type 3 inflammatory state in a subject, comprising detecting in immune cells obtained from the subject the expression or activity of one or more genes selected from Table 2; or the group consisting of Plxnb2, Sqle, H2-Q9, Tgtp1, Cdh1, H2-M2, Ccdc80, C3, Cd300a, Slamf8, Mt2, Serpina3f, Cd52, Rilpl2, Enpp5, Rgs12, Pla2g7, Gbp4, Irf5, Best1, H2-Q4, Ccl9, Ccr5, Igf1, Cxcl16, C1qa, Trpm2, Vcam1, Fbp1, Ch25h, Marco, AA467197, Pla2g2d, Smpdl3b, H2-DMb1, Gbp6, Tgtp2, Ifi47, Il1a, Sc4 mol, Cyp1a1, Pilrb2, Plxna1, Ifitm3, Pbx1, Cd5l, Ptpro, Ctla4, Ptgs1 and Mmab; or the group consisting of Mthfd2, Itga4, L1cam, 9130019O22Rik, Gal, Clec1b, Mgst3, Tgfb2, Serpina3f, Ifi47, Vcam1, Gbp6, Slamf8, Best1, H2-Q9, Ccr5, Rgs12, H2-DMb1, Tgtp2, Gbp4, Pla2g7, Plxnb2, Cd5l, Enpp5, Ifitm3, Pbx1, Pla2g2d, Tgtp1, AA467197, Ptpro, H2-Q4, Irf5, C3, Mt2, Sqle, Ch25h, Il1a, Rilpl2, Igf1, Cd52, Sc4 mol, Smpdl3b, Fbp1, Pilrb2, C1qa, H2-M2, Cdh1, Cyp1a1, Ccl9, Marco, Plxna1, Cxcl16, Trpm2, Ccdc80 and Cd300a, wherein upregulation of upregulated genes in Table 2 and downregulation of downregulated genes in Table 2 indicate an inflammatory state.

47. The method of claim 46, wherein the immune cell is a macrophage.

48. A method of treatment comprising detecting a Type 2 and/or type 3 inflammatory state comprising detecting in immune cells from a subject to be treated the one or more genes of claim 46; and administering to the subject the treatment of any one of claims 1-44 and/or a standard anti-inflammatory treatment if the one or more genes are detected.

49. The method of claim 48, wherein the immune cell is a macrophage.


SEQUENCE LISTING (19 sequences)

SEQ ID NO: 1 - protein, 7 aa, Simian virus 40
PKKKRKV

SEQ ID NO: 2 - protein, 10 aa, Simian virus 40
PKKKRKVEAS

SEQ ID NO: 3 - protein, 16 aa, Artificial sequence
  Synthetic
KRPAATKKAGQAKKKK

SEQ ID NO: 4 - protein, 9 aa, Homo sapiens
PAAKRVKLD

SEQ ID NO: 5 - protein, 11 aa, Homo sapiens
RQRRNELKRSP

SEQ ID NO: 6 - protein, 38 aa, Artificial sequence
  Synthetic
NQSSNFGPMKGGNFGGRSSGPYGGGGQYFAKPRNQGGY

SEQ ID NO: 7 - protein, 42 aa, Artificial sequence
  Synthetic
RMRIZFKNKGKDTAELRRRRVEVSVELRKAKKDEQILKRRNV

SEQ ID NO: 8 - protein, 8 aa, Artificial sequence
  Synthetic
VSRKRPRP

SEQ ID NO: 9 - protein, 8 aa, Artificial sequence
  Synthetic
PPKKARED

SEQ ID NO: 10 - protein, 8 aa, Homo sapiens
PQPKKKPL

SEQ ID NO: 11 - protein, 12 aa, Mus musculus
SALIKKKKKMAP

SEQ ID NO: 12 - protein, 5 aa, Influenza virus NS1
DRLRR

SEQ ID NO: 13 - protein, 7 aa, Influenza virus NS1
PKQKKRK

SEQ ID NO: 14 - protein, 10 aa, Hepatitis virus
RKLKKKIKKL

SEQ ID NO: 15 - protein, 10 aa, Mus musculus
REKKKFLKRR

SEQ ID NO: 16 - protein, 20 aa, Homo sapiens
KRKGDEVDGVDEVAKKKSKK

SEQ ID NO: 17 - protein, 17 aa, Homo sapiens
RKCLQAGMNLEARKTKK

SEQ ID NO: 18 - protein, 288 aa, Artificial sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 19 - protein, 183 aa, Artificial sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS